(19) United States
(12) Patent Application Publication
(10) Pub. No.: US 2022/0315934 A1
(43) Pub. Date: Oct. 6, 2022

(54) HETEROLOGOUS RIBOSOME GENERATION, ASSESSMENT AND COMPOSITIONS THEREOF

(71) Applicant: THE BROAD INSTITUTE, INC., Cambridge, MA (US)

(72) Inventors: Ahmed H. Badran, Cambridge, MA (US); Natalie Kolber, Cambridge, MA (US)

(73) Assignee: THE BROAD INSTITUTE, INC., Cambridge, MA (US)

(21) Appl. No.: 17/626,420

(22) PCT Filed: Jul. 14, 2020
(86) PCT No.: PCT/US2020/041905

§ 371 Date: Jan. 11, 2022

Related U.S. Application Data
(60) Provisional application No. 62/873,957, filed on Jul. 14, 2019.
(60) Provisional application No. 62/924,472, filed on Oct. 22, 2019.

(51) Int. Cl. C12N 15/67; C12N 15/10

(52) U.S. Cl. CPC C12N 15/67; C12N 15/1034

(57) ABSTRACT

The present disclosure relates to compositions and methods that enable enhanced monitoring and improvement of heterologous ribosome activity within a host cell. Specifically, the instant disclosure provides a reporter system that allows for improved monitoring of heterologous ribosome activity in a host cell, via engineering of both heterologous rRNA operon sequences and reporter operon sequences. New transgenic organisms harboring heterologous ribosome operons are also provided, as are methods for identifying agents capable of targeting heterologous ribosomes (e.g., ribosomes of pathogenic organisms, optionally selectively as compared to ribosomes of, e.g., commensal organisms) within a host cell.

### CROSS REFERENCE TO RELATED APPLICATIONS

This application claims the benefit of U.S. Provisional Application No. 62/873,957, filed Jul. 14, 2019, entitled “Heterologous Ribosome Generation, Assessment and Compositions Thereof,” and to U.S. Provisional Application No. 62/924,472, filed Oct. 22, 2019, also entitled “Heterologous Ribosome Generation, Assessment and Compositions Thereof.” The entire contents of the aforementioned applications are incorporated herein by reference.

### STATEMENT REGARDING FEDERALLY SPONSORED RESEARCH

This invention was made with government support under Grant No. OD024590 awarded by the National Institutes of Health and under Grant No. NNH17ZDA001N-EXO awarded by the National Aeronautics and Space Administration. The government has certain rights in the invention.

### FIELD OF THE INVENTION

The invention relates generally to compositions, methods and kits for generating, assessing, improving the activity of and/or identifying compounds that target ribosomes.

### SEQUENCE LISTING

The instant application contains a Sequence Listing which has been filed electronically in ASCII format and is hereby incorporated by reference in its entirety. Said ASCII copy, created on Jul. 14, 2020, is named 52199_542001WO_SeqLis.txt and is 3.41 MB in size. A tabulated description of sequences presented in the instant Sequence Listing is provided in FIG. 17 herein.

### BACKGROUND OF THE INVENTION

Escherichia coli (E. coli) is one of the world's best-characterized organisms. Among many advantages of working with E. coli, it can divide every 20 min in the laboratory under aerobic, nutrient-rich conditions. Pathogenic microbes tend to divide much more slowly—e.g., Syntrophobacter fumaroxidans only doubles in the laboratory every 140 hours (Harmsen et al. Int. J. Syst. Bacteriol. 48: 1383-1388). Working with pathogenic microbes also incurs significant containment costs and carries inherent safety risks.

The extraordinary catalytic capabilities of the ribosome represent a promising avenue for both synthetic biology and for identification of new, ribosome-targeting agents/therapeutics, yet the complexity and essentiality of the ribosome have hindered significant engineering efforts. Despite these limitations, the existence of extensive sequence identity among ribosomal RNAs (rRNAs) from closely related species has enabled limited heterologous rRNA evaluation in engineered E. coli strains via complementation of a genomic ribosome deficiency. However, unsuccessful rRNA complementation has thus far failed to guide the optimization of refractory ribosomes. A need exists for methods and compositions capable of enhancing generation of host cells that harbor heterologous ribosomes and for improving ribosomal activity (and assessment of such activity) in such host cells, for synthetic biology/evolution and ribosome-targeting antibiotic screening purposes, among others.

### SUMMARY OF THE INVENTION

The current disclosure relates, at least in part, to discovery and generation of an improved system that allows for assessment and evolution/improvement of heterologous ribosome activity within a host cell (optionally, a highly tractable host cell). Specifically, the instant disclosure provides a reporter system that enables monitoring of heterologous ribosome activity in a host cell, via engineering of heterologous rRNA operon sequences and reporter operon sequences. The instant disclosure has further identified that activity of a heterologous rRNA operon can be improved in a host cell by replacing intergenic sequences of the heterologous operon with corresponding host cell intergenic sequences, and that heterologous ribosome activity can be enhanced via introduction of discrete panels of heterologous r-proteins. The instant disclosure has further provided numerous heterologous rRNA-harboring genetic organisms, which enable improved screening approaches for ribosome-targeting agents and also allow for improved synthetic ribosome evolution approaches to be performed upon such cells. The instant disclosure therefore also provides screening approaches for identification of ribosome-targeting antibiotic agents, including, e.g., identification of narrow-spectrum antibiotic agents that preferentially target ribosomes of pathogenic microbes, as compared to commensal microorganisms.

In one aspect, the instant disclosure provides a method for increasing the activity and/or improving the maturation of a non-host cell ribosomal RNA (rRNA) in a host cell, where the non-host cell rRNA is encoded by a nucleic acid sequence harboring both rRNA coding sequences and intergenic sequences, the method involving replacing the intergenic sequences of the nucleic acid sequence harboring both rRNA coding sequences and intergenic sequences with intergenic sequences of the host cell, thereby increasing the activity and/or improving the maturation of the non-host cell rRNA in the host cell.

In one embodiment, the host cell is Escherichia coli. Optionally, the E. coli strain has a genomic deletion for rRNA sequences. Optionally, the E. coli strain carries a counter-selectable plasmid harboring E. coli rRNA sequences. Optionally, the E. coli strain is SQ171.

In certain embodiments, the non-host cell is Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica or Shigella spp. (e.g., Shigella flexneri, Shigella dysenteriae, Shigella sonnei, Shigella boydii).

In certain embodiments, the non-host cell is a commensal microbe. Optionally, the commensal microbe is of the phylum Firmicutes, Bacteroidetes, Bifidobacteria, Eubacteria, Ruminococcus, Lactobacillus, Peptococcus, Proteobacteria, Verrumicrobia, Actinobacteria, Fusobacteria or Cyanobacteria, or a combination of phyla thereof.

In another embodiment, the host cell is Bacillus subtilis. Optionally, the B. subtilis strain has a genomic deletion for rRNA sequences. Optionally, the B. subtilis strain carries a counter-selectable plasmid harboring B. subtilis rRNA sequences.

In some embodiments, the non-host cell is Mycobacterium tuberculosis, Bifidobacterium longum, Veillonella parvula, Clostridium difficile, Escherichia coli, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica or Shigella spp. (e.g., Shigella flexneri, Shigella dysenteriae, Shigella sonnei, Shigella boydii).

In certain embodiments, the non-host cell is Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris or Haemophilus influenza.

In one embodiment, the nucleic acid sequence having both rRNA coding sequences and intergenic sequences includes non-host cell 165, 23S and 5S rRNA sequences. Optionally, the non-host cell 165, 23S and 5S rRNA sequences are under the control of an inducible promoter. Optionally, the inducible promoter is an aTc-inducible promoter or an IPTG-inducible promoter.

In a related embodiment, the host cell includes a nucleic acid sequence harboring an orthogonal-ribosome binding site (o-RBS) positioned upstream of a reporter sequence. Optionally, the reporter sequence encodes a fluorescent protein. Optionally, the fluorescent protein is a Sirius fluorescent protein, mTagBFP2, a blue fluorescent protein (BFP), Sapphire fluorescent protein, mCerulean, a yellow fluorescent protein (YFP), LSS-mKate2, MiCy, green a fluorescent protein (GFP), mEmerald, Venus, mPapaya, mScarlet-1, mCherry, mRFP, Katushka-9-5, mCarmine, mMaroon1, or E2-Crimson. Optionally, the reporter sequence encodes a chemiluminescent protein. Optionally, the chemiluminescent protein is a luciferase protein. Optionally, the nucleic acid sequence having an o-RBS positioned upstream of a reporter sequence is under the control of an inducible promoter. Optionally, the inducible promoter is an aTc-inducible promoter or an IPTG-inducible promoter. Optionally, the o-RBS reporter sequence is under the control of a PLTet0-1 or a PtetA promoter.

Optionally, the nucleic acid sequence including both rRNA coding sequences and intergenic sequences harbors a non-host cell 16S rRNA sequence that further includes an o-antiRBS sequence. (An exemplary o-antiRBS sequence is 5′-ACCACA-3′ (SEQ ID NO: 406), while a specific example of o-antiRBS-containing sequence, shown in FIG. 7A, is 5′-ATTTTTTCCAACCACAGATCT-3′ (SEQ ID NO. 407).)

In certain embodiments, non-host cell rRNA activity is increased to 50% or more of the level of an appropriate host cell rRNA activity control.

In some embodiments, growth of the host cell is improved.

An additional aspect of the instant disclosure provides a nucleic acid sequence having an aTC-inducible promoter and 165, 23S and 5S rRNA coding sequences, where the 16S sequence further harbors an o-antiRBS sequence.

Another aspect of the instant disclosure provides a rRNA reporter system that includes (a) a first nucleic acid sequence harboring an aTC-inducible promoter and 165, 23S and 5S rRNA coding sequences, where the 16S sequence further includes an o-antiRBS sequence; and (b) a second nucleic acid sequence including an o-RBS sequence and a reporter sequence.

In one embodiment, the second nucleic acid sequence includes an inducible promoter. Optionally, the inducible promoter is an IPTG-inducible promoter.

In certain embodiments, the reporter sequence encodes a green fluorescent protein (GFP), a blue fluorescent protein (BFP), a yellow fluorescent protein (YFP), luciferase or a mRFP (e.g., mCherry).

In one embodiment, the aTC-inducible promoter is a PLtetO-1 or a PtetA promoter.

In another embodiment, the rRNA reporter system further includes a third nucleic acid sequence encoding for S20, S16, S1 and/or S15 r-protein(s).

In certain embodiments, the 165, 23S and 5S rRNA coding sequences are non-E. coli sequences. Optionally, the first nucleic acid sequence further includes intergenic sequences. Optionally, the intergenic sequences are E. coli intergenic sequences. Optionally, the rRNA reporter system further includes a third nucleic acid sequence encoding for non-E. coli S20, S16, S1 and/or S15 r-protein(s) of the same organism as the non-E. coli 16S, 23S and 5S rRNA coding sequences.

Another aspect of the instant disclosure provides a host cell harboring a nucleic acid sequence having non-host cell 16S, 23S and 5S rRNA coding sequences, where the non-host cell is Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii or Shigella spp. (e.g., Shigella flexneri, Shigella dysenteriae, Shigella sonnei, Shigella boydii).

A further aspect of the instant disclosure provides a host cell harboring a nucleic acid sequence that includes non-host cell 165, 23S and 5S rRNA coding sequences, where the non-host cell is a commensal microbe. Optionally, the commensal microbe is of one or more of the following phyla: Firmicutes, Bacteroidetes, Bifidobacteria, Eubacteria, Ruminococcus, Lactobacillus, Peptococcus, Proteobacteria, Verrumicrobia, Actinobacteria, Fusobacteria, and Cyanobacteria.

In one embodiment, the nucleic acid sequence having non-host cell 165, 23S and 5S rRNA coding sequences further includes intergenic sequences. Optionally, the intergenic sequences are host cell intergenic sequences.

In another embodiment, the non-host cell 16S rRNA sequence further includes an o-antiRBS sequence.

In an additional embodiment, the host cell further includes a nucleic acid sequence encoding for S20, S16, S1 and/or S15 r-protein(s) of the non-host cell.

In some embodiments, the host cell further includes a nucleic acid sequence harboring an orthogonal-ribosome binding site (o-RBS) positioned upstream of a reporter sequence. Optionally, the nucleic acid sequence harboring an o-RBS positioned upstream of a reporter sequence is under the control of an inducible promoter.

An additional aspect of the instant disclosure provides a method for increasing the activity of a non-host cell ribosomal RNA (rRNA) in a host cell, the method involving introducing a nucleic acid sequence encoding for S20 and/or S16 r-protein(s) of the non-host cell into the host cell, thereby increasing the activity of the non-host cell rRNA in the host cell.

In one embodiment, the method further involves introducing a nucleic acid sequence encoding for S1 and/or S15 r-protein(s) of the non-host cell into the host cell.

In certain embodiments, the host cell is Escherichia coli. Optionally, the non-host cell is Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica, Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris, Haemophilus influenza or Shigella spp. (e.g., Shigella flexneri, Shigella dysenteriae, Shigella sonnei, Shigella boydii).

In some embodiments, the host cell is Bacillus subtilis. Optionally, the non-host cell is Mycobacterium tuberculosis, Bifidobacterium longum, Veillonella parvula, Clostridium difficile, Escherichia coli, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica, Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris, Haemophilus influenza or Shigella spp. (e.g., Shigella flexneri, Shigella dysenteriae, Shigella sonnei, Shigella boydii).

In one embodiment, the non-host cell is A. baumannii and the nucleic acid sequence encodes for AbS20 and/or AbS16 r-protein(s).

In another embodiment, the non-host cell is A. macleodii and the nucleic acid sequence encodes for AmS20 and AmS16 r-proteins. Optionally, the nucleic acid sequence further encodes for AmS1 and/or AmS15 r-protein(s).

In an additional embodiment, the non-host cell is V. cholerae or M. minitulum and the nucleic acid sequence encodes for S20, S16, S1 and S15 r-proteins of the non-host cell.

In certain embodiments, the non-host cell is P. aeruginosa and the nucleic acid sequence encodes for PaS16 and PaS20 r-proteins. Optionally, the nucleic acid sequence further encodes for PaS1 and/or PaS15 r-protein(s).

In another embodiment, the non-host cell is A. faecalis, B. cenocepacia, N. gonnorrheae, M. ferrooxydans, or C. crescentus and the nucleic acid sequence encodes for cognate non-host cell S16 and S20 r-proteins.

In some embodiments, the nucleic acid sequence encoding for S20 and/or S16 r-proteins of the non-host cell is under the control of a copy-up variant. Optionally, the copy-up variant is RepA E93K or E93R.

In another embodiment, the host cell further includes an o-RBS reporter construct. Optionally, the reporter of the o-RBS reporter construct is under control of an IPTG-inducible promoter.

In one embodiment, a nucleic acid sequence harboring non-host cell 16S, 23S and 5S rRNA sequences expresses the non-host cell rRNA in the host cell. Optionally, the non-host cell 16S, 23S and 5S rRNA sequences are under the control of an inducible promoter. Optionally, the inducible promoter is an aTc-inducible promoter or an IPTG-inducible promoter. Optionally, the host cell includes a nucleic acid sequence harboring an orthogonal-ribosome binding site (o-RBS) positioned upstream of a reporter sequence. Optionally, the nucleic acid sequence harboring an o-RBS positioned upstream of a reporter sequence is under the control of an inducible promoter. Optionally, the inducible promoter is an aTc-inducible promoter or an IPTG-inducible promoter.

In another embodiment, the nucleic acid sequence having non-host cell 16S, 23S and 5S rRNA sequences includes a non-host cell 16S rRNA sequence further including an o-antiRBS sequence.

In certain embodiments, non-host cell rRNA activity is increased to 50% or more of the level of an appropriate host cell rRNA control.

In some embodiments, growth of the host cell is improved.

In a further aspect, the instant disclosure provides a method for identifying a compound capable of modulating the rRNA activity of a pathogenic microbe in a host cell, where the host cell includes (i) a rRNA reporter system having a first nucleic acid sequence harboring 165, 23S and 5S rRNA coding sequences, where the 16S sequence further includes an o-antiRBS sequence; and (ii) a second nucleic acid sequence having an o-RBS sequence and a reporter sequence, the method involving: (a) contacting the host cell with a test compound; and (b) measuring modulation of the reporter sequence in the presence of the test compound, as compared to an appropriate control, thereby identifying the test compound as a compound capable of modulating the rRNA activity of a pathogenic microbe in the host cell.

In certain embodiments, the appropriate control is the rRNA activity of a commensal microbe. Optionally, the rRNAs of pathogenic and commensal microbes are multiplexed in the host cell.

In one embodiment, the test compound reduces pathogenic microbe rRNA activity.

In another embodiment, the test compound, when administered to the pathogenic microbe, reduces growth of the pathogenic microbe.

In certain embodiments, the test compound is a small molecule.

In some embodiments, the host cell further includes a nucleic acid sequence encoding for S20, S16, S1 and/or S15 r-protein(s) of the pathogenic microbe.

In another embodiment, the first nucleic acid sequence harboring 165, 23S and 5S rRNA coding sequences further includes intergenic sequences. Optionally, the intergenic sequences are host cell intergenic sequences.

In one embodiment, the test compound selectively modulates the rRNA activity of the pathogenic microbe in the host cell, as compared to modulation of rRNA activity of a commensal microbe in the host cell.

In some embodiments, a test compound which preferentially inhibits the rRNA activity of a pathogenic microbe as compared to the rRNA activity of a commensal microbe is selected for administration to a subject having or at risk of having the pathogenic microbe.

In another aspect, the instant disclosure provides a method for identifying a compound that does not modulate or only weakly modulates (as compared to a pathogenic microbe) the rRNA activity of a commensal microbe in a host cell having (i) a rRNA reporter system harboring a first nucleic acid sequence including 16S, 23S and 5S rRNA coding sequences, where the 16S sequence further includes an o-antiRBS sequence and (ii) a second nucleic acid sequence harboring an o-RBS sequence and a reporter sequence, the method involving: (a) contacting the host cell with a test compound; and (b) measuring modulation of the reporter sequence in the presence of the test compound, as compared to an appropriate control, thereby identifying the test compound as a compound that does not modulate or only weakly modulates (as compared to a pathogenic microbe) the rRNA activity of the commensal microbe in the host cell.

Another aspect of the instant disclosure provides an E. coli cell harboring mutated forms of 23S rRNA genes rrlA, rrlB, rrlC, rrlD, rrlE, rrlG and rrlH.

In embodiments, the E. coli cell further comprises a superfolder GFP (sfGFP) reporter.

In certain embodiments, at least one 23S rRNA gene of E. coli cell genes rrlA, rrlB, rrlC, rrlD, rrlE, rrlG and rrlH harbors an A2058U mutation.

In embodiments, the E. coli cell is erythromycin-resistant.

In some embodiments, the E. coli cell further includes an orthogonal large subunit ribosome and/or an orthogonal small subunit ribosome.

Another aspect of the instant disclosure provides a method for identifying the presence and/or extent of association between an orthogonal SSU and a host cell LSU, the method involving: contacting a host cell of the disclosure having a host cell LSU and harboring a nucleic acid sequence that encodes for an orthogonal SSU capable of being expressed in the host cell, contacting the host cell harboring the orthogonal SSU with erythromycin; and observing the erythromycin sensitivity of the host cell harboring the orthogonal SSU, where: (a) erythromycin sensitivity of the host cell harboring the orthogonal SSU indicates high levels of exchange between the orthogonal SSU and the host cell LSU; and (b) erythromycin resistance of the host cell harboring the orthogonal SSU indicates low levels of exchange between the orthogonal SSU and the host cell LSU (i.e., the orthogonal SSU preferentially associates with the host cell LSU), thereby identifying association between the orthogonal SSU and the host cell LSU.

In one embodiment, the host cell is an E. coli cell.

An additional aspect of the instant disclosure provides a method for enhancing translation in a host cell of an orthogonal nucleic acid harboring a reporter sequence, where the reporter sequence has a 5′ end and a 3′ end, the method involving attaching a sfGFP sequence at the 5′ end of the reporter sequence, thereby enhancing translation of the orthogonal nucleic acid sequence in the host cell.

In one embodiment, the sfGFP sequence includes or is a sequence encoding for SEQ ID NO: 409 (N-MSKGEELFTG-C). Optionally, the sfGFP sequence includes or is SEQ ID NO: 408 (5′-ATGAGCAAAGGTGAAGAACTGTTTACCGGC-3′).

A further aspect of the instant disclosure provides a nucleic acid sequence that includes a first sequence having an o-antiRBS sequence, the first sequence being operably linked to a second sequence that includes a sfGFP sequence having a 5′ and a 3′ end, where the 3′ end of the sfGFP sequence is attached to the 5′ end of a reporter nucleic acid sequence having a 5′ and a 3′ end.

In one embodiment, the reporter nucleic acid sequence encodes a fluorescent protein. Optionally, the fluorescent protein is a Sirius fluorescent protein, mTagBFP2, a blue fluorescent protein (BFP), a Sapphire fluorescent protein, mCerulean, a yellow fluorescent protein (YFP), LSS-mKate2, MiCy, a green fluorescent protein (GFP) (optionally, a superfolder green fluorescent protein (sfGFP)), mEmerald, Venus, mPapaya, mScarlet-1, mCherry, mRFP, Katushka-9-5, mCarmine, mMaroon1, or E2-Crimson.

In another embodiment, the reporter nucleic acid sequence encodes a chemiluminescent protein. Optionally, the chemiluminescent protein is a luciferase protein.

### Definitions

The term “host cell” is used herein to denote any cell, wherein any foreign or exogenous genetic material has been introduced. In its broadest sense, “host cell” is used to denote a cell which has been genetically manipulated. In certain embodiments, “host cell” refers to a microbe, optionally a prokaryotic cell, optionally a tractable prokaryotic cell (e.g., E. coli, B. subtilis, etc.).

As used herein, “heterologous sequence” or “heterologous protein” (e.g., heterologous ribosome) means any sequence or protein other than the one that naturally occurs within a host cell (optionally, in a host cell that has not been genetically modified). In certain embodiments, a heterologous sequence or protein is one for which a corresponding homologous sequence or protein exists within an unmodified host cell.

As used herein, the term “pathogenic microbe” refers to a biological microorganism that is capable of producing an undesirable effect upon a host animal, and includes, for example, without limitation, bacteria, viruses, bacterial spores, molds, mildews, fungi, and the like. This includes all such biological microorganisms, regardless of their origin or of their method of production, and regardless of whether they exist in facilities, in munitions, weapons, or elsewhere. In certain embodiments, the pathogenic microbe of the instant disclosure is a pathogenic bacteria.

As used herein, the term “commensal microbe” refers to a biological microorganism that lives on or in another organism without causing harm. A commensal microbe may refer, without limitation, to bacteria, viruses, fungi, and the like. The term therefore includes all such biological microorganisms, regardless of their origin or of their method of production, and regardless of where they exist. In certain embodiments, the commensal microbe of the instant disclosure is a commensal bacteria.

As used herein, the term “reporter gene” or “reporter nucleic acid” sequence (including “reporter sequence” where reference to a nucleic acid sequence is clear) refers to genes or nucleic acid sequences that enable the detection or measurement of gene expression. Reporter genes and/or reporter nucleic acid sequences may be recombined with regulatory sequences and/or genes of interest, e.g., to report expression, location and/or levels. In some embodiments of the present disclosure, the reporter nucleic acid sequence(s) is a gene encoding a fluorescent or chemiluminescent protein. In some embodiments, a “tag” sequence of the superfolder GFP nucleic acid sequence (“sfGFP” tag nucleic acid sequence is 5′-ATGAGCAAAGGTGAAGAACTGTTTACCGGC-3′ (SEQ ID NO: 408), which encodes for amino acid sequence N-MSKGEELFTG-C(SEQ ID NO: 409)) is employed, as the sfGFP tag sequence was surprisingly identified to enhance the translation of other associated proteins in a host cell.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

By “homologous sequence” is meant a nucleotide sequence that is shared by one or more polynucleotide sequences, such as genes, gene transcripts and/or non-coding polynucleotides. For example, a homologous sequence can be a nucleotide sequence that is shared by two or more genes encoding related but different proteins, such as different members of a gene family, different protein epitopes, different protein isoforms or completely divergent genes, such as a cytokine and its corresponding receptors. A homologous sequence can be a nucleotide sequence that is shared by two or more non-coding polynucleotides, such as noncoding DNA or RNA, regulatory sequences, introns, and sites of transcriptional control or regulation. Homologous sequences can also include conserved sequence regions shared by more than one polynucleotide sequence. Homology does not need to be perfect homology (e.g., 100%), as partially homologous sequences are also contemplated by the instant disclosure (e.g., 99%, 98%, 97%, 96%, 95%, 94%, 93%, 92%, 91%, 90%, 89%, 88%, 87%, 86%, 85%, 84%, 83%, 82%, 81%, 80% etc.). Indeed, design and use of the nucleotide sequences of the instant disclosure contemplates the possibility of using nucleotide sequences that are, e.g., only 99%, 98%, 97%, 96%, 95%, 94%, 93%, 92%, 91%, 90%, 89%, 88%, 87%, 86%, 85%, 84%, 83%, 82%, 81%, 80% etc. homologous to nucleotide sequences recited herein. Indeed, it is contemplated that nucleotide sequences with insertions, deletions, and single point mutations relative to the specific sequences disclosed herein can also be effective, e.g., for use in nucleic acid constructs (and in certain embodiments, in encoded polypeptide sequences) of the instant disclosure. In addition, it is expressly contemplated that nucleotide and/or amino acid sequences with analog substitutions or insertions can also be employed.

Sequence identity may be determined by sequence comparison and alignment algorithms known in the art. To determine the percent identity of two nucleic acid sequences (or of two amino acid sequences), the sequences are aligned for comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, a gapped alignment, the alignment is optimized by introducing appropriate gaps, and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, a global alignment the alignment is optimized by introducing appropriate gaps, and percent identity is determined over the entire length of the sequences aligned. (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1C show the assessment of heterologous rRNA activity via SQ171 complementation. FIG. 1A shows a schematic representation of the SQ171 complementation assay. SQ171 E. coli cells lack all 7 genomic rRNA operons and maintain a single rrnC operon on a SacB counter-selectable plasmid. Introduction of a heterologous rRNA (rrnX) and depletion of the E. coli rrnC plasmid using sucrose yielded cells that relied upon the heterologous ribosome for survival. FIG. 1B shows the growth time course of SQ171 cells bearing increasingly divergent heterologous rRNAs (n=2-8). FIG. 1C shows the correlation between heterologous 16S rRNA sequence identity to E. coli (%) and SQ171 fitness (doublings per hour) upon complementation (99% CI, R2=0.62) (n=1-8). E. coli rRNA control plotted in gray. Data represent the mean and standard deviation of 1-8 biological replicates. Complete SQ171 complementation data is reported in FIG. 14 below.

FIGS. 2A to 2F show the quantification of heterologous rRNA function using orthogonal translation. FIG. 2A shows a schematic representation of the orthogonal translation circuit. A superfolder GFP (sfGFP) reporter incorporated an o-RBS exclusively recognized by ribosomes that encoded the complementary o-antiRBS, yielding a quantifiable fluorescent readout for heterologous ribosome activity. FIG. 2B shows the comparison of wt-antiRBS and o-antiRBS E. coli ribosomes translating the o-RBS sfGFP reporter (n=5). FIG. 2C shows the inducer dependence of the orthogonal translation circuit (n=2). FIG. 2D shows the heterologous rRNA activities of 34 rRNA operons in addition to the E. coli o-rRNA control as quantified by orthogonal translation (n=8). FIG. 2E shows the correlation between 16S rRNA sequence identity to E. coli (%) and activity in the o-translation genetic circuit (normalized to E. coli o-rRNA) for 15 functional heterologous o-rRNAs (mean activity ≥5%), illustrating a correlation between orthogonal translation activity and 16S identity (99% CI, R2=0.79, n=8). FIG. 2F shows the correlation between activity in the orthogonal translation circuit and fitness in SQ171 strain complementation assays for 21 heterologous rRNAs in addition to E. coli, illustrating a linear relationship (99% CI, R2=0.47, n=1-8). E. coli rRNA controls plotted in gray. Data reflect the mean and standard deviation of 1-8 biological replicates. Comprehensive SQ171 complementation and o-translation data reported in FIG. 14 below.

FIGS. 3A to 3E show the effects of o-rRNA intergenic sequence replacement on heterologous translation. FIG. 3A shows the per-base sequence conservation across 34 evaluated rRNA operons, demonstrating limited conservation in intergenic regions as compared to structural rRNA genes. FIG. 3B shows a schematic representation of the intergenic sequence replacement strategy. FIG. 3C shows the effects of intergenic sequence replacement on o-rRNAs with high 16S rRNA sequence identity to E. coli (96.2-99.6%), as well as A. macleodii (85.9%), illustrating a minimal effect on orthogonal translation (n=8). FIG. 3D shows the effects of intergenic sequence replacement on o-rRNAs with intermediate 16S rRNA sequence identity to E. coli (81.5-97.4%), illustrating a significant effect on intergenic sequence replacement (n=8). FIG. 3E shows the correlation between activity in the o-translation circuit and fitness in SQ171 strain complementation assays for 21 rRNAs evaluated after intergenic sequence replacement, illustrating a linear relationship. E. coli rRNA control plotted in gray (99% CI, R2=0.84, n=3-8). Data reflect the mean and standard deviation of 3-8 biological replicates. Comprehensive SQ171 complementation and o-translation data are reported in FIG. 14 below.

FIGS. 4A to 4F show that cognate r-proteins complementation improved heterologous o-rRNA activity. FIG. 4A shows a schematic representation of natural r-protein genomic organization for a given microbial genome and corresponding plasmid architecture for heterologous o-rRNA complementation. FIG. 4B shows that A. baumannii AO1 enhanced cognate heterologous rRNA activity (n=4-12). FIG. 4C shows that A. baumannii S20 and S16 enhanced A. baumannii o-ribosome activity to levels comparable to E. coli o-ribosomes. FIG. 4D shows that A. macleodii AO1 similarly improved cognate heterologous rRNA activity (n=8-12). AO2 was expressed with L19 deleted due to observed toxicity (see FIGS. 10A and 10B below, n=8-12). FIG. 4E shows that cognate S1 or S15, alongside S20 and S16, maximized A. macleodii o-ribosome activity (n=8-16). FIG. 4F shows cognate S20, S16, S1, and S15 supplementation alongside cognate heterologous o-rRNAs. Toxicity was observed when expressing the four proteins together in A. macleodii and A. baumannii (FIG. 11A, n=8). Data reflect the mean and standard deviation of 4-16 biological replicates. Comprehensive o-translation data is reported in FIG. 14 below.

FIGS. 5A to 5F show that phylogenetically-guided determination of cognate r-proteins improved highly divergent heterologous o-ribosomes in E. coli. FIG. 5A shows that no single contiguous operon significantly improved translation activity of o-rRNAs derived from B. cenocepacia (n=8). FIG. 5B shows that no single contiguous operon significantly improved translation activity of o-rRNAs derived from R. parkeri (n=8). FIG. 5C shows that no single contiguous operon significantly improved translation activity of o-rRNAs derived from E. faecalis (n=4). FIG. 5D shows that only 2 of the identified 5 regions with high sequence divergence between E. coli and E. faecalis 16S rRNAs abrogated E. coli o-translation when replaced with cognate E. faecalis sequences (n=4). FIG. 5E shows that cognate S2, S8, S18, S12, 520, S16, and S17 (which directly contacted h9, h10, and h262) allowed for a significant increase in E. faecalis o-rRNA translation. Further analysis showed that S8 and S18 were not required for this increase in activity (n=4). FIG. 5F shows that r-proteins S2, S12, 520, S16, and S17 collectively improved cognate o-rRNA translation for highly divergent species, but were not as effective as S20 and S16 alone for less divergent species. E. faecalis cognate r-proteins were expressed from a low copy number backbone (WT RepA SC101 origin) to limit toxicity (n=8). Data reflect the mean and standard deviation of 4-8 biological replicates. Comprehensive o-translation data is reported in FIG. 14 below.

FIGS. 6A to 6G show benchmarking and extension of the orthogonal reporter system. FIG. 6A shows that the induction of E. coli o-rRNA did not have a significant effect on host growth rate (for OD, n=5; for sfGFP, n=2). FIG. 6B shows that the o-sfGFP (34) reporter used throughout this study demonstrated robust signal-to-noise upon o-rRNA induction, n=5. FIGS. 6C to 6E show that additional orthogonal reporters demonstrated dynamic ranges comparable to or exceeding that of sfGFP. FIG. 6C shows that Photorhabdus luminescens xluxAB (48) demonstrated a dynamic range exceeding that of sfGFP, n=8. FIG. 6D shows that mTagBFP2 (55) demonstrated a dynamic range comparable to that of sfGFP, n=8. FIG. 6E shows that Venus (56) demonstrated a dynamic range comparable to that of sfGFP, n=8. Conversely, an orthogonal reporter incorporating mCherry (57) showed low signal-to-noise. FIG. 6F shows that replacement of successive codons at the mCherry N-terminus with their sfGFP counterparts yielded a gradual improvement in signal, n=8. FIG. 6G shows that replacement of successive codons at the mCherry N-terminus with their sfGFP counterparts yielded a gradual improvement in dynamic range, n=8. FIG. 6H shows a refactored mCherry orthogonal reporter in which the first 10 codons were replaced with the cognate sfGFP and in which the signal had significantly improved dynamic range, n=8. Data reflect the mean and standard deviation of 2-8 biological replicates.

FIGS. 7A to 7P show that a sfGFP-derived leader sequence improved the function of orthogonal reporters. FIG. 7A shows a schematic illustrating the O-antiRBS, 10-aa sfGFP-derived tag, and N-terminus of a fluorescent protein. Sequences shown for the O-antiRBS and sfGFP-derived tags are: o-antiRBS: 5′-ACCACA-3′ (SEQ ID NO: 406), with indicated full flanking sequence, 5′-ATTTTTTCCAACCACAGATCT-3′ (SEQ ID NO: 407); and sfGFP-derived tag: 5′-ATGAGCAAAGGTGAAGAACTGTTTACCGGC-3′ (SEQ ID NO: 408), which encodes for N-MSKGEELFTG-C(SEQ ID NO: 409). When appended to the N-terminus of 15 fluorescent proteins, the tag affected orthogonal translation of the reporter. FIG. 7B shows the translation of Sirius (58) with and without the sfGFP leader in the presence of 0 and 1000 ng/ml aTC. FIG. 7C shows the translation of mTagBFP2 (55) with and without the sfGFP leader in the presence of 0 and 1000 ng/ml aTC. FIG. 7D shows the translation of mCerulean (59) with and without the sfGFP leader in the presence of 0 and 1000 ng/ml aTC. FIG. 7E shows the translation of MiCy (60) with and without the sfGFP leader in the presence of 0 and 1000 ng/ml aTC. FIG. 7F shows the translation of mEmerald (61) with and without the sfGFP leader in the presence of 0 and 1000 ng/ml aTC. FIG. 7G shows the translation of Sapphire (61) with and without the sfGFP leader in the presence of 0 and 1000 ng/ml aTC. FIG. 7H shows the translation of Venus (56) with and without the sfGFP leader in the presence of 0 and 1000 ng/ml aTC. FIG. 7I shows the translation of mPapaya (62) with and without the sfGFP leader in the presence of 0 and 1000 ng/ml aTC. FIG. 7J shows the translation of mScarlet-I (63) with and without the sfGFP leader in the presence of 0 and 1000 ng/ml aTC. FIG. 7K shows the translation of LSS-mKate2 (64) with and without the sfGFP leader in the presence of 0 and 1000 ng/ml aTC. FIG. 7L shows the translation of mCherry (57) with and without the sfGFP leader in the presence of 0 and 1000 ng/ml aTC. FIG. 7M shows the translation of Katusha-9-5 (65) with and without the sfGFP leader in the presence of 0 and 1000 ng/ml aTC. FIG. 7N shows the translation of E2-Crimson (66) with and without the sfGFP leader in the presence of 0 and 1000 ng/ml aTC. FIG. 7O shows the translation of mMaroon1 (67) with and without the sfGFP leader in the presence of 0 and 1000 ng/ml aTC. FIG. 7P shows the translation of mCarmine (68) with and without the sfGFP leader in the presence of 0 and 1000 ng/ml aTC. Generally, addition of the leader tag led to an improvement in absolute signal (average improvement 2.7-fold) and/or dynamic range (average improvement 1.5-fold). Data reflect the mean and standard deviation of 8 biological replicates.

FIG. 8 shows that divergent o-rRNA activities were not improved following intergenic sequence replacement. A comparison of o-sfGFP translation activity before and after intergenic sequence replacement for o-rRNAs derived from increasingly divergent microorganisms (69.8-82.3% 16S rRNA sequence identity to E. coli) is shown, wherein limited improvement was observed following intergenic sequence replacement. Data reflect the mean and standard deviation of 8 biological replicates. Comprehensive o-translation data is reported in FIG. 14 below.

FIGS. 9A to 9E show that protein supplementation significantly improved A. baumannii o-rRNA function. FIG. 9A shows A. baumannii heterologous o-rRNA activity was improved following AO1 induction, yielding comparable activity levels as supplementation with all cognate SSU r-proteins (S1-S21; n=4). FIG. 9B shows A. baumannii heterologous o-rRNA activity improvement further depended upon AO1 copy number, indicating insufficient r-protein production at low copy numbers. Labels indicate RepA genotypes and numbers in parentheses indicate the corresponding copy numbers (41) (n=4). FIG. 9C shows single r-protein deletion from AO1 did not adversely affect A. baumannii heterologous o-rRNA activity, indicating that more than a single r-protein on this plasmid complemented o-rRNA function, n=4. FIG. 9D shows that o-sfGFP production using an E. coli o-rRNA was inversely proportional to mCherry production using E. coli native ribosomes, indicating that r-protein overexpression had pleiotropic effects on o-ribosome activity (99% CI, R2=0.73, n=8). FIG. 9E shows E. coli o-rRNA regulation by P_{LtetO-1} (69) and PtetA. Improved signal and reduced variability was observed with PtetA, the native promoter found in the Tn10 transposon. P_{LtetO-1}-dependent variability was a result of promoter recombination between identical TetR operators (not shown) during cell passaging, n=32. Data reflect the mean and standard deviation of 4-32 biological replicates.

FIGS. 10A to 10E demonstrate the dissection of large subunit (LSU) r-proteins that improved A. macleodii o-rRNA activity. FIG. 10A shows that single r-proteins (from AO2) expressed alongside cognate A. macleodii o-rRNA revealed that L19 was responsible for the observed toxicity from AO2, where removal of L19 (AO2 ΔL19) mitigated a significant fraction of the observed growth reduction. FIG. 10B shows that no single r-protein from AO2 significantly enhanced A. macleodii o-rRNA activity. FIG. 10C shows that single deletions from AO2 did not reveal any variants that differed significantly in effect on o-rRNA activity. FIG. 10D shows that truncation variants from the 5′ end of the artificial operon did not reveal any variants that differed significantly in effect on o-rRNA activity. FIG. 10E shows that truncation variants from the 3′ end of the artificial operon did not reveal any variants that differed significantly in effect on o-rRNA activity. These data collectively suggested that the observed improvement relied on the concerted action of numerous r-proteins from AO2. Data reflect the mean and standard deviation of 8 biological replicates.

FIGS. 11A to 11C show dissection of the contributions of the identified r-proteins 520, S16, S1, and S15. FIG. 11A Expression of cognate S20, S16, S1, and S15 combinations alongside numerous heterologous o-rRNAs. A. macleodii and A. baumannii cognate S20, S16, S1, and S15 limit the growth of the E. coli host when co-expressed, as indicated by culture density after overnight growth, whereas most other r-proteins evaluated are well tolerated. NT=not tested. b) Both cognate r-proteins S20 and S16 are necessary for maximal sfGFP expression using o-rRNAs from more divergent microorganisms: N. gonorrheae (81.8% 16S rRNA sequence identity to E. coli), M. ferrooxydans (80.1%), and C. crescentus (79.3%). However, S20 and S16 are functionally redundant when expressed alongside more related o-rRNAs to E. coli. c) The combination of S20, S16, S1, and S15 is necessary for maximal activity using V. cholerae (90.3% 16S identity to E. coli) and M. minutulum (85.3%) o-rRNAs. For more phylogenetically distant o-rRNAs, no additional improvement is observed upon supplementation with S1 or S15 beyond the effect of S20 and S16. Data reflect the mean and standard deviation of 8 biological replicates. Comprehensive o-translation data reported in FIG. 14 below.

FIG. 12 shows that E. faecalis 16S rRNA helices have low sequence similarity to those of E. coli. E. faecalis and E. coli rRNAs were aligned using Clustal Omega with default parameters (43), and regions with low sequence identity were manually identified. Elements that were later transplanted into the E. coli 16S o-rRNA are identified in blue.

FIGS. 13A to 13N show the sequence similarity between the r-proteins of species evaluated in this study and those of E. coli. R-proteins identified as enhancing o-rRNA activity are highlighted in blue. FIG. 13A shows the sequence similarity between r-proteins of V. cholera and that of E. coli. FIG. 13B shows the sequence similarity between r-proteins of A. macleodii cholera and that of E. coli. FIG. 13C shows the sequence similarity between r-proteins of M. minutulum cholera and that of E. coli. FIG. 13D shows the sequence similarity between r-proteins of P. aeruginosa cholera and that of E. coli. FIG. 13E shows the sequence similarity between r-proteins of A. baumannii cholera and that of E. coli. FIG. 13F shows the sequence similarity between r-proteins of A. faecalis cholera and that of E. coli. FIG. 13G shows the sequence similarity between r-proteins of N. gonorrhoeae cholera and that of E. coli. FIG. 13H shows the sequence similarity between r-proteins of B. pertussis cholera and that of E. coli. FIG. 13I shows the sequence similarity between r-proteins of B. cenocepacia cholera and that of E. coli. FIG. 13J shows the sequence similarity between r-proteins of M. ferroxydans cholera and that of E. coli. FIG. 13K shows the sequence similarity between r-proteins of C. crescentus cholera and that of E. coli. FIG. 13L shows the sequence similarity between r-proteins of R. parkeri cholera and that of E. coli. FIG. 13M shows the sequence similarity between r-proteins of E. faecalis cholera and that of E. coli. FIG. 13N shows the average r-protein sequence similarity to E. coli for species evaluated in this study which were not immediately functional in E. coli prior to r-protein complementation. Protein sequences were identified via BLAST to E. coli sequences (see Example 1 below). Note that in some cases multiple homologs were identified or a full complement of r-proteins was not identified.

FIG. 14 shows a summary of heterologous translation data. Doubling times in SQ171 cells (minutes) and orthogonal translation activity (normalized to orthogonal E. coli) for all heterologous ribosomes tested. “nIS” indicates native intergenic sequences. “EcIS” indicates E. coli intergenic sequences. “NA” indicates not applicable. “NT” indicates not tested. “*” indicates N=1; only one colony was obtained by SQ171 complementation passing Kan counterscreening against pCSacB. Otherwise, data reflect mean and standard deviation of 3-8 biological replicates.

FIG. 15 shows the excitation and emission wavelengths of the fluorescent protein reporters employed infra. Highlighted plasmids have been deposited in Addgene.

FIG. 16 shows the species names and Genome Taxonomy Database (GTDB) representative genomes used to construct the phylogenetic tree of the instant disclosure (see FIG. 2D above).

FIG. 17 provides descriptive information regarding the accompanying Sequence Listing.

FIGS. 18A to 18E show the assessment of E. coli and heterologous ribosome subunit association using the erythromycin-dependent reporter system. FIG. 18A shows the development of the erythromycin-resistant E. coli strain S4246. All 7 rrl (A-H) 23S rRNA genes were mutated (A2058U) via oligonucleotide recombineering to endow high erythromycin resistance (ERY; MIC >1000 μg mL⁻¹). FIG. 18B shows a schematic representation of the ERY-dependent sfGFP reporter. In the absence of ERY, sfGFP is efficiently translated via orthogonal translation. Addition of ERY (100 μg mL⁻¹) promotes translation stalling at ermCL, abrogating sfGFP translation by ERY-sensitive LSUs. FIG. 18C shows that free and stapled ERY-sensitive LSUs showed a marked reduction in sfGFP production at high inhibitor concentrations, whereas the corresponding ERY-resistant (23S A2058U) LSUs showed no appreciable change in activity (n=3). FIG. 18D shows that ERY-sensitive LSUs re-established strain sensitivity to ERY due to free subunit exchange between episomally- and genomically-derived ribosomes (n=21). FIG. 18E shows an evaluation of intersubunit exchange using the ERY-dependent reporter system. Heterologous ribosomes with high 16S sequence identity to E. coli (≥99.2%) appeared to freely exchange with host subunits, while heterologous ribosomes with intermediate sequence identity (97.0-92.9%) preferentially associated with cognate subunits at a rate comparable to the stapled E. coli ribosome. More divergent heterologous ribosomes (90.3-79.3%) preferentially utilized E. coli large subunits (n=28 for E. coli; otherwise n=7). Data for each ribosome has been normalized to its corresponding sfGFP signal at 0 μg mL⁻¹ ERY. Data reflect the mean and standard deviation of the indicated biological replicates. Comprehensive data are also reported in FIG. 21 below.

FIGS. 19A and 19B show a comparison of SQ strain complementation and orthogonal translation. FIG. 19A shows that to evaluate heterologous rRNAs via SQ strain complementation, rRNA plasmids were transformed into the SQ171 strain. After transformation, colonies took up to 120 hours to form. Colonies were then grown in media+/−kanamycin, and were evaluated over 3 days for pSacB persistence, after which colonies were glycerol stocked. Finally, colonies were grown overnight and back-diluted to generate a growth curve. FIG. 19B shows that to evaluate heterologous rRNAs via orthogonal translation, rRNA plasmids were transformed alongside the reporter plasmid and colonies were incubated overnight. Colonies were then picked into media and grown overnight, after which sfGFP fluorescence was read. Detailed experimental conditions for both assays are described in Example 1 below.

FIGS. 20A to 20E show benchmarking of the ERY-dependent reporter system. FIG. 20A shows that the ERY-dependent reporter discriminated between three possible subunit assembly scenarios. When an orthogonal SSU assembled with a cognate LSU, the ribosome was unable to translate the orthogonal sfGFP reporter due to ERY-sensitivity. Alternatively, heterologous SSUs may assemble with E. coli LSUs, resulting in robust sfGFP translation. Finally, E. coli SSUs may assemble with heterologous LSUs, resulting in strain toxicity due to an inability to translate essential E. coli genes and low sfGFP signal as a result. FIG. 20B shows that heterologous ribosomes closely related to E. coli (>99.2% 16S sequence ID) re-sensitized S4246 cells to ERY treatment due to the usage of sensitive LSUs for translating host genes (n=7). FIG. 20C shows orthogonal translation activities observed for native ribosomes and ribosomes stapled to cognate LSUs vs. E. coli LSUs (n=8). FIG. 20D shows ERY-dependent reporter data obtained for native ribosomes and ribosomes stapled to cognate LSUs vs. E. coli LSUs at 100 μg mL⁻¹ ERY. Data for each ribosome was normalized to its sfGFP fluorescence at 0 μg mL⁻¹ ERY (n=28 for E. coli; otherwise n=7). FIG. 20E shows that OD600 values observed for heterologous ribosomes with high 16S sequence identity to E. coli (≥99.2%) at 100 μg mL⁻¹ ERY increased after subunit stapling, which indicated a decrease in intersubunit exchange (n=7). Data reflect means and standard deviations of the indicated biological replicates. Comprehensive data are reported in FIG. 21 below.

FIG. 21 shows a summary of stapled ribosome data. Data reflect means and standard deviations of n=7-28 biological replicates.

FIG. 22 shows a list of rRNA and r-protein expression plasmids used in certain embodiments of the instant disclosure. The rRNA/r-protein combinations that yielded the highest degree of activity have been highlighted in the figure and have also been deposited in Addgene.

### DETAILED DESCRIPTION OF THE INVENTION

The present disclosure is directed, at least in part, to discovery of an improved system for monitoring and improving heterologous ribosome activity within a host cell (optionally, within a highly tractable host cell, such as E. coli, B. subtilis, or other), where such assessment is not reliant upon the heterologous ribosome maintaining growth of the host cell. Specifically, the instant disclosure provides a reporter system that enables monitoring of heterologous ribosome activity in a host cell, via engineering of heterologous rRNA operon sequences and reporter operon sequences. Strikingly, for certain heterologous ribosomes, activity of a heterologous rRNA operon can be improved in a host cell by replacing intergenic sequences of the heterologous operon with corresponding host cell intergenic sequences, as well as via introduction of certain heterologous r-proteins (e.g., S20, S16, S1 and/or S15 r-proteins).

Various new heterologous rRNA-harboring genetic organisms have also been provided herein. Such organisms allow for improved screening approaches to identify ribosome-targeting agents, as well as for improved synthetic ribosome evolution. The instant disclosure therefore also provides methods and compositions for identifying ribosome-targeting antibiotic agents, including, e.g., identification of narrow-spectrum antibiotic agents that preferentially target ribosomes of pathogenic microbes, as compared to ribosomes of commensal microbes (in certain embodiments, ribosomal components of pathogenic microbes and commensal microbes can be multiplexed within a common host cell, allowing for direct comparison of, e.g., response to a test compound, between ribosomal components of pathogenic microbes and those of commensal microbes). Such narrow-spectrum antibiotic agents promise clear therapeutic advantages, e.g., where applied to microbes of the gut microbiome, as well as in other scenarios. The compositions, methods and application(s) of the instant disclosure are considered in additional detail below.

The ribosome represents a promising avenue for synthetic biology, but its complexity and essentiality have until now hindered significant engineering efforts. Heterologous ribosomes, comprising rRNAs from divergent organisms, are expected to offer opportunities for enhanced orthogonality or discovery of novel translational functions. Such ribosomes have previously been evaluated in E. coli via complementation of a genomic ribosome deficiency, but this prior art method has failed to guide engineering of refractory ribosomes. In certain aspects, the instant disclosure has implemented orthogonal ribosome binding site (RBS):anti-RBS pairs to quantify the translation of heterologous ribosomes, which has been observed herein to significantly improve the accuracy and throughput of heterologous ribosome analysis. Orthogonal translation has been applied herein to define general requirements for efficient heterologous rRNA processing, and the instant disclosure has discovered that supplementation with a small subset of cognate r-proteins enhanced heterologous ribosome activity for rRNAs derived from organisms with as little as 76.1% 16S rRNA sequence identity to E. coli. In addition, the instant disclosure has identified that moderately divergent heterologous rRNAs can selectively assemble into species-specific ribosomes with limited E. coli subunit association. Cumulatively, certain aspects of the instant disclosure provide a general framework for heterologous ribosome engineering in living cells.

Heterologous ribosomes are canonically produced in E. coli via introduction of ribosomal components derived from extant microbes. Until now, the efficiency of this process has not been high for increasingly divergent microbes, owing to poor ribosome maturation and interaction with host E. coli factors. The instant disclosure has identified defined methods for producing previously intractable heterologous ribosomes in E. coli via (1) processing signal engineering and (2) ribosomal protein complementation, thereby yielding high activity heterologous ribosomes that mimic the natural counterparts.

In particular, non-native ribosomes (i.e., ribosomes from foreign organisms) have been employed herein, and systems for improving heterologous ribosome function in E. coli have been engineered through the following: (1) engineering of specific intergenic sequences to enhance and enable robust ribosomal RNA maturation via interaction with appropriate cellular RNAses, and (2) supplementation with select cognate ribosomal proteins to complement assembly deficiencies of heterologous rRNAs.

In addition to catalyzing the biosynthesis of the complete cellular proteome, the ribosome serves as a hub for signaling events, integrating nutrient availability with growth dynamics and resource allocation (1, 2). In prokaryotes, this functionality is enabled by the concerted action of numerous components: the 16S rRNA and 21 ribosomal proteins (r-proteins) define the small subunit (SSU or 30S), whereas the 23S rRNA, 5S rRNA and 33 r-proteins define the large subunit (LSU or 50S) (3). Extensive efforts towards engineering translation have yielded researcher-dictated, specialized functions in vivo: parallel genetic circuits (4, 71-74), augmented polypeptide diversity using non-canonical amino acids (5), expanded genetic codes incorporating quadruplet codons (6), and linked ribosomal subunits for improved cellular orthogonality (7-9). However, these efforts typically made use of E. coli components, yielding systems that continue to crosstalk with the chassis bacterial cell in unpredictable ways.

Conversely, ribosomal components derived from divergent microorganisms offer enhanced orthogonality as well as the opportunity to discover unique or dedicated ribosomal capabilities, as indicated by naturally occurring subpopulations of prokaryotic ribosomes (10): stress-inducible production of rrsH ribosomes in E. coli modifies the cellular translational program (11), rrnl ribosomes in Vibrio vulnificius selectively translate certain mRNAs (70), and genetically heterogeneous ribosomes are produced at defined stages of the complex Streptomyces coelicolor developmental cycle (12). R-protein complements further specialize ribosomal function, as they are known to vary in the E. coli ribosome under different growth conditions (13), and ribosomes carrying S1 play a role in leaderless mRNA decoding (14). Heterologous ribosomes, synthesized in E. coli using diverse rRNAs and cognate r-proteins, therefore facilitate the discovery or engineering of novel translational capabilities.

Prior investigations of heterologous rRNAs have employed E. coli Δ7 strains lacking all seven chromosomal rRNA operons (e.g., SQ171, KT101, SQZ10, SQ2518) (15-17). These Δ7 strains additionally informed studies on rDNA copy number (18), ribosomal sequence-function relationships (19), factors affecting rRNA processing (20, 21), and rRNA-protein interactions (22). These strains bear a complete genomic rRNA deficiency and are complemented by a counter-selectable rRNA-encoding plasmid, facilitating plasmid exchange with rRNA variants capable of sustaining E. coli survival. Indeed, full-length heterologous rRNA operons derived from species bearing ≥93.2% 16S sequence identity to their E. coli counterparts were found to sustain E. coli Δ7 strain viability (17), whereas 16S sequence fragments bearing ≥80.9% identity were identified to substitute for otherwise wildtype E. coli 16S rRNAs (16). Natural horizontal gene transfer events in the evolutionary record have provided further evidence for heterologous translation with intragenomic 16S identity as low as 88.4% (23-27).

However, past E. coli Δ7 strain complementation assays have proven problematic for systematically evaluating heterologous ribosome function given the myriad roles played by the ribosome in sustaining cell viability, both catalytic and regulatory (28, 29). Specifically, past efforts to engineer heterologous ribosome function have been hampered by the absence of a more quantitative translational assay that reports on a single aspect of ribosome function (as is now disclosed herein). Therefore, guidelines have now been developed herein for evaluating and enhancing the translational activity of heterologous rRNAs in E. coli using a method that reports exclusively on catalytic activity, independent of an rRNA's ability to support cell growth. To achieve this, a library of 34 complete rRNA operons derived from phylogenetically diverse microbes was constructed. Activities of the of 34 complete rRNA operons have been evaluated herein via E. coli Δ7 strain complementation as well as orthogonal translation, which implemented engineered RBS:anti-RBS pairs that exclusively translated researcher-defined reporter transcripts (4, 71-74). Finding a high degree of correlation between the two methods, the orthogonal translation method has been applied herein to guide rRNA processing sequence engineering. The instant disclosure has therefore identified that divergent intergenic sequences exert significant consequences upon heterologous ribosome maturation in E. coli. Furthermore, a small subset of r-proteins were identified that significantly enhanced the activity of refractory heterologous ribosomes possessing as little as 76.1% 16S rRNA sequence identity to E. coli. In addition, it has been identified herein that heterologous 16S rRNAs having intermediate sequence identity preferentially associated with their cognate 23S rRNAs in E. coli. Together, these results have established the herein-disclosed quantitative and extensible method(s) for the engineering of heterologous ribosome activity in vivo, which greatly facilitates the development of diverse ribosomes for synthetic biology applications (including the development of diverse ribosomes with specialized functions). In addition to the process for improving heterologous rRNA maturation and activity, new organisms harboring heterologous rRNAs have also been manufactured and provided herein. Use of such new organisms to streamline identification of new antibiotics, including highly selective/narrow spectrum antibiotics capable of selectively killing targeted pathogenic microbes, is also contemplated.

Herein, microbial phylogenetic relationships have been integrated with orthogonal translation via engineered ribosome binding site (RBS):anti-RBS pairs to quantify heterologous ribosome activity. The findings disclosed herein highlight generally applicable requirements for efficient rRNA processing and cognate r-protein supplementation, which yield functional heterologous ribosomes in E. coli. (and are contemplated to yield functional heterologous ribosomes in other microbes (e.g., B. subtilis) Cumulatively, the instant disclosure also enables further generation of functional heterologous ribosomes possessing new and specialized capabilities for synthetic translation.

rRNAs and r-Proteins

The E. coli ribosome is composed of two large particles, the 30S and the 50S subunits. The 30S subunit consists of a 16S rRNA molecule and 21 small ribosomal proteins (“r-proteins’). The 50S subunit is composed of two ribosomal RNA (rRNA) molecules, 23S and 5S rRNA, and 31 large ribosomal proteins.

Prokaryotic ribosomes are similar across species, but homology of individual ribosomal proteins diverges with phylogenetic distance. rRNAs are relatively few in number and yet play an important role in protein synthesis (Gutell et al., 1985, Prog. Nucleic Acid Res. Mol. Biol. 32:155-216). Ribosome assembly in bacteria is a tightly controlled process. For example, the synthesis of ribosomal components, rRNA and r-proteins, is coordinately regulated to ensure that these molecules are produced in the optimal stoichiometry. Protein-RNA interactions play important regulatory roles at several steps in this process. Synthesis of r-proteins is negatively regulated at the translational level by the binding of repressor r-proteins to specific sites in mRNA. As part of another regulatory step in the ribosome assembly process, certain r-proteins bind to rRNA, to initiate the ordered assembly of the ribosome. Binding of these r-proteins, termed “primary binders,” is required for the subsequent steps of ribosome assembly (Zengel & Lindahl, 1994, Prog. Nuc. Acid Res. Molec. Biol. 47:332-370).

The interaction of ribosomal proteins with RNA influences the synthesis of ribosomal proteins and their assembly into fully functional ribosomes. Ribosomal assembly in E. coli involves the coordinate expression of rRNA and r-proteins. Binding of certain ribosomal proteins to ribosomal RNAs (rRNAs) is necessary for the ordered assembly of fully functional ribosomes. In the course of assembly, a subset of ribosomal proteins, termed primary binding r-proteins, has been identified as binding rRNA directly, and as facilitating the binding of other ribosomal proteins.

rRNA, r-Protein and Construct Sequences

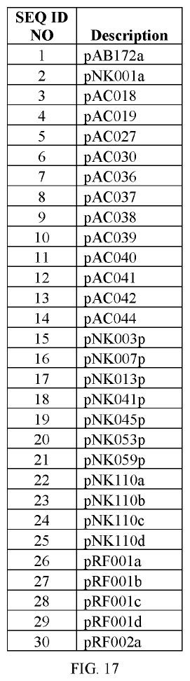

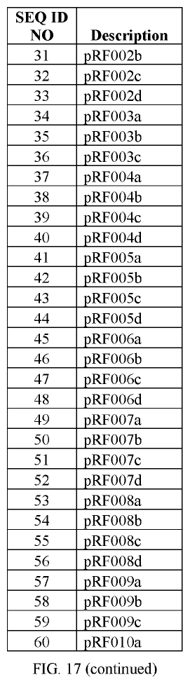

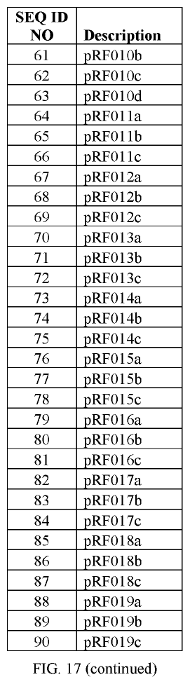

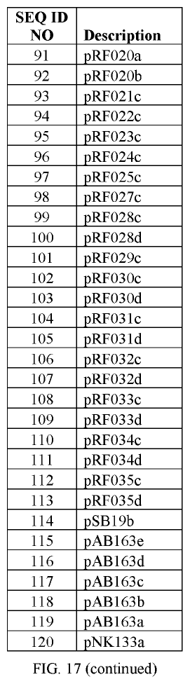

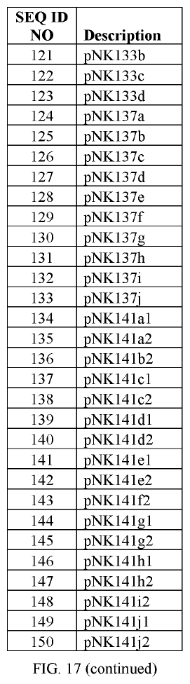

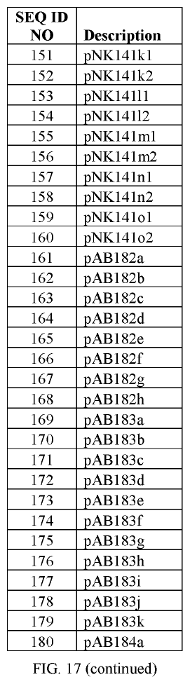

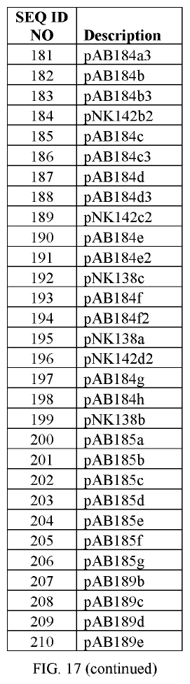

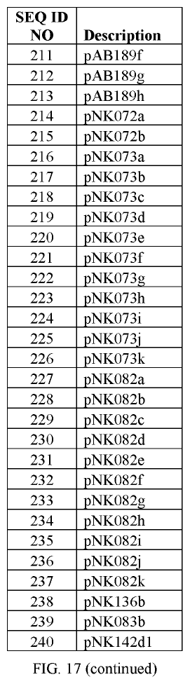

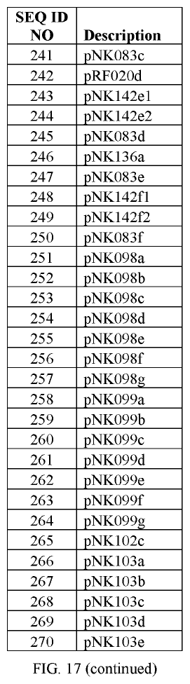

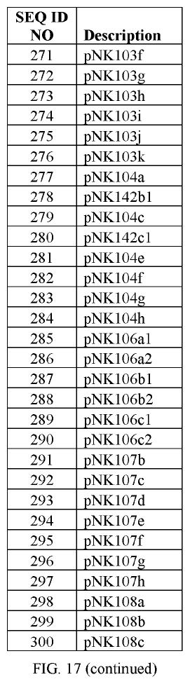

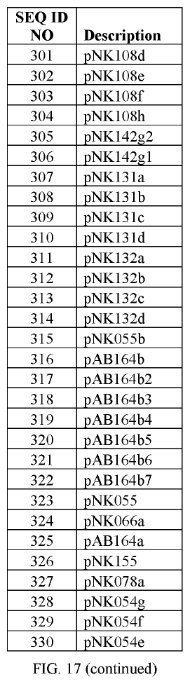

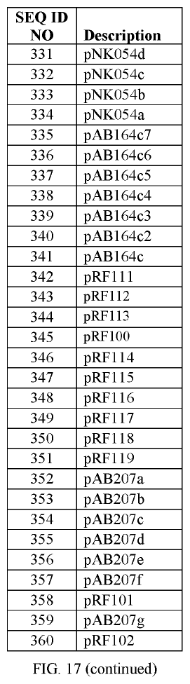

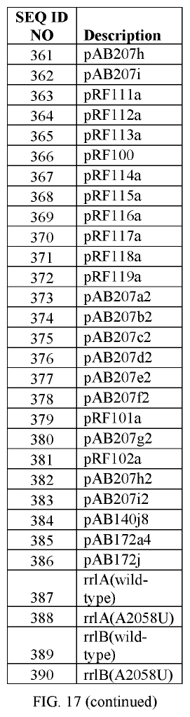

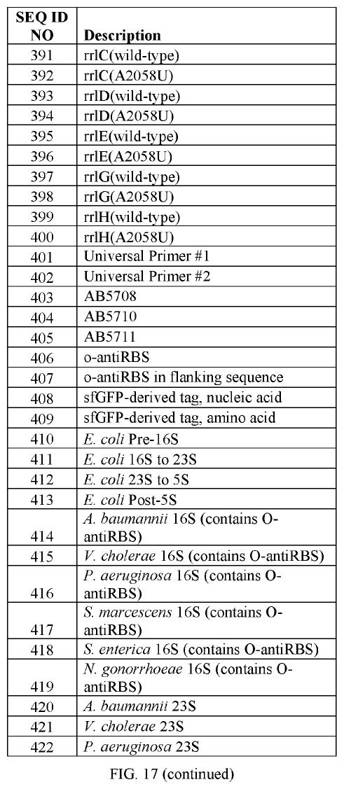

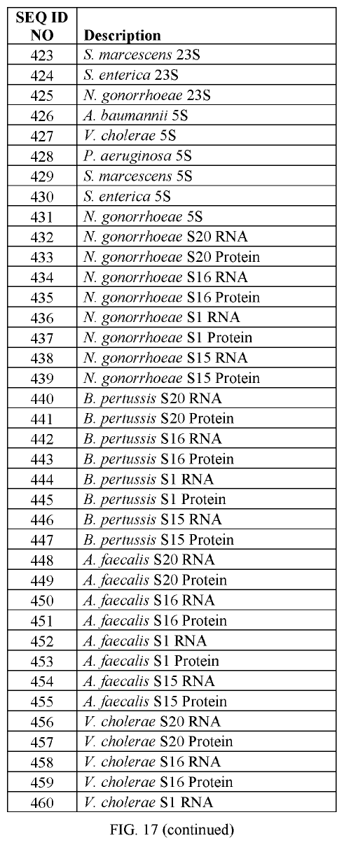

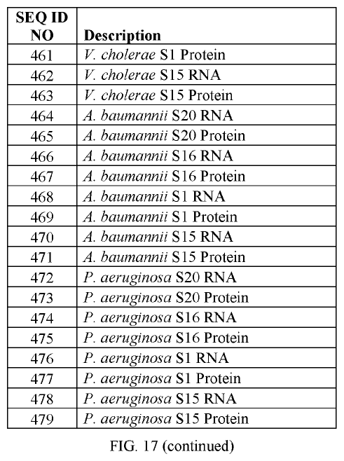

rRNA, r-protein and other rRNA and/or reporter construct sequences of the instant disclosure are presented in the accompanying Sequence Listing, with FIG. 17 also presenting a description of each sequence.

rRNA-Deleted Host Strains

It is expressly contemplated that certain compositions and methods of the instant disclosure can employ any appropriate rRNA-deleted host cell. Exemplary rRNA-deleted host cells include, without limitation:

SQ171 is an rrn E. coli strain lacking all seven chromosomal rRNA operons and carrying a single, counter-selectable plasmid bearing the wildtype rrnC operon (50).

KT101 is another example of a rrn E. coli strain lacking all seven chromosomal rRNA operons (rrnA, B, C, D, E, G, H) (21). Growth of KT01 can be complemented by the rrnB operon encoded in rescue plasmid pRB101 (Kitahara et al. PNAS 109: 19220-19225).

### Bacterial Culture and Transformation

Culture and transformation of bacterial cells can be performed by any art-recognized method. E. coli is commonly propagated in rich media, with examples including LB, 2× yeast extract-tryptone (YT), Terrific Broth (TB), and Super Broth (SB).

While early attempts to achieve transformation of E. coli were unsuccessful and it was at one time even believed that E. coli was refractory to transformation, Mandel and Higa (J. Mol. Bio. 53: 159-162 (1970)) found that treatment with CaCl₂) allowed E. coli bacteria to take up DNA from bacteriophage k. In 1972, Cohen et al. showed CaCl₂)-treated E. coli bacteria were effective recipients for plasmid DNA (Cohen et al., Proc. Natl. Acad. Sci., 69: 2110-2114 (1972)). Since transformation of E. coli is an essential step or cornerstone in many cloning experiments, it is desirable that it be as efficient as possible (Lui and Rashidbaigi, BioTechniques 8: 21-25 (1990)). Hanahan (J. Mol. Biol. 166: 557-580 (1983), herein incorporated by reference) examined factors that affect the efficiency of transformation, and devised a set of conditions for optimal efficiency (expressed as transformants per μg of DNA added) applicable to most E. coli K12 strains. Typically, efficiencies of 10⁷ to 10⁹ transformants/μg can be achieved depending on the strain of E. coli and the method used (Liu & Rashidbaigi, BioTechniques 8: 21-25 (1990), herein incorporated by reference).

Many methods for bacterial transformation are based on the observations of Mandel and Higa (J. Mol. Bio. 53: 159-162 (1970)). Apparently, Mandel and Higa's treatment induces a transient state of “competence” in the recipient bacteria, during which they are able to take up DNAs derived from a variety of sources. Many variations of this basic technique have since been described, often directed toward optimizing the efficiency of transformation of different bacterial strains by plasmids. Bacteria treated according to the original protocol of Mandel and Higa yield 10⁵-10⁶ transformed colonies/μg of supercoiled plasmid DNA. This efficiency can be enhanced 100- to 1000-fold by using improved strains of E. coli (Kushner, In: Genetic Engineering: Proceedings of the International Symposium on Genetic Engineering, Elsevier, Amsterdam, pp. 17-23 (1978); Norgard et al., Gene 3:279-292 (1978); Hanahan, J. Mol. Biol. 166: 557-580 (1983)) combinations of divalent cations ((Kushner, In: Genetic Engineering: Proceedings of the International Symposium on Genetic Engineering, Elsevier, Amsterdam, pp. 17-23 (1978)) for longer periods of time (Dagert and Ehrlich, Gene 6: 23-28 (1979)) and treating the bacteria with DMSO (Kushner, In: Genetic Engineering: Proceedings of the International Symposium on Genetic Engineering, Elsevier, Amsterdam, pp. 17-23 (1978)), reducing agents, and hexamminecobalt chloride (Hanahan (J. Mol. Biol. 166: 557-580 (1983).

A number of procedures exist for the preparation of competent bacteria and the introduction of DNA into those bacteria. A very simple, moderately efficient transformation procedure for use with E. coli involves re-suspending log-phase bacteria in ice-cold 50 mM calcium chloride at about 10¹⁰ bacteria/ml and keeping them ice-cold for about 30 min. Plasmid DNA (0.1 mg) is then added to a small aliquot (0.2 ml) of these now competent bacteria, and the incubation on ice continued for a further 30 min, followed by a heat shock of 2 min at 42° C. The bacteria are then usually transferred to nutrient medium and incubated for some time (30 min to 1 hour) to allow phenotypic properties conferred by the plasmid to be expressed, e.g. antibiotic resistance commonly used as a selectable marker for plasmid-containing cells. Protocols for the production of high efficiency competent bacteria have also been described and many of those protocols are based on the protocols described by Hanahan (J. Mol. Biol. 166:557-580 (1983).

Another rapid and simple method for introducing genetic material into bacteria is electoporation (Potter, Anal. Biochem. 174: 361-73 (1988)). This technique is based upon the original observation by Zimmerman et al., J. Membr. Biol. 67: 165-82 (1983), that high-voltage electric pulses can induce cell plasma membranes to fuse. Subsequently, it was found that when subjected to electric shock (typically a brief exposure to a voltage gradient of 4000-16000 V/cm), the bacteria take up exogenous DNA from the suspending solution, apparently through holes momentarily created in the plasma membrane. A proportion of these bacteria become stably transformed and can be selected if a suitable marker gene is carried on the transforming DNA transformed (Newman et al., Mol. Gen. Genetics 197: 195-204 (1982)). With E. coli, electroporation has been found to give plasmid transformation efficiencies of 10⁹-10¹⁰/μg DNA (Dower et al., Nucleic Acids Res. 16: 6127-6145 (1988)).

Bacterial cells are also susceptible to transformation by liposomes (Old and Primrose, In Principles of Gene Manipulation: An Introduction to Gene Manipulation, Blackwell Science (1995)). A simple transformation system has been developed which makes use of liposomes prepared from cationic lipid (Old and Primrose, (1995)). Small unilamellar (single bilayer) vesicles are produced. DNA in solution spontaneously and efficiently complexes with these liposomes (in contrast to previously employed liposome encapsidation procedures involving non-ionic lipids). The positively-charged liposomes not only complex with DNA, but also bind to bacteria and are efficient in transforming them, probably by fusion with the cells. The use of liposomes as a transformation or transfection system is called lipofection.

B. subtilis (as well as other microbes) can be grown in culture via art-recognized methods. Transformation of B. subtilis can be achieved via methods including electroporation, protoplast transformation (B. subtilis protoplasts can be transformed but are fragile, with only about 1-10% of protoplasts surviving transformation and becoming regenerated) and use of natural competence, among other methods (see, e.g., Zhang and Zhang. Microb. Biotechnol. 4: 98-105).

### Pathogenic Microbes

Contemplated pathogenic microbes of the instant disclosure include, without limitation, bacteria from the following genera: Bordetella, Borrelia, Brucilla, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio and Yersinia.

In certain embodiments, the pathogenic microbe is a bacteria or bacterial combination selected from among the following: Mycobacterium tuberculosis, Bifidobacterium longum, Veillonella parvula, Clostridium difficile, Bacillus subtilis, Escherichia coli, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica, Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris, Haemophilus influenza and Shigella spp. (e.g., Shigella flexneri, Shigella dysenteriae, Shigella sonnei, Shigella boydii).

### Commensal Microbes

Contemplated commensal microbes of the instant disclosure include, without limitation, microbes of the phyla Firmicutes, Bacteroidetes, Bifidobacteria, Eubacteria, Ruminococcus, Lactobacillus, Peptococcus, Proteobacteria, Verrumicrobia, Actinobacteria, Fusobacteria and/or Cyanobacteria, as well as combinations thereof.

### Test Compound Libraries/Compound Screening

Certain embodiments of the instant disclosure expressly contemplate identification of heterologous ribosome-targeting agents, including in certain embodiments agents that are selective for ribosomes of pathogens, optionally as compared to ribosomes of commensal microorganisms. Exemplary forms of such agents include small molecules and macromolecules (e.g., peptides, antibodies, nucleic acids and other biologics). For example, screening of a small molecule drug-repurposing library can be performed to identify agents that selectively inhibit ribosomes derived from pathogenic organisms. Specific examples of expressly contemplated compound libraries include the Pharmakon library (www.msdiscovery.com/pharmakon.html), other Microsource libraries (US Drug collection, NatProd collection or Spectrum collection), Chembridge screening libraries (EXPRESS-Pick Collection Stock or CORE Library Stock; www.chembridge.com/screening_libraries/), HTS, Advanced, and Premium screening libraries from Enamine (enamine.net/hit-finding/compound-collections/screening-collection), among other available libraries.

### Kits

The instant disclosure also provides kits containing agents of this disclosure for use in the methods of the present disclosure. Kits of the instant disclosure may include one or more containers comprising a nucleic acid construct, organism, or other component of the system(s) described herein.

The instructions generally include information as to use of the components included in the kit. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

Unless otherwise noted, all PCRs were performed using Phusion U HotStart DNA Polymerase (Life Technologies). Water was purified using a MilliQ water purification system (MilliporeSigma). Antibiotics (Gold Biotechnology) were used at the following concentrations for plasmid selection: 30 μg mL⁻¹ Kanamycin, 40 μg mL⁻¹ chloramphenicol, 50 μg mL⁻¹ carbenicillin, 100 μg mL⁻¹ spectinomycin. Antibiotics concentrations were one-third the aforementioned for strains bearing three unique plasmids. Unless otherwise noted, all DNA manipulations were performed in NEB Turbo cells (New England Biolabs) or Mach1F cells (Mach1 T1_{R} cells (Thermo Fisher Scientific) mated with F′ episome of the previously described S2060 strain (47)). All fluorescence and luminescence assays were carried out using E. coli S2060 (47).

### Chemically Competent Cell Preparation

Chemically competent cells were prepared for cloning and assay strains. A glycerol stock of the appropriate strain was used to start a 2 mL culture of the strain supplemented with the appropriate antibiotics and grown up overnight at 30° C. at 300 RPM. The saturated culture was diluted 1:1000 in 50 mL 2×YT (United States Biological) with appropriate antibiotics and grown to OD=0.3-0.5 in a 370 shaker at 300 RPM. Cells were pelleted in a pre-chilled conical tube (VWR) by centrifugation at 8,000 g for 10 min at 4° C. The supernatant was removed and the cells were resuspended in approximately 20 mL 10% glycerol, then pelleted by centrifugation at 8,000 g for 10 min at 4° C. The supernatant was removed and the cells were resuspended in chilled TSS buffer (2×YT media supplemented with 5% DMSO, 10% PEG2250, 2 mM MgCl2) Cells were flash frozen in liquid N₂ at 100 μL aliquots and transferred to −80° C. storage.

### USER Cloning

Plasmids were constructed using USER cloning, or a combination of USER cloning and overlap extension PCR. In USER cloning, primers are designed to include a deoxyuracil base approximately 10-20 from the 5′ end of the primer; the region between the deoxyuracil base and the 5′ end of the primer is known as the “USER junction” and specifies the homology necessary for plasmid assembly. USER junctions were designed to have a 42° C.<Tm<70° C., minimal secondary structure, and begin with a dA and end with a dT (the latter is replaced with a dU by uracil DNA glycosylase during assembly). PCR products were all gel purified using QIAquick Gel Extraction kit (Qiagen) and eluted to a final volume of 10 μL. Fragments were quantified using a NanoDrop 1000 Spectrophotometer (Thermo Fisher Scientific). For assembly, PCR products with complementary USER junctions were added in an equimolar ratio (0.1-1 pmol each) in a 10 μl reaction containing 0.75 units DpnI (New England Biolabs), 0.75 units USER (Uracil-Specific Excision Reagent; Endonuclease VIII and Uracil-DNA Glycosylase) enzyme (New England Biolabs), 1 unit of CutSmart Buffer (50 mM potassium acetate, 20 mM Tris-acetate, 10 mM magnesium acetate, 100 μg mL⁻¹ BSA at pH 7.9; New England Biolabs). Reactions were incubated at 370 for 20 min, then heated at 80° C. and slowly cooled to 22° C. at 0.1° C./s in a thermocycler.

Inserts of plasmids “A01”, “AO2”, and “S1-S21,” consisting of many small fragments, were cloned using overlap extension PCR. Primers were designed containing ˜15 bp overhangs complementary to the adjoining fragment. Individual fragments were amplified and gel purified as above; then 0.2 picomoles of each fragment was used in a 200 μL PCR reaction to join each fragment together. This fragment was gel purified and USER assembly was used to clone it into the plasmid backbone. Plasmid constructs were heat-shocked into chemically competent NEB Turbo or Mach1f cells: 100 μL 2×KCM (100 mM KCl, 30 mM CaCl₂), 50 mM MgCl2 in MilliQ H2O) was added to 100 μl cells alongside plasmid DNA. Cells were incubated on ice for 15 min, heat shocked at 42° C. for 2 min, and placed back on ice for 2 min. Cells were recovered in 1 mL 2×YT media at 37° with shaking at 300 RPM for a minimum of 45 min. Cells were streaked on 2×YT media+1.8% agar plates supplemented with the appropriate antibiotic.

### Amplification of Ribosomal Operons and R-Proteins

Ribosomal DNA, contiguous r-protein operons and single r-protein ORFs were amplified from bacterial strains or the corresponding gDNA. Direct amplification from bacterial strains required boiling at 95° C. for 10 min in MilliQ water prior to PCR for efficient amplification. In cases where a non-type strain was used, universal primers

(5′-ACCGGCCGCUgtgccagcagccgcggtaatac-3′,
SEQ ID NO: 401)
and
(5′-AGGGGTTCCGCGCACAUgtgacgggcggtgtgtacaag-3′,
SEQ ID NO: 402)
(USER junctions bold, annealing region is
underlined)

were used to amplify a ˜900 bp fragment from the bacterial genome to include a partial 16S element for subcloning and sequencing, allowing for closest sequenced genome determination. For species with high sequence variability between ribosomal operons, a representative operon was chosen based on maximal sequence homology to E. coli.

### Bacterial Strain Genomic Modifications

The erythromycin-resistant strain S4246 was generated using conventional recombineering (76, 77). Briefly, chemically competent S2060 cells were transformed with pKD46 (77) and plated on 2×YT agar plates supplemented with 50 μg mL⁻¹ carbencillin at 30° C. A single colony was picked, grown at 30° C. in 2×YT liquid medium supplemented with 50 μg mL⁻¹ carbencillin and 10 mM arabinose, and made chemically competent when the culture reached the appropriate OD600. Chemically competent S2060/pKD46 cells were transformed with the phosphothiorated recombineering oligonucleotide AB5708 (5′-C*T*C*A*ATGTTCAGTGTCAAGCTATAGTAAAGGTTCACGGGGTCTTACCGTCTTG CCGCG GGTACACTGCATCTTCACAGCGAGTTCAATTT-3′, SEQ ID NO: 403; phosphothirate bonds indicated as *, introduced mutation is bolded) to introduce the rrlA-H A2058U mutation on replichore 2 (76). Following recovery for 3 hours at 30° C., transformed cells were plated on 2×YT agar plates supplemented with 1000 μg mL⁻¹ erythromycin and incubated at 37° C. to cure the resident pKD46 plasmid. Following overnight growth, single colonies were picked into 2×YT liquid medium supplemented with 50 μg mL⁻¹ streptomycin, 10 μg mL⁻¹ tetracycline and 1000 μg mL⁻¹ erythromycin and allowed to grow overnight at 37° C. To assess the degree of rrlA-H mutagenesis, cultures were used as PCR templates using primers AB5710 (5′-GAAATTCCTTGTCGGGTAAGTTCC-3′, SEQ ID NO: 404) and AB5711 (5′-GAACATCAAACATTAAAGGGTGGTATTTC-3′, SEQ ID NO: 405), and the PCR products were treated with the endonuclease HpyCH4III (New England Biolabs) according to the manufacturer's guidelines. Wildtype rrlA-H genotypes show no digestion under these conditions, whereas complete conversion results in complete PCR product digestion. Intermediate (incomplete) digestion indicated in incomplete conversion of all seven genomic alleles. The completely converted strain S4246 was confirmed to be sensitive to the following antibiotics (ensures no resistance crosstalk with plasmid-borne markers): carbenicillin (50 μg mL⁻¹), spectinomycin (100 μg mL⁻¹), chloramphenicol (40 μg mL⁻¹), and kanamycin (30 μg mL⁻¹). The strain was confirmed to be resistant to the following antibiotics: streptomycin (50 μg mL⁻¹), tetracycline (10 μg mL⁻¹), and erythromcyin (1000 μg mL⁻¹).

### Fluorescence Assays

For orthogonal translation assays, S2060 chemically competent cells were transformed with the E. coli o-rRNA plasmid and the relevant orthogonal reporter plasmid. Transformants were streaked on 2×YT media+1.8% agar supplemented with kanamycin and carbenicillin. Plates were grown in a 37° C. incubator for 16 hours. Colonies were picked into DRM (United States Biological) (48) supplemented with kanamycin, carbenicillin, 1 mM IPTG+/−1000 ng/mL aTc. To assay heterologous o-rRNA function, chemically competent cells carrying the sfGFP reporter plasmid were prepared (52060.sfGFP) and transformed with the appropriate o-rRNA plasmid. E. coli o-rRNA was always transformed alongside experimental o-rRNAs as a positive control. Transformants were streaked out and picked into media as above.

To assay r-protein effects on heterologous o-rRNA function, 52060.sfGFP chemically competent cells were co-transformed with the appropriate o-rRNA plasmid and r-protein plasmid. As a positive control, E. coli o-rRNA was transformed alongside an mCherry expression plasmid. In the absence of r-protein supplementation, heterologous o-rRNAs were transformed with mCherry to maintain consistent growth rates and antibiotic selection markers. Transformants were streaked on 2×YT media+1.8% agar supplemented with kanamycin, carbenicillin, chloramphenicol, and 200 mM glucose and picked into DRM supplemented with kanamycin, carbenicillin, chloramphenicol, 1 mM IPTG, 1000 ng/mL aTc, +/−10 mM arabinose.

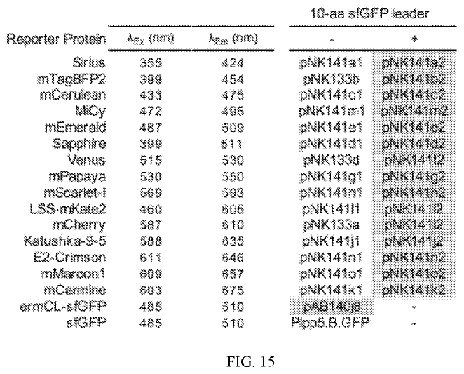

To quantify fluorescence output, 150 μl each culture was aliquoted into a 96-well black wall, clear bottom plate (Costar). OD₆₀₀ and the appropriate excitation and emission wavelengths were used for fluorescence measurements (FIG. 15) using either a SpectraMax M3 (Molecular Devices) or Spark (Tecan) plate reader. Fluorescence was normalized to OD600 after blank media subtraction. Data were normalized to E. coli o-rRNA sfGFP/OD600 and expressed as a percentage; when assaying the effects of r-protein complementation, data were normalized to E. coli o-rRNA sfGFP/OD₆₀₀ bearing the mCherry control plasmid.

### SQ171 Cell Viability Assay

Chemically competent SQ171 (49, 50) cells were transformed with heterologous rRNAs as described above and recovered for up to 7 h in 2×YT in a 37° C. shaker. The recovery culture was centrifuged at 10,000 RCF for 2 min, then the pellet was resuspended in 100 μL MilliQ water. The resuspended cells were diluted serially in seven, 10-fold increments to yield eight total samples (undiluted, 10¹, 10², 10¹, 10⁴, 10⁵, 10⁶, and 10⁷ fold diluted). To determine the efficiencies of EP transformation and counter-selectable plasmid curing, 3 μl of diluted cells were plated on 1.8% agar-2×YT plates (United States Biological) supplemented with spectinomycin (100 μg mL⁻¹) and carbenicillin (50 μg mL⁻¹), with or without 5% sucrose (Millipore Sigma). For picking single colonies, the remaining undiluted cells were plated on 1.8% agar-2×YT plates (United States Biological) containing spectinomycin (100 μg mL⁻¹), carbenicillin (50 μg mL⁻¹) and 5% sucrose. All plates were grown for 16-120 hours in a 37° C. incubator.

Colonies transformed with the appropriate EP and surviving sucrose selection were picked and grown in DRM containing spectinomycin (100 μg mL⁻¹), carbenicillin (50 μg mL⁻¹), and 5% sucrose. Following growth of the EP-carrying strains for up to 3 days, cultures were glycerol stocked. Overnight cultures were started from these glycerol stocks in DRM containing spectinomycin (100 μg mL⁻¹), carbenicillin (50 μg mL⁻¹), and 5% sucrose. Following overnight growth, cultures were diluted 100-fold into fresh DRM containing spectinomycin (100 μg mL⁻¹) and carbenicillin (50 μg mL⁻¹). From the diluted cultures, 200 μl of each culture was transferred to a 96-well black wall, clear bottom plate (Costar), topped with 20 μL of mineral oil, and the OD600 was measured every 5 min over 15 h. Separately, 400 μL of each diluted culture was supplemented with kanamycin (30 μg mL⁻¹) and grown in a 37° C. shaker at 900 RPM. Colonies that survived selection in kanamycin were excluded from final analysis, as survival in kanamycin indicates persistence of the resident pCSacB plasmid (which carries a KanR resistance cassette) The doubling time of each culture was calculated using the Growthcurver package (51) in Rstudio.

### Phylogenetic Analyses

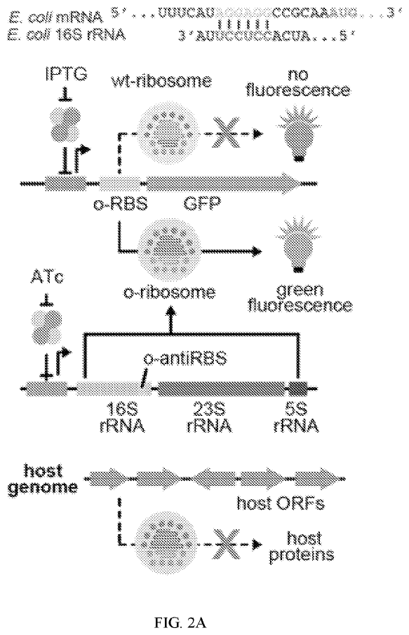

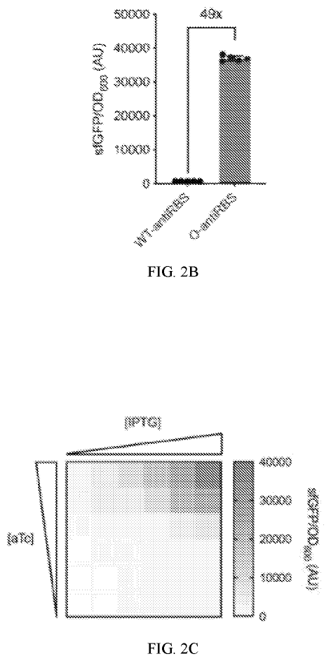

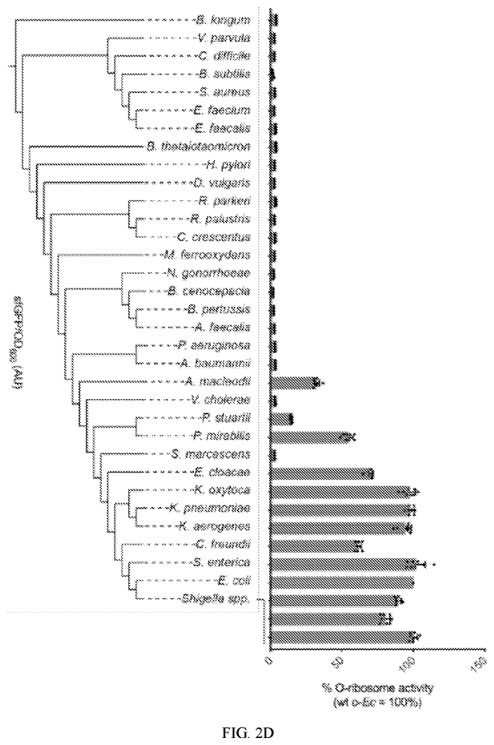

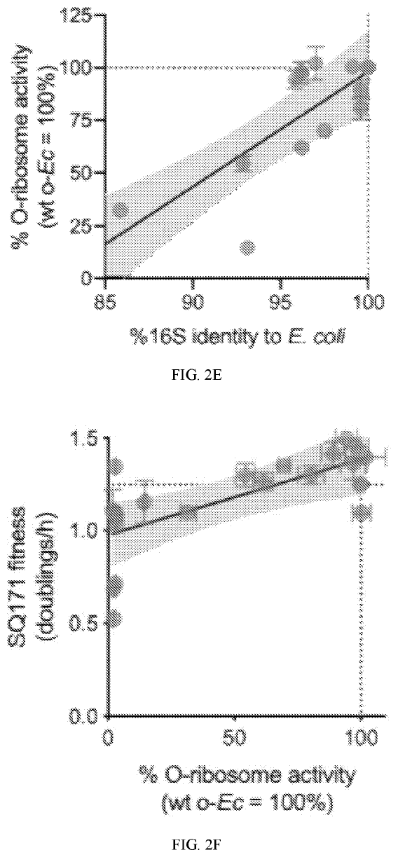

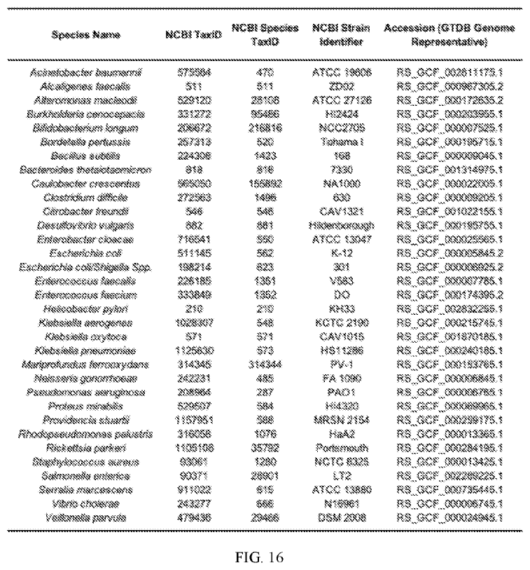

To calculate sequence identities, 16S sequences of all rRNAs used in the study were aligned using Clustal Omega with default settings (43). The phylogenetic tree (FIG. 2D) was constructed using phylogenetic relationships derived from the Genome Taxonomy database (GDTB) (52). In short, the entire bacterial GTDB phylogenetic tree (release 86.1) was downloaded from www.data.ace.uq.edu.au/public/gtdb/data/releases/release86/86.1/. The phylogenetic tree was pruned to include only species of interest (see FIG. 16 for the correspondence between species names and respective GTDB representative genomes) using the Ape package (version 5.3) in R (version 3.5.2). The pairwise distances between the tips in the pruned trees were computed using the Ape package 53. The tree was visualized using iTOL (54).

### Protein Sequence Similarity Analysis

To analyze sequence similarities of r-proteins, refseq proteomes of relevant species were downloaded and a local BLAST database was created from these proteomes. E. coli SSU r-protein sequences were queried against the database using local blastp with default parameters using the BLOSUM62 similarity matrix. Hits were filtered to those annotated with “30S,” “SSU,” or “ribosomal protein.”

### Data Availability

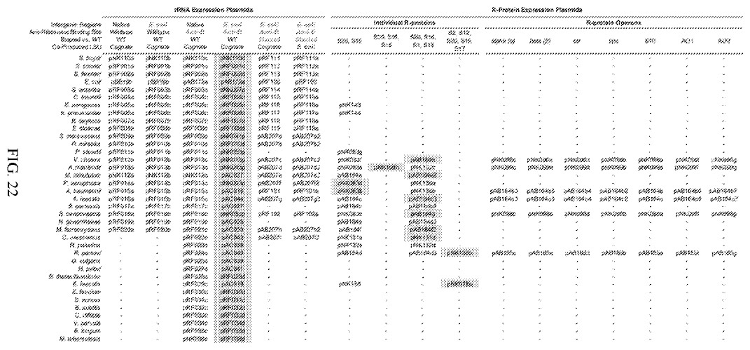

The data that support the findings of this study are available within the paper and its supplementary information files. Plasmid names are provided for reference in FIGS. 21 and 22 below. All plasmids generated in this study are in the process of being deposited in Addgene.

### Example 2: Heterologous rRNA Operons Complemented SQ171 Deficiency

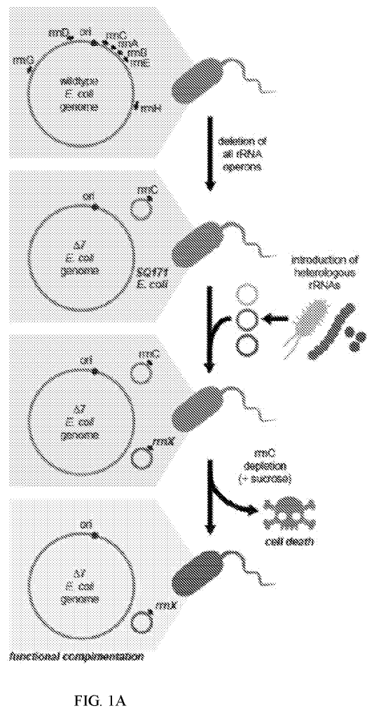

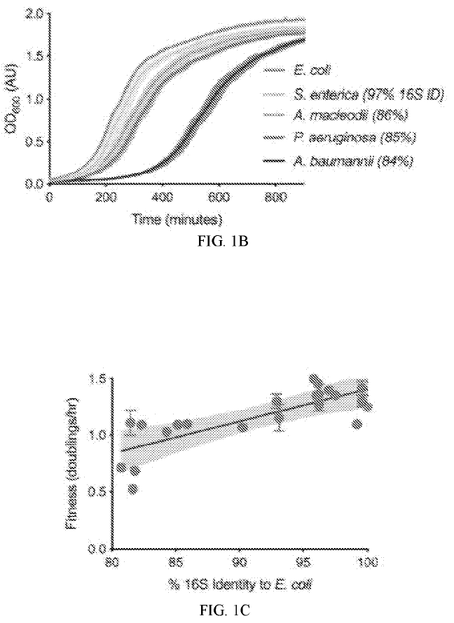

SQ171 is an E. coli strain lacking all seven chromosomal rRNA operons and carrying a single, counter-selectable plasmid bearing the wildtype rrnC operon (17, 30). To investigate the ability of heterologous rRNAs to support SQ171 cell survival, episomally-encoded rRNA operons were introduced into the strain followed by sucrose counterselection of the resident E. coli rrnC plasmid using the B. subtilis sacB cassette (FIG. 1A). Heterologous rRNA operons capable of yielding functional heterologous ribosomes sustained SQ171 growth following sucrose counterselection. Prior work in SQ171 complementation using fully native heterologous rRNA operons was extended to Salmonella typhimurium (96.8% 16S rRNA sequence identity to E. coli) and Proteus vulgaris rRNA (93.2%) (17).

This strategy was validated using a number of increasingly divergent heterologous rRNA operons: Salmonella enterica (97.0% 16S rRNA sequence identity to E. coli), Alteromonas macleodii (85.9%), Pseudomonas aeruginosa (85.2%), and Acinetobacter baumannii (84.3%). Heterologous rRNA derived from S. enterica robustly supported SQ171 strain growth, while rRNA derived from A. macleodii and P. aeruginosa supported growth with a moderate defect (FIG. 1B). Surprisingly, a significant growth defect in SQ171 cells complemented by rRNA derived from A. baumannii was observed despite the minor difference in sequence identity to E. coli rRNA, as compared, e.g., to P. aeruginosa. Motivated by these results, the instant strategy was extended to a total of 21 increasingly divergent rRNA operons from diverse proteobacterial species. Each of these, including an rRNA derived from the zetaproteobacteria Mariprofundus ferrooxydans (80.7%), sustained SQ171 growth (FIG. 14). Consistent with the results obtained using completely native rRNA operons, fused heterologous-E. coli 16S fragments from gammaproteobacterial and betaproteobacterial rRNAs also supported Δ7 strain survival (16). A linear relationship was observed between complemented SQ171 strain fitness and 16S rRNA sequence identity, consistent with prior reports that strains relying on increasingly divergent rRNAs show comparatively reduced fitness (FIG. 1C) (16, 17).

### Example 3: Orthogonal Translation Enabled Quantitative Heterologous Ribosome Assessment

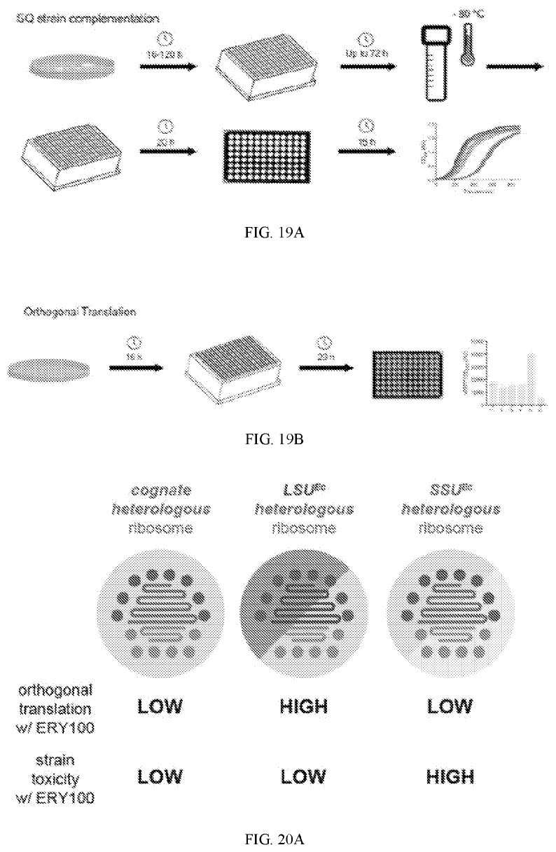

SQ171 complementation provides information about the capacity of a heterologous rRNA to translate the E. coli proteome of >4000 proteins (31), as well as the fulfillment of extracatalytic roles, including integrating environmental cues to modulate translation (29) and initiating the stringent response to cellular stressors (28). Furthermore, hibernation factors that regulate translation in gammaproteobacteria (e.g., E. coli) under unfavorable conditions are often not found in other proteobacterial classes (28), which indicates that phylogenetically distant rRNAs are unable to support SQ171 survival due to regulatory constraints rather than enzymatic ones, further confounding interpretation of strain survival. Finally, it was observed herein that SQ171 complementation pipelines required up to 5 days to observe colonies for strains relying on highly divergent rRNAs, where transformed colonies were laboriously counter-screened due to the high escape frequency of SacB-dependent negative selection (FIG. 19) (32, 33).

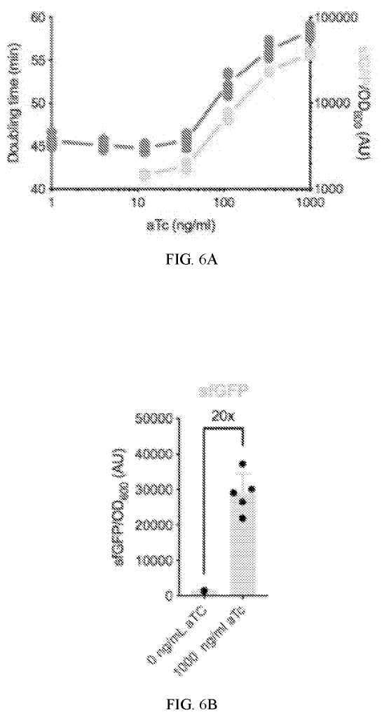

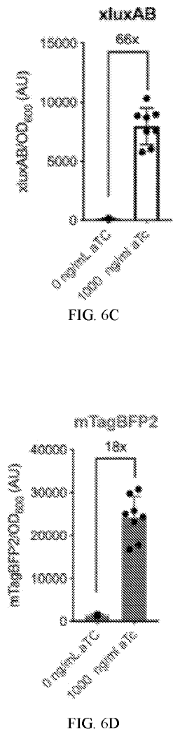

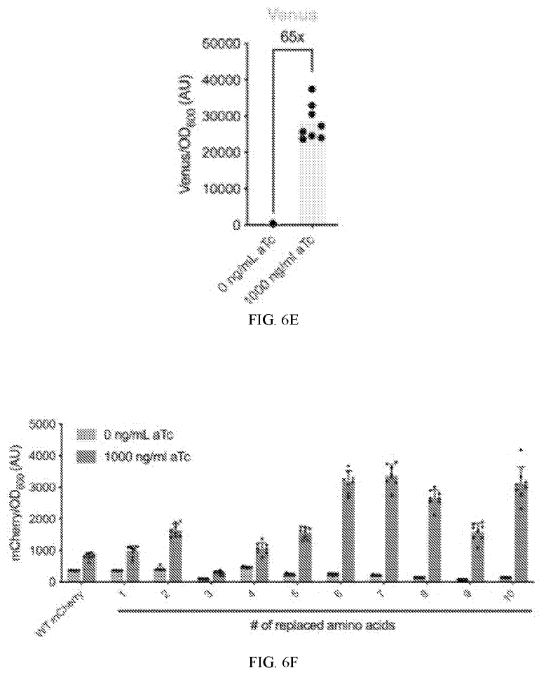

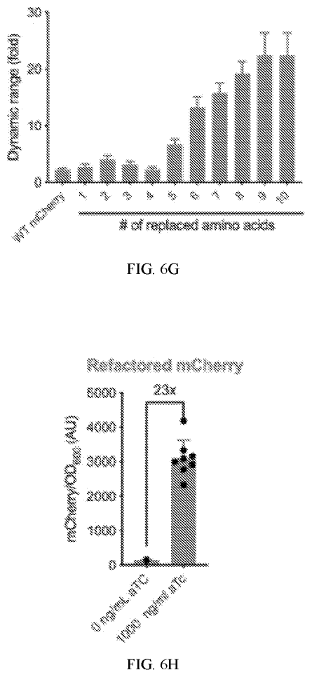

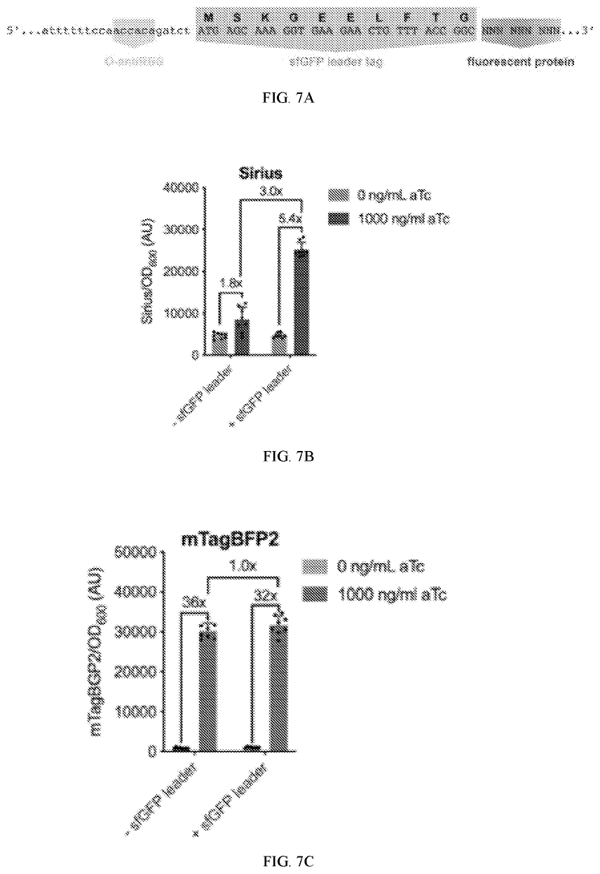

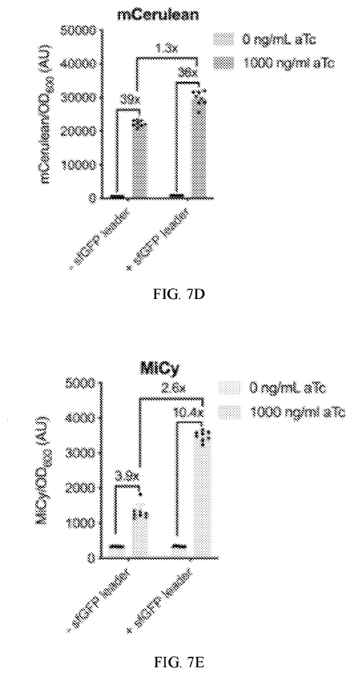

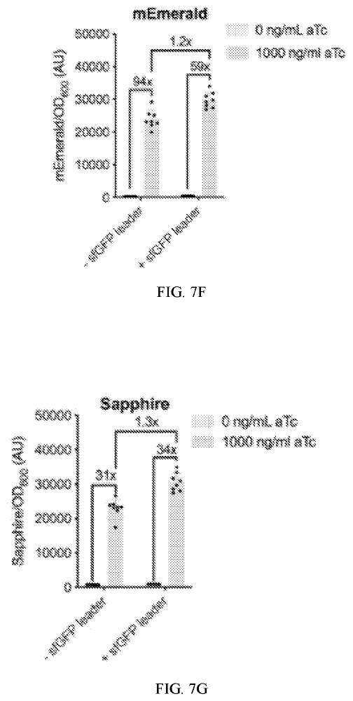

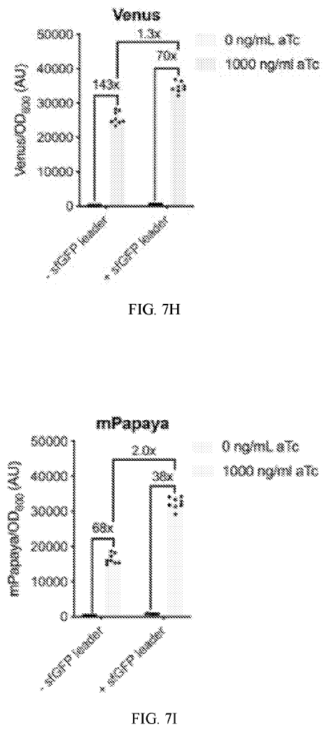

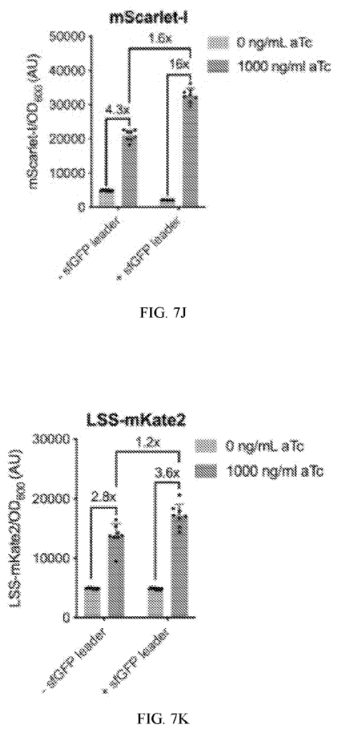

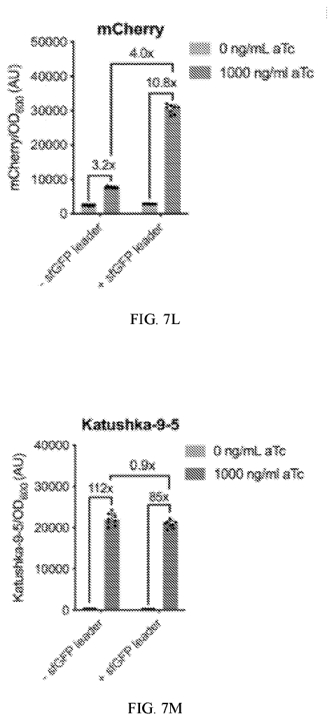

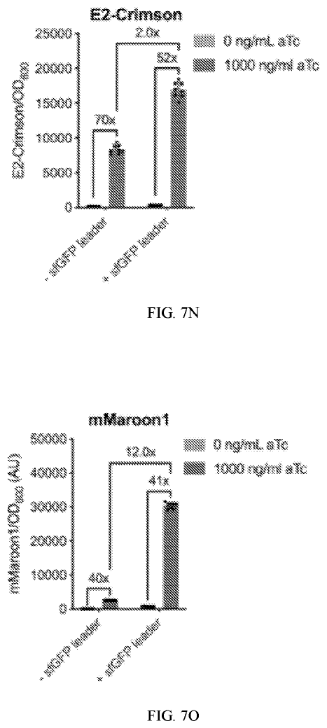

To overcome these technical limitations, an assay was developed herein that delivered a single, quantifiable, translational output orthogonal to native ribosomal machinery and therefore also independent of cell viability. Previously described orthogonal ribosome-mRNA pairs were leveraged, in which the antiRBS of the 16S rRNA was engineered to exclusively translate a researcher-defined transcript bearing a complementary RBS (4, 8, 71-74). This yielded an orthogonal pool of ribosomes (o-ribosomes) in vivo, the functions of which were monitored and quantified via reporter expression (superfolder GFP; sfGFP (34)) independently of cellular survival (FIG. 2A). Importantly, wild-type ribosomes were unable to translate the orthogonal mRNA (o-mRNA) reporter, which ensured that the observed reporter activity was dependent upon engineered o-ribosomes (FIG. 2B). This orthogonal translation genetic circuit did not significantly affect cellular viability, in agreement with prior reports (FIG. 6A) (4, 71-74), and both o-mRNA and o-ribosome production were controlled via small molecule inducers to further limit the cellular burden of their production (FIGS. 2A and 2C). Orthogonal translation was extended to numerous reporter proteins, and subsequently reporter-specific limitations on fluorescent protein functionality were observed (FIGS. 6B to 6H). Accordingly, a ten amino acid sfGFP leader was identified that obviated these constraints and improved orthogonal translation for various reporters (FIGS. 7A to 7P).

With a robust reporter system now in hand, the o-antiRBS was engineered into all 21 heterologous rRNAs capable of complementing SQ171 viability, alongside an additional 13 phylogenetically more divergent rRNAs (FIG. 14). The activity of all 34 o-rRNAs were quantified via orthogonal translation. It was thereby discovered that most rRNAs capable of supporting SQ171 growth similarly synthesized sfGFP at robust levels (FIG. 2D), with the exception of o-rRNAs derived from Serratia marcescens (96.0% 16S rRNA sequence identity to E. coli), Vibrio cholerae (90.3%), P. aeruginosa (85.2%), A. baumannii (84.3%), Alcaligenes faecalis (82.3%), Bordetella pertussis (81.6%), Burkholderia cenocepacia (81.5%), and M. ferrooxydans (80.7%). Notably, sfGFP translation fell markedly with phylogenetic distance from E. coli, wherein heterologous rRNAs exclusively derived from gammaproteobacteria and betaproteobacteria were capable of translating sfGFP (FIG. 2D). Supporting this observation, a robust correlation between 16S rRNA sequence identity to E. coli and orthogonal translation activity was observed (FIG. 2E). A robust correlation between complemented SQ171 fitness and orthogonal translation activity for each functional heterologous rRNA (FIG. 2F) was also observed. Collectively, these findings supported the use of orthogonal translation (as disclosed herein) in lieu of SQ171 complementation to quantify the translational activity of heterologous ribosomes.

### Example 4: Engineered rRNA Processing Improved Heterologous Ribosome Activity

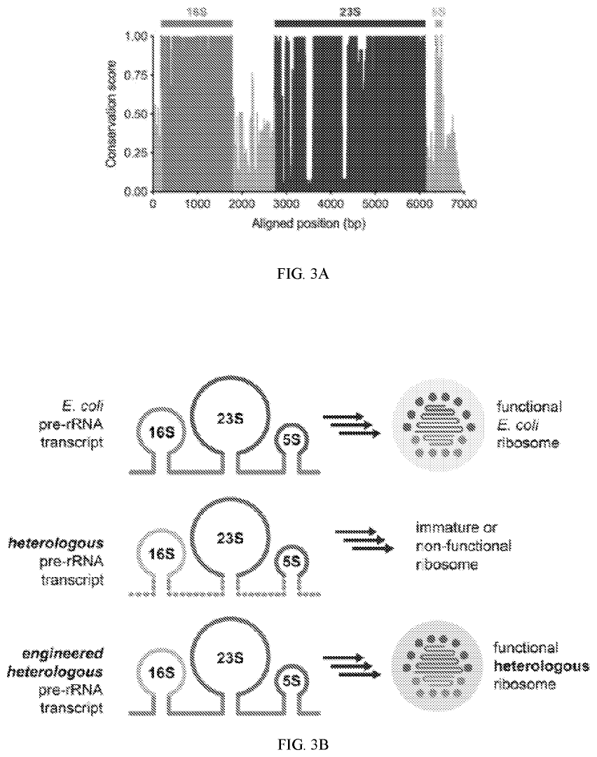

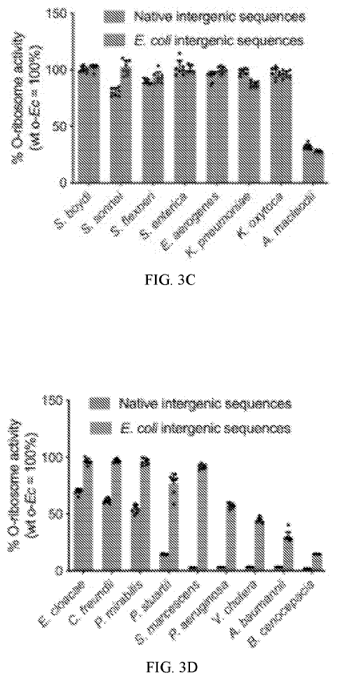

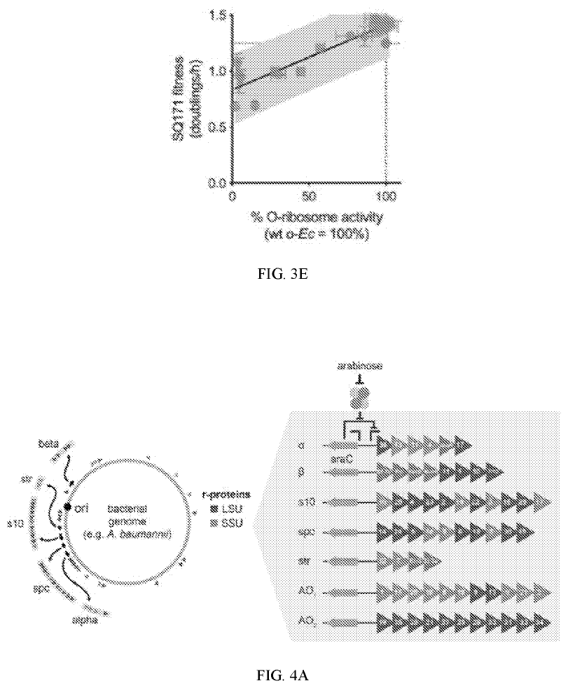

The observed relationship between heterologous orthogonal translation activity and phylogenetic distance from E. coli indicated that certain elements encoded within the rRNA operon might have sufficiently diverged to restrict efficient ribosome assembly in E. coli. Analysis of per-base conservation (35) across the complete rRNA operons showed significantly higher conservation scores within the ribosomal genes (16S, 23S, and 5S rRNAs), as compared to intergenic elements (FIG. 3A). Intergenic sequences flanking each rRNA gene have been described as crucial to ribosome biogenesis, as they direct pre-rRNA transcript folding and processing by RNAses (3, 36, 37). It was hypothesized that E. coli RNases would fail to recognize divergent sequences on non-native rRNA transcripts, yielding immature or poorly processed heterologous ribosomes (FIG. 3B). Herein, it was discovered that substitution of these elements with their E. coli counterparts robustly corrected the rRNA processing defect and improved overall orthogonal translation activity.

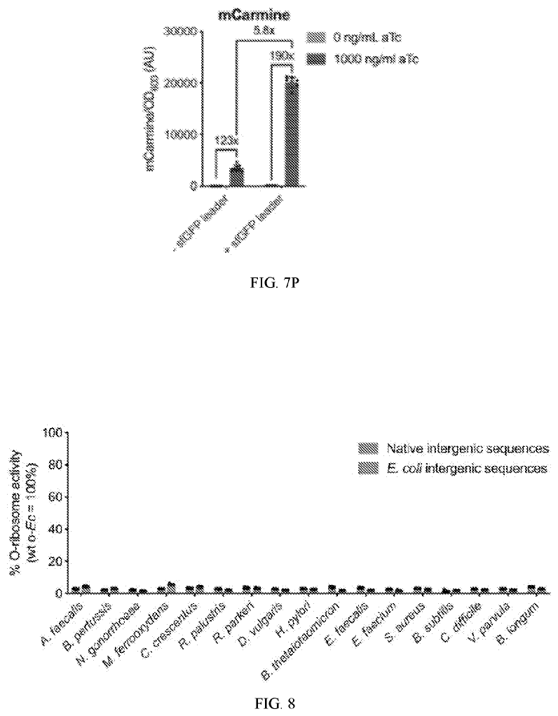

To assess the impact of putative rRNA processing on heterologous ribosome function in E. coli, the native intergenic sequences of each of the 34 o-rRNAs were substituted with their corresponding E. coli sequences (FIG. 3B). Substitution of intergenic sequences for o-rRNAs with high 16S identity to E. coli (96.2-99.6%) exerted a minimal effect on sfGFP expression (FIG. 3C). However, replacement of intergenic sequences for moderately divergent o-rRNAs (81.5-96.2%) significantly increased sfGFP expression (FIG. 3D). Notably, many nonfunctional o-rRNAs yielded robust sfGFP activities only after intergenic sequence replacement, namely S. marcescens, V. cholerae, P. aeruginosa, A. baumannii, and B. cenocepacia. Replacement of intergenic sequences for highly divergent o-rRNAs (69.8-82.3%) failed to improve o-rRNA translation (FIG. 8), indicating that further engineering or supplementation with additional factors was necessary for improving the activity of these highly divergent heterologous ribosomes. Finally, the wildtype antiRBS was introduced into the 21 engineered intergenic sequence-bearing rRNAs for which the native counterparts were previously found to support SQ171 survival. Notably, it was found that SQ171 survival was maintained after intergenic sequence replacement, as was the relationship between SQ171 fitness and orthogonal translation activity (FIG. 3E). Taken together, these data indicated that rRNA processing limited the assembly of more divergent heterologous rRNAs into functional ribosomes, and that engineering processing sites significantly improved the activities of refractory heterologous ribosomes.

### Example 5: R-Protein Complementation Enhanced Heterologous Ribosome Activity

Highly divergent rRNAs (<80% 16S rRNA sequence identity to E. coli) failed to translate the orthogonal sfGFP transcript despite replacement of their intergenic sequences, indicating that the formation of functional heterologous ribosomes required supplementation with additional factors. As ribosomal proteins (r-proteins) are known to co-diverge alongside their cognate rRNA (38, 39, 40), it was hypothesized herein that E. coli r-proteins might only be capable of binding to and forming heterologous ribosomes with rRNAs sufficiently homologous to E. coli. Complementing highly divergent rRNAs with cognate r-proteins might therefore be expected to improve heterologous ribosome activity.

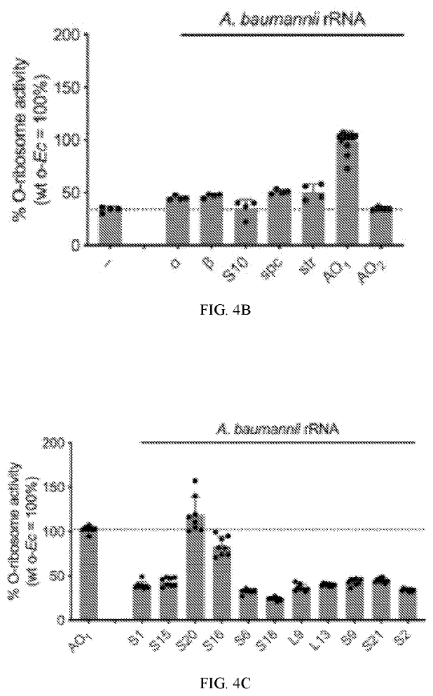

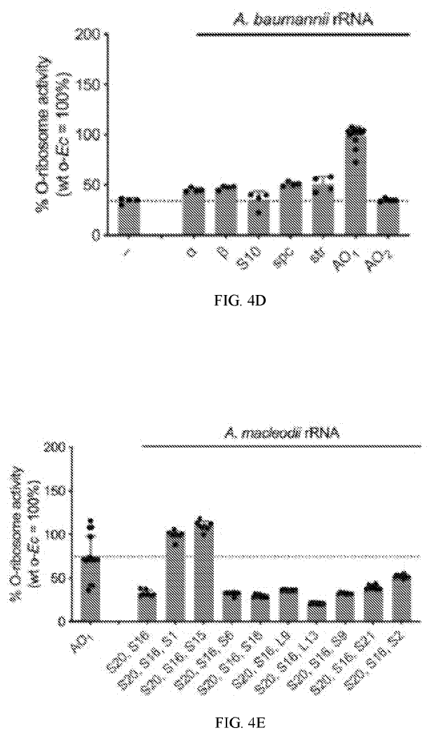

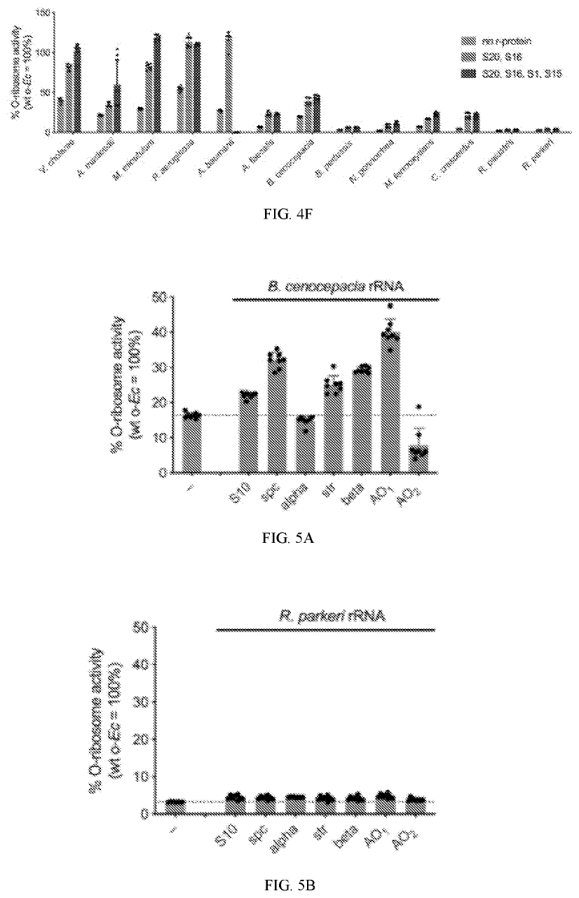

In prokaryotes, the majority of r-proteins are typically arranged on five operons (α, β, s10, spc, str). R-proteins encoded within these five operons account for ˜60% (12/21 SSU and 18/33 LSU in E. coli) of the full r-protein repertoire (31) with the remaining ˜40% distributed throughout the genome (FIG. 4A). Using A. baumannii o-rRNA bearing the E. coli intergenic sequences (30% activity vs. E. coli o-rRNA), potential improvements in activity were analyzed when expressing the full set of 55 cognate r-proteins distributed through seven plasmids: five corresponding to the naturally occurring r-protein operons and two artificial operons (AOs) encoding the remaining r-proteins (FIG. 4A). To capture potential epistatic interactions involving either SSU or LSU r-proteins, each artificial operon was enriched in either SSU (AO1) or LSU (AO2) r-proteins.

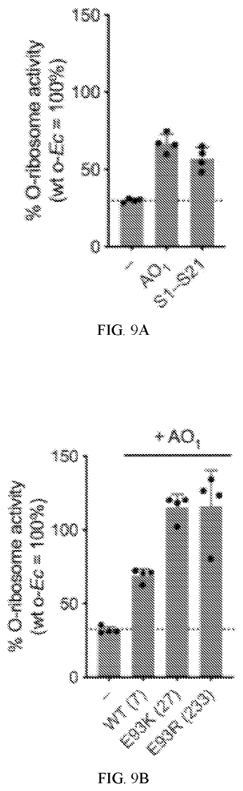

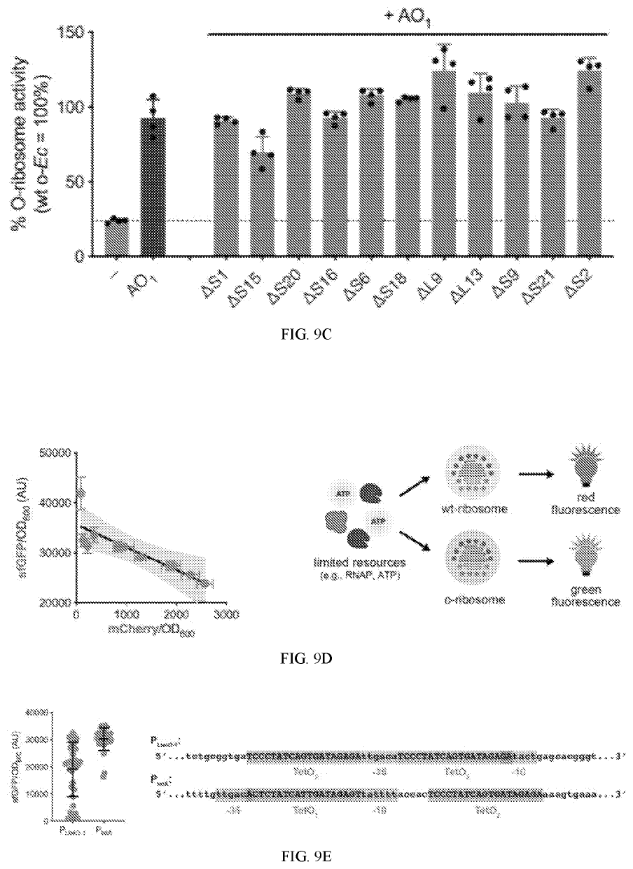

When tested alongside A. baumannii o-rRNA, only AbAO1 (comprising mostly SSU r-proteins) significantly improved sfGFP expression (FIG. 4B). Notably, complementation by a plasmid containing every A. baumannii SSU r-protein (S1-521) yielded similar levels of activity as AbAO1, which indicated that the latter contains all SSU r-proteins necessary to improve A. baumannii heterologous translation (FIG. 9A). Copy-up mutations (41) to AbAO1 further improved observed activity of this heterologous ribosome, exceeding the activity level of the E. coli o-rRNA (FIG. 9B). To identify specific r-proteins responsible for this increase in heterologous ribosome activity, r-proteins were sequentially deleted from AbAO1. Remarkably, it was identified that robust sfGFP activity was maintained in all instances (FIG. 9C), indicating that one or more r-proteins were functionally redundant. Analysis of individual r-proteins confirmed this assessment, highlighting that expression of either AbS20 or AbS16 improved A. baumannii heterologous o-ribosome activity to levels comparable to the E. coli o-rRNA (FIG. 4C).

It is notable that excessive protein overexpression alongside orthogonal translation circuits has pleiotropic consequences on apparent translational activity. Using mCherry as a surrogate for cognate r-protein overexpression alongside o-rRNA-dependent sfGFP production, a characteristic isocost line was observed that described the production of two proteins under the constraints of a restricted metabolic budget (FIG. 9D) (42). Furthermore, o-rRNA promoter choice dramatically affected orthogonal translation activity, as promoters with repetitive elements were rapidly recombined under high expression to mitigate the associated ribosome production burden (FIG. 9E).

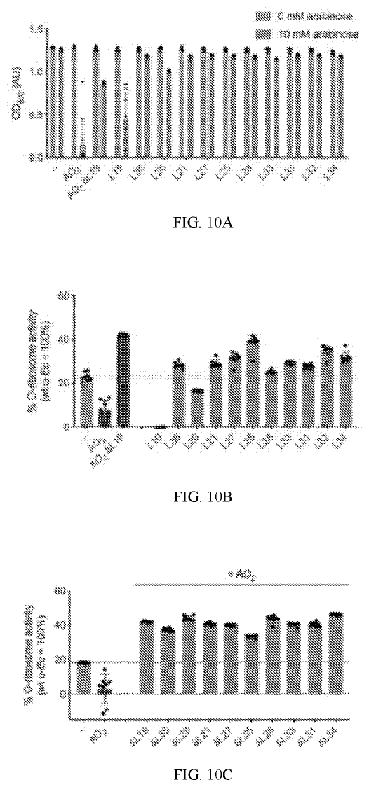

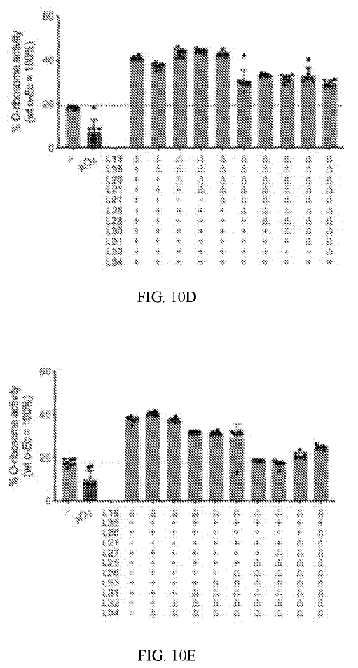

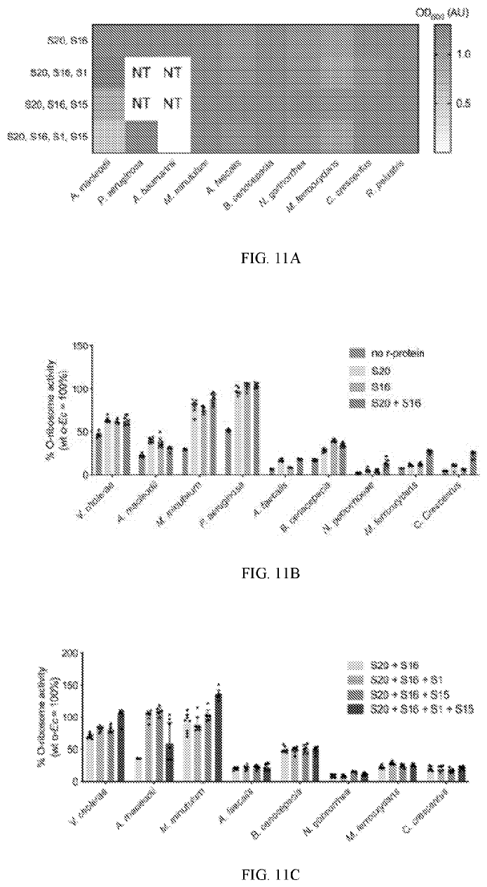

This analysis was extended to A. macleodii o-rRNA bearing the E. coli intergenic sequences (17% activity vs. E. coli o-rRNA). Again it was found that only AmAO1 significantly improved sfGFP expression (FIG. 4D). As this finding indicated an overlap with A. baumannii r-proteins that improved heterologous ribosome function, AmAO1 constituent proteins were expressed alongside AmS20 and AmS16. It was found that combinations of either AmS20+AmS16+AmS1 or AmS20+AmS16+AmS15 were sufficient to improve A. macleodii o-rRNA function to levels comparable with the E. coli o-rRNA (FIG. 4E). A smaller but significant increase in apparent orthogonal translation activity was observed using AmAO2 (enriched in LSU r-proteins) (FIGS. 4D, 10A and 10B). However, AmAO2 r-proteins did not provide a comparable set of complementing r-proteins, as most genes contributed minor enhancements that collectively improved orthogonal translation activity (FIGS. 10B to 10E). These results confirmed that complementation with only a small number of cognate r-proteins exhibited significant effects on heterologous ribosome function in E. coli.

### Example 6: rRNA Divergence Predicted Rules for Cognate R-protein Complementation

The interface between rRNA and r-proteins is subject to extensive coevolution and divergence between related organisms (38-40). Accordingly, overlap between SSU r-protein complements that improved A. baumannii and A. macleodii o-rRNA activity indicated that the same r-proteins might improve the function of o-rRNAs derived from a variety of species. Indeed, the identified r-protein combinations improved activities of increasingly distant o-rRNAs: P. aeruginosa, V. cholerae, Marinospirillum minutulum, A. faecalis, B. cenocepacia, Neisseria gonorrhoeae, M. ferrooxydans, and Caulobacter crescentus (FIG. 4F).

Notably, this complete set of four r-proteins (S20, S16, S1 and S15) was not necessary for the observed improvement in activity for all evaluated o-rRNAs (FIGS. 11B and 11C). S20 and S16 are functionally redundant when expressed alongside cognate o-rRNAs derived from species more phylogenetically related to E. coli: V. cholerae, A. macleodii, M. minutulum, P. aeruginosa, and B. cenocepacia. Uniquely, S16 exhibited no effect on A. faecalis o-translation, where only S20 improved apparent activity. However, both proteins were necessary for enhanced activity when expressed alongside o-rRNAs derived from the more distant species N. gonorrheae, M. ferrooxydans, and C. crescentus (FIG. 11B). Extending the analysis to the complete set of four proteins, it was found that the addition of both S1 and S15 was necessary for maximal activities of V. cholerae and M. minutulum o-rRNAs, but neither r-protein had an effect when expressed alongside S20 and S16 for o-rRNAs derived from more divergent species (FIG. 8C).

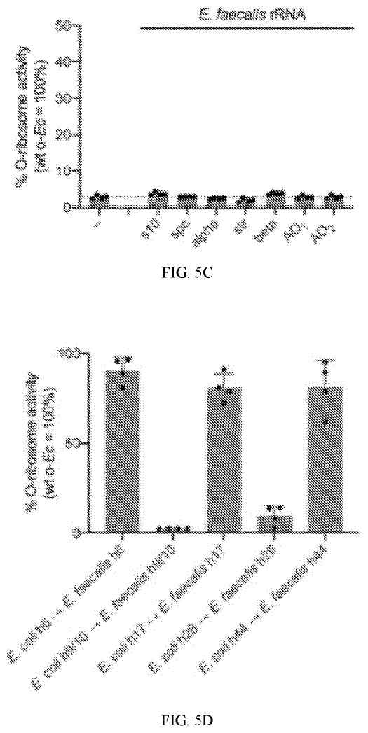

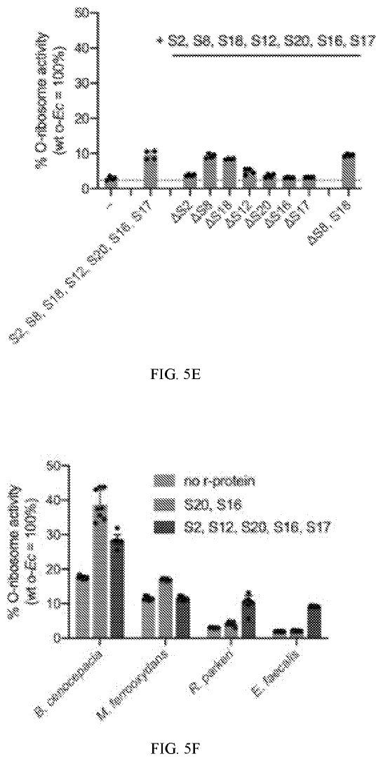

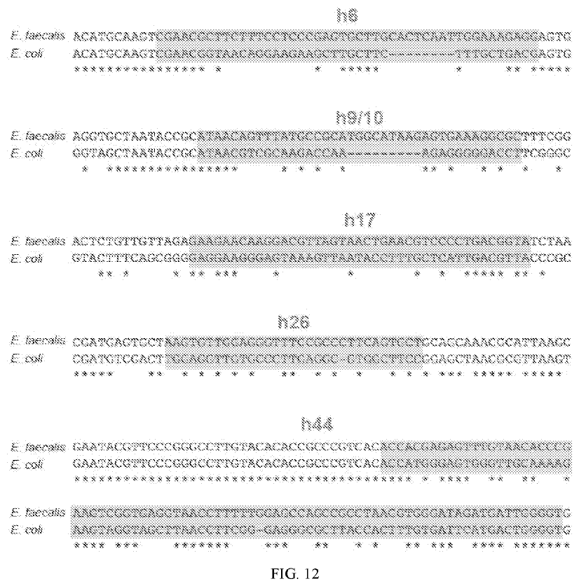

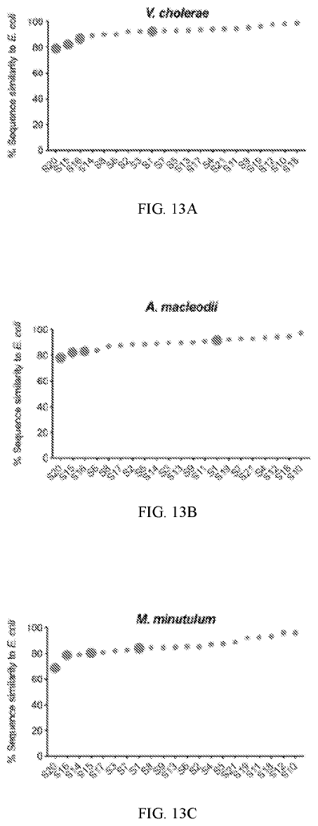

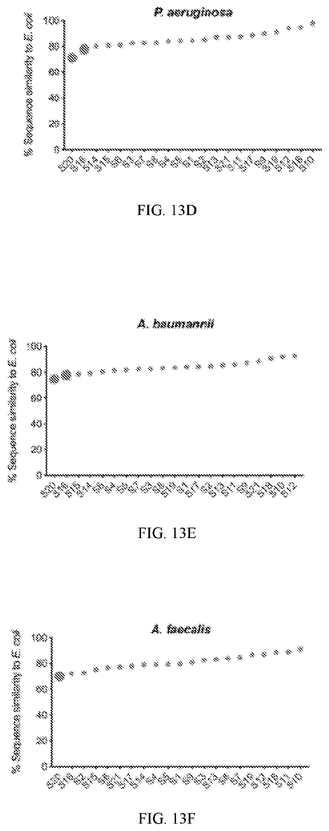

To determine r-proteins necessary to complement more divergent o-rRNAs, the instantly disclosed operon-based complementation approach was extended to rRNAs derived from B. cenocepacia (betaproteobacteria; 81.5% 16S rRNA sequence ID to E. coli), Rickettsia parkeri (alphaproteobacteria; 76.8%), and Enterococcus faecalis (bacilli; 76.1%). However, a significant increase in orthogonal translation activity upon r-protein operon induction (FIGS. 5A to 5C) was not observed. Having found sequence divergence from E. coli to be a powerful predictor of relevant features for heterologous rRNA supplementation, 5 regions were then manually identified in the E. faecalis 16S rRNA possessing particularly low sequence identity to E. coli, via pairwise alignment (FIG. 12) (43). As these divergent elements make extensive contacts with r-proteins in the E. coli ribosome (PDB: 4YBB) (44), significant divergence from E. coli in these sequences indicated an inability to efficiently bind to the requisite r-proteins.

To validate the functional relevance of these helices, variants of the E. coli o-rRNA were constructed in which these helices were replaced with their cognate E. faecalis helices, with the discovery that orthogonal translation was abrogated in only 2 instances (transplantation of helices h9/h10 and h26, FIG. 5D). Seven r-proteins were expected to bind these helices based on existing ribosomal structures (EfS2, EfS8, EfS18, EfS12, EfS20, EfS16, and EfS17) (44), and indeed supplementation with this set yielded a detectable increase in orthogonal translation activity (FIG. 5E). The deletion of EfS8 and EfS18 from this set of proteins exerted no effect on activity, resulting in a set of 5 proteins that allowed E. faecalis o-rRNA activity to reach levels equivalent to 9.5% of the E. coli o-rRNA (FIG. 5E). Notably, this same set of 5 r-proteins was less effective than the combination of S20 and S16 for B. cenocepacia and M. ferrooxydans (81.5% and 80.1% 16S rRNA sequence identity to E. coli, respectively), but was more effective for the more distantly related R. parkeri (76.8%) and E. faecalis (76.1%) (FIG. 5F). Without wishing to be bound by theory, for o-rRNAs derived from more divergent organisms, the complete set of 5 r-proteins might be necessary to form a functional complex that cannot be formed by E. coli r-proteins. At the same time, for o-rRNAs more related to E. coli, cognate r-proteins might compete with E. coli proteins for binding, forming less functional ribosomes. This finding highlighted the importance of the instant disclosure's identification of the minimal subset of r-proteins necessary to improve function. Furthermore, it was noted that this set of 5 r-proteins was distributed across 2 naturally occurring operons in addition to the artificial operon AO1, obscuring these interactions from the 7-operon approach used above. Collectively, these results indicated that rRNA/r-protein codivergence was useful in predicting r-protein repertoires that enhanced the activity of heterologous ribosomes in E. coli.

### Example 7: Assessment of the Exchange Between E. coli and Heterologous Ribosome Subunits

The analysis of the instant disclosure provided guidelines to improve refractory heterologous ribosome function; however, exclusive identification of SSU r-proteins suggested that cognate heterologous LSUs were poorly active in E. coli. It was hypothesized that either E. coli LSUs interacted with heterologous SSUs to enable orthogonal translation, or, that because many heterologous rRNAs supported SQ171 strain survival, cognate heterologous LSUs were sufficiently active (FIGS. 1C, 2F, 3E and 14).

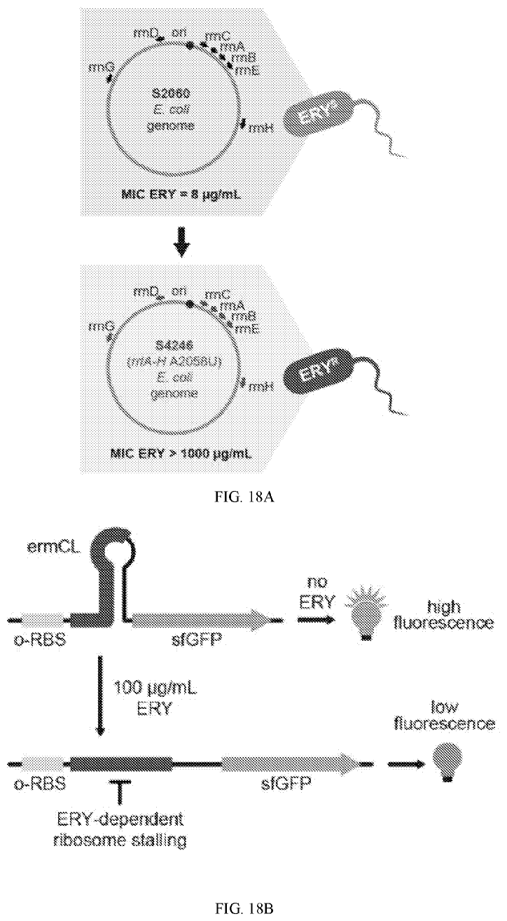

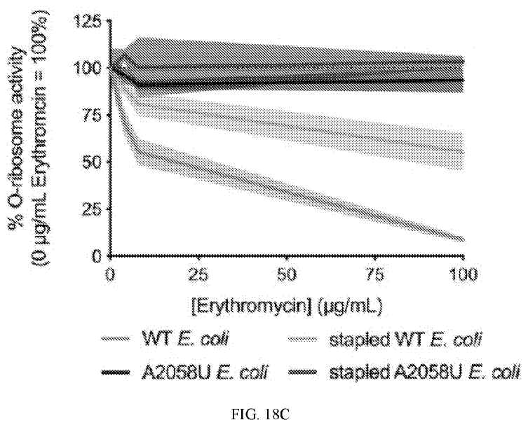

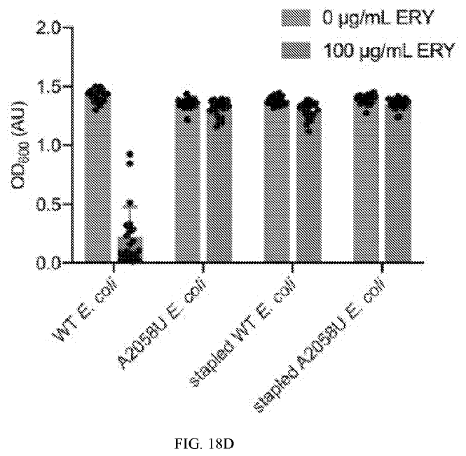

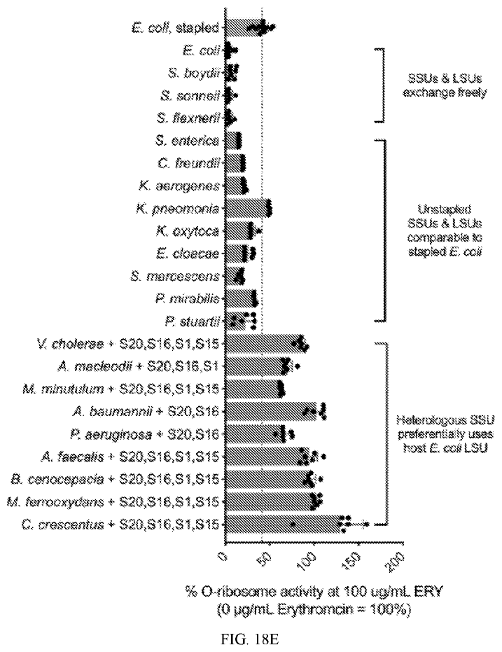

To assess the degree of association between E. coli LSUs and heterologous SSUs, an erythromycin-dependent reporter was developed to distinguish between genome- (E. coli; erythromycin-resistant) and episome-derived (heterologous; erythromycin-sensitive) LSUs. The erythromycin-resistant strain S4246 was developed, wherein all seven genomic 23S genes (rrlA-H) of 52060 cells were mutated (A2058U) (75) to mitigate macrolide binding in the ribosomal exit tunnel (FIG. 18A) (76, 77). rrlA-H A2058U sequences are presented in Table 1.

TABLE 1
rrlA-H A2058U Sequences

| Name | Sequence |
|---|---|
| rrlA | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGCGAACGGGGAGCAGCCCAGAGCCTGAATCAGTGTGTG |
| | TGTTAGTGGAAGCGTCTGGAAAGGCGTGCGATACAGGGTGACAGCCCC |
| | GTACACAAAAATGCACATGCTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCACGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAATTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACACTATGTGTTGTTGGGTAGGGGAGCGTTCTGTAAGC |
| | CTGTGAAGGTGTGCTGTGAGGCATGCTGGAGGTATCAGAAGTGCGAAT |
| | GCTGACATAAGTAACGATAAAGCGGGTGAAAAGCCCGCTCGCCGGAAG |
| | ACCAAGGGTTCCTGTCCAACGTTAATCGGGGCAGGGTGAGTCGACCCCT |
| | AAGGCGAGGCCGAAAGGCGTAGTCGATGGGAAACAGGTTAATATTCCT |
| | GTACTTGGTGTTACTGCGAAGGGGGGACGGAGAAGGCTATGTTGGCCG |
| | GGCGACGGTTGTCCCGGTTTAAGCGTGTAGGCTGGTTTTCCAGGCAAAT |
| | CCGGAAAATCAAGGCTGAGGCGTGATGACGAGGCACTACGGTGCTGAA |
| | GCAACAAATGCCCTGCTTCCAGGAAAAGCCTCTAAGCATCAGGTAACA |
| | TCAAATCGTACCCCAAACCGACACAGGTGGTCAGGTAGAGAATACCAA |
| | GGCGCTTGAGAGAACTCGGGTGAAGGAACTAGGCAAAATGGTGCCGTA |
| | ACTTCGGGAGAAGGCACGCTGATATGTAGGTGAAGCGACTTGCTCGTG |
| | GAGCTGAAATCAGTCGAAGATACCAGCTGGCTGCAACTGTTTATTAAAA |
| | ACACAGCACTGTGCAAACACGAAAGTGGACGTATACGGTGTGACGCCT |
| | GCCCGGTGCCGGAAGGTTAATTGATGGGGTTAGCCGCAAGGCGAAGCT |
| | CTTGATCGAAGCCCCGGTAAACGGCGGCCGTAACTATAACGGTCCTAA |
| | GGTAGCGAAATTCCTTGTCGGGTAAGTTCCGACCTGCACGAATGGCGTA |
| | ATGATGGCCAGGCTGTCTCCACCCGAGACTCAGTGAAATTGAACTCGCT |
| | GTGAAGATGCAGTGTACCCGCGGCAAGACGGTAAGACCCCGTGAACCT |
| | TTACTATAGCTTGACACTGAACATTGAGCCTTGATGTGTAGGATAGGTG |
| | GGAGGCTTTGAAGTGTGGACGCCAGTCTGCATGGAGCCGACCTTGAAA |
| | TACCACCCTTTAATGTTTGATGTTCTAACGTTGACCCGTAATCCGGGTTG |
| | CGGACAGTGTCTGGTGGGTAGTTTGACTGGGGCGGTCTCCTCCTAAAGA |
| | GTAACGGAGGAGCACGAAGGTTGGCTAATCCTGGTCGGACATCAGGAG |
| | GTTAGTGCAATGGCATAAGCCAGCTTGACTGCGAGCGTGACGGCGCGA |
| | GCAGGTGCGAAAGCAGGTCATAGTGATCCGGTGGTTCTGAATGGAAGG |
| | GCCATCGCTCAACGGATAAAAGGTACTCCGGGGATAACAGGCTGATAC |
| | CGCCCAAGAGTTCATATCGACGGCGGTGTTTGGCACCTCGATGTCGGCT |
| | CATCACATCCTGGGGCTGAAGTAGGTCCCAAGGGTATGGCTGTTCGCCA |
| | TTTAAAGTGGTACGCGAGCTGGGTTTAGAACGTCGTGAGACAGTTCGGT |
| | CCCTATCTGCCGTGGGCGCTGGAGAACTGAGGGGGGCTGCTCCTAGTAC |
| | GAGAGGACCGGAGTGGACGCATCACTGGTGTTCGGGTTGTCATGCCAA |
| | TGGCACTGCCCGGTAGCTAAATGCGGAAGAGATAAGTGCTGAAAGCAT |
| | CTAAGCACGAAACTTGCCCCGAGATGAGTTCTCCCTGACTCCTTGAGAG |
| | TCCTGAAGGAACGTTGAAGACGACGACGTTGATAGGCCGGGTGTGTAA |
| | GCGCAGCGATGCGTTGAGCTAACCGGTACTAATGAACCGTGAGGCTTA |
| | ACCTT (SEQ ID NO: 388) |
| rrlB | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGCGAACGGGGAGCAGCCCAGAGCCTGAATCAGTGTGTG |
| | TGTTAGTGGAAGCGTCTGGAAAGGCGCGCGATACAGGGTGACAGCCCC |
| | GTACACAAAAATGCACATGCTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCACGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAATTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACGCTTATGCGTTGTTGGGTAGGGGAGCGTTCTGTAAG |
| | CCTGCGAAGGTGTGCTGTGAGGCATGCTGGAGGTATCAGAAGTGCGAA |
| | TGCTGACATAAGTAACGATAAAGCGGGTGAAAAGCCCGCTCGCCGGAA |
| | GACCAAGGGTTCCTGTCCAACGTTAATCGGGGCAGGGTGAGTCGACCC |
| | CTAAGGCGAGGCCGAAAGGCGTAGTCGATGGGAAACAGGTTAATATTC |
| | CTGTACTTGGTGTTACTGCGAAGGGGGGACGGAGAAGGCTATGTTGGC |
| | CGGGCGACGGTTGTCCCGGTTTAAGCGTGTAGGCTGGTTTTCCAGGCAA |
| | ATCCGGAAAATCAAGGCTGAGGCGTGATGACGAGGCACTACGGTGCTG |
| | AAGCAACAAATGCCCTGCTTCCAGGAAAAGCCTCTAAGCATCAGGTAA |
| | CATCAAATCGTACCCCAAACCGACACAGGTGGTCAGGTAGAGAATACC |
| | AAGGCGCTTGAGAGAACTCGGGTGAAGGAACTAGGCAAAATGGTGCCG |
| | TAACTTCGGGAGAAGGCACGCTGATATGTAGGTGAGGTCCCTCGCGGA |
| | TGGAGCTGAAATCAGTCGAAGATACCAGCTGGCTGCAACTGTTTATTAA |
| | AAACACAGCACTGTGCAAACACGAAAGTGGACGTATACGGTGTGACGC |
| | CTGCCCGGTGCCGGAAGGTTAATTGATGGGGTTAGCGCAAGCGAAGCT |
| | CTTGATCGAAGCCCCGGTAAACGGCGGCCGTAACTATAACGGTCCTAA |
| | GGTAGCGAAATTCCTTGTCGGGTAAGTTCCGACCTGCACGAATGGCGTA |
| | ATGATGGCCAGGCTGTCTCCACCCGAGACTCAGTGAAATTGAACTCGCT |
| | GTGAAGATGCAGTGTACCCGCGGCAAGACGGTAAGACCCCGTGAACCT |
| | TTACTATAGCTTGACACTGAACATTGAGCCTTGATGTGTAGGATAGGTG |
| | GGAGGCTTTGAAGTGTGGACGCCAGTCTGCATGGAGCCGACCTTGAAA |
| | TACCACCCTTTAATGTTTGATGTTCTAACGTTGACCCGTAATCCGGGTTG |
| | CGGACAGTGTCTGGTGGGTAGTTTGACTGGGGCGGTCTCCTCCTAAAGA |
| | GTAACGGAGGAGCACGAAGGTTGGCTAATCCTGGTCGGACATCAGGAG |
| | GTTAGTGCAATGGCATAAGCCAGCTTGACTGCGAGCGTGACGGCGCGA |
| | GCAGGTGCGAAAGCAGGTCATAGTGATCCGGTGGTTCTGAATGGAAGG |
| | GCCATCGCTCAACGGATAAAAGGTACTCCGGGGATAACAGGCTGATAC |
| | CGCCCAAGAGTTCATATCGACGGCGGTGTTTGGCACCTCGATGTCGGCT |
| | CATCACATCCTGGGGCTGAAGTAGGTCCCAAGGGTATGGCTGTTCGCCA |
| | TTTAAAGTGGTACGCGAGCTGGGTTTAGAACGTCGTGAGACAGTTCGGT |
| | CCCTATCTGCCGTGGGCGCTGGAGAACTGAGGGGGGCTGCTCCTAGTAC |
| | GAGAGGACCGGAGTGGACGCATCACTGGTGTTCGGGTTGTCATGCCAA |
| | TGGCACTGCCCGGTAGCTAAATGCGGAAGAGATAAGTGCTGAAAGCAT |
| | CTAAGCACGAAACTTGCCCCGAGATGAGTTCTCCCTGACCCTTTAAGGG |
| | TCCTGAAGGAACGTTGAAGACGACGACGTTGATAGGCCGGGTGTGTAA |
| | GCGCAGCGATGCGTTGAGCTAACCGGTACTAATGAACCGTGAGGCTTA |
| | ACCTT (SEQ ID NO: 390) |
| rrlC | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGCGAACGGGGAGCAGCCCAGAGCCTGAATCAGTATGTG |
| | TGTTAGTGGAAGCGTCTGGAAAGGCGCGCGATACAGGGTGACAGCCCC |
| | GTACACAAAAATGCACATATTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCACGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAATTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACGCTTATGCGTTGTTGGGTAGGGGAGCGTTCTGTAAG |
| | CCTGCGAAGGTGTGCTGTGAGGCATGCTGGAGGTATCAGAAGTGCGAA |
| | TGCTGACATAAGTAACGATAAAGCGGGTGAAAAGCCCGCTCGCCGGAA |
| | GACCAAGGGTTCCTGTCCAACGTTAATCGGGGCAGGGTGAGTCGACCC |
| | CTAAGGCGAGGCCGAAAGGCGTAGTCGATGGGAAACAGGTTAATATTC |
| | CTGTACTTGGTGTTACTGCGAAGGGGGGACGGAGAAGGCTATGTTGGC |
| | CGGGCGACGGTTGTCCCGGTTTAAGCGTGTAGGCTGGTTTTCCAGGCAA |
| | ATCCGGAAAATCAAGGCTGAGGCGTGATGACGAGGCACTACGGTGCTG |
| | AAGCAACAAATGCCCTGCTTCCAGGAAAAGCCTCTAAGCATCAGGTAA |
| | CATCAAATCGTACCCCAAACCGACACAGGTGGTCAGGTAGAGAATACC |
| | AAGGCGCTTGAGAGAACTCGGGTGAAGGAACTAGGCAAAATGGTGCCG |
| | TAACTTCGGGAGAAGGCACGCTGATATGTAGGTGAAGCGACTTGCTCGT |
| | GGAGCTGAAATCAGTCGAAGATACCAGCTGGCTGCAACTGTTTATTAAA |
| | AACACAGCACTGTGCAAACACGAAAGTGGACGTATACGGTGTGACGCC |
| | TGCCCGGTGCCGGAAGGTTAATTGATGGGGTCAGCGCAAGCGAAGCTC |
| | TTGATCGAAGCCCCGGTAAACGGCGGCCGTAACTATAACGGTCCTAAG |
| | GTAGCGAAATTCCTTGTCGGGTAAGTTCCGACCTGCACGAATGGCGTAA |
| | TGATGGCCAGGCTGTCTCCACCCGAGACTCAGTGAAATTGAACTCGCTG |
| | TGAAGATGCAGTGTACCCGCGGCAAGACGGTAAGACCCCGTGAACCTT |
| | TACTATAGCTTGACACTGAACATTGAGCCTTGATGTGTAGGATAGGTGG |
| | GAGGCTTTGAAGTGTGGACGCCAGTCTGCATGGAGCCGACCTTGAAAT |
| | ACCACCCTTTAATGTTTGATGTTCTAACGTGGACCCGTGATCCGGGTTG |
| | CGGACAGTGTCTGGTGGGTAGTTTGACTGGGGCGGTCTCCTCCTAAAGA |
| | GTAACGGAGGAGCACGAAGGTTGGCTAATCCTGGTCGGACATCAGGAG |
| | GTTAGTGCAATGGCATAAGCCAGCTTGACTGCGAGCGTGACGGCGCGA |
| | GCAGGTGCGAAAGCAGGTCATAGTGATCCGGTGGTTCTGAATGGAAGG |
| | GCCATCGCTCAACGATAAAAGGTACTCCGGGGATAACAGGCTGATAC |
| | CGCCCAAGAGTTCATATCGACGGCGGTGTTTGGCACCTCGATGTCGGCT |
| | CATCACATCCTGGAGCTGAAGTAGGTCCCAAGGGTATGGCTGTTCGCCA |
| | TTTAAAGTGGTACGCGAGCTGGGTTTAGAACGTCGTGAGACAGTTCGGT |
| | CCCTATCTGCCGTGGGCGCTGGAGAACTGAGGGGGGCTGCTCCTAGTAC |
| | GAGAGGACCGGAGTGGACGCATCACTGGTGTTCGGGTTGTCATGCCAA |
| | TGGCACTGCCCGGTAGCTAAATGCGGAAGAGATAAGTGCTGAAAGCAT |
| | CTAAGCACGAAACTTGCCCCGAGATGAGTTCTCCCTGACTCCTTGAGAG |
| | TCCTGAAGGAACGTTGAAGACGACGACGTTGATAGGCCGGGTGTGTAA |
| | GCGCAGCGATGCGTTGAGCTAACCGGTACTAATGAACCGTGAGGCTTA |
| | ACCTT (SEQ ID NO: 392) |
| rrlD | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGCGAACGGGGAGCAGCCCAGAGCCTGAATCAGTGTGTG |
| | TGTTAGTGGAAGCGTCTGGAAAGGCGCGCGATACAGGGTGACAGCCCC |
| | GTACACAAAAATGCACATGCTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCACGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAATTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACGCTTATGCGTTGTTGGGTAGGGGAGCGTTCTGTAAG |
| | CCTGCGAAGGTGTGCTGTGAGGCATGCTGGAGGTATCAGAAGTGCGAA |
| | TGCTGACATAAGTAACGATAAAGCGGGTGAAAAGCCCGCTCGCCGGAA |
| | GACCAAGGGTTCCTGTCCAACGTTAATCGGGGCAGGGTGAGTCGACCC |
| | CTAAGGCGAGGCCGAAAGGCGTAGTCGATGGGAAACAGGTTAATATTC |
| | CTGTACTTGGTGTTACTGCGAAGGGGGGACGGAGAAGGCTATGTTGGC |
| | CGGGCGACGGTTGTCCCGGTTTAAGCGTGTAGGCTGGTTTTCCAGGCAA |
| | ATCCGGAAAATCAAGGCTGAGGCGTGATGACGAGGCACTACGGTGCTG |
| | AAGCAACAAATGCCCTGCTTCCAGGAAAAGCCTCTAAGCATCAGGTAA |
| | CATCAAATCGTACCCCAAACCGACACAGGTGGTCAGGTAGAGAATACC |
| | AAGGCGCTTGAGAGAACTCGGGTGAAGGAACTAGGCAAAATGGTGCCG |
| | TAACTTCGGGAGAAGGCACGCTGATATGTAGGTGAAGCGACTTGCTCGT |
| | GGAGCTGAAATCAGTCGAAGATACCAGCTGGCTGCAACTGTTTATTAAA |
| | AACACAGCACTGTGCAAACACGAAAGTGGACGTATACGGTGTGACGCC |
| | TGCCCGGTGCCGGAAGGTTAATTGATGGGGTTAGCGCAAGCGAAGCTC |
| | TTGATCGAAGCCCCGGTAAACGGCGGCCGTAACTATAACGGTCCTAAG |
| | GTAGCGAAATTCCTTGTCGGGTAAGTTCCGACCTGCACGAATGGCGTAA |
| | TGATGGCCAGGCTGTCTCCACCCGAGACTCAGTGAAATTGAACTCGCTG |
| | TGAAGATGCAGTGTACCCGCGGCAAGACGGTAAGACCCCGTGAACCTT |
| | TACTATAGCTTGACACTGAACATTGAGCCTTGATGTGTAGGATAGGTGG |
| | GAGGCTTAGAAGTGTGGACGCCAGTCTGCATGGAGCCGACCTTGAAAT |
| | ACCACCCTTTAATGTTTGATGTTCTAACGTTGACCCGTAATCCGGGTTGC |
| | GGACAGTGTCTGGTGGGTAGTTTGACTGGGGCGGTCTCCTCCTAAAGAG |
| | TAACGGAGGAGCACGAAGGTTGGCTAATCCTGGTCGGACATCAGGAGG |
| | TTAGTGCAATGGCATAAGCCAGCTTGACTGCGAGCGTGACGGCGCGAG |
| | CAGGTGCGAAAGCAGGTCATAGTGATCCGGTGGTTCTGAATGGAAGGG |
| | CCATCGCTCAACGGATAAAAGGTACTCCGGGGATAACAGGCTGATACC |
| | GCCCAAGAGTTCATATCGACGGCGGTGTTTGGCACCTCGATGTCGGCTC |
| | ATCACATCCTGGGGCTGAAGTAGGTCCCAAGGGTGATGCTGTTCGCCAT |
| | TTAAAGTGGTACGCGAGCTGGGTTTAGAACGTCGTGAGACAGTTCGGTC |
| | CCTATCTGCCGTGGGCGCTGGAGAACTGAGGGGGGCTGCTCCTAGTACG |
| | AGAGGACCGGAGTGGACGCATCACTGGTGTTCGGGTTGTCATGCCAAT |
| | GGCACTGCCCGGTAGCTAAATGCGGAAGAGATAAGTGCTGAAAGCATC |
| | TAAGCACGAAACTTGCCCCGAGATGAGTTCTCCCTGACCCTTTAAGGGT |
| | CCTGAAGGAACGTTGAAGACGACGACGTTGATAGGCCGGGTGTGTAAG |
| | CGCAGCGATGCGTTGAGCTAACCGGTACTAATGAACCGTGAGGCTTAA |
| | CCTT (SEQ ID NO: 394) |
| rrlE | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGCGAACGGGGAGCAGCCCAGAGCCTGAATCAGTGTGTG |
| | TGTTAGTGGAAGCGTCTGGAAAGGCGCGCGATACAGGGTGACAGCCCC |
| | GTACACAAAAATGCACATGCTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCACGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAATTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACGCTTATGCGTTGTTGGGTAGGGGAGCGTTCTGTAAG |
| | CCTGCGAAGGTGTGCTGTGAGGCATGCTGGAGGTATCAGAAGTGCGAA |
| | TGCTGACATAAGTAACGATAAAGCGGGTGAAAAGCCCGCTCGCCGGAA |
| | GACCAAGGGTTCCTGTCCAACGTTAATCGGGGCAGGGTGAGTCGACCC |
| | CTAAGGCGAGGCCGAAAGGCGTAGTCGATGGGAAACAGGTTAATATTC |
| | CTGTACTTGGTGTTACTGCGAAGGGGGGACGGAGAAGGCTATGTTGGC |
| | CGGGCGACGGTTGTCCCGGTTTAAGCGTGTAGGCTGGTTTTCCAGGCAA |
| | ATCCGGAAAATCAAGGCTGAGGCGTGATGACGAGGCACTACGGTGCTG |
| | AAGCAACAAATGCCCTGCTTCCAGGAAAAGCCTCTAAGCATCAGGTAA |
| | CATCAAATCGTACCCCAAACCGACACAGGTGGTCAGGTAGAGAATACC |
| | AAGGCGCTTGAGAGAACTCGGGTGAAGGAACTAGGCAAAATGGTGCCG |
| | TAACTTCGGGAGAAGGCACGCTGATATGTAGGTGAGGTCCCTCGCGGA |
| | TGGAGCTGAAATCAGTCGAAGATACCAGCTGGCTGCAACTGTTTATTAA |
| | AAACACAGCACTGTGCAAACACGAAAGTGGACGTATACGGTGTGACGC |
| | CTGCCCGGTGCCGGAAGGTTAATTGATGGGGTTAGCGCAAGCGAAGCT |
| | CTTGATCGAAGCCCCGGTAAACGGCGGCCGTAACTATAACGGTCCTAA |
| | GGTAGCGAAATTCCTTGTCGGGTAAGTTCCGACCTGCACGAATGGCGTA |
| | ATGATGGCCAGGCTGTCTCCACCCGAGACTCAGTGAAATTGAACTCGCT |
| | GTGAAGATGCAGTGTACCCGCGGCAAGACGGTAAGACCCCGTGAACCT |
| | TTACTATAGCTTGACACTGAACATTGAGCCTTGATGTGTAGGATAGGTG |
| | GGAGGCTTTGAAGTGTGGACGCCAGTCTGCATGGAGCCGACCTTGAAA |
| | TACCACCCTTTAATGTTTGATGTTCTAACGTTGACCCGTAATCCGGGTTG |
| | CGGACAGTGTCTGGTGGGTAGTTTGACTGGGGCGGTCTCCTCCTAAAGA |
| | GTAACGGAGGAGCACGAAGGTTGGCTAATCCTGGTCGGACATCAGGAG |
| | GTTAGTGCAATGGCATAAGCCAGCTTGACTGCGAGCGTGACGGCGCGA |
| | GCAGGTGCGAAAGCAGGTCATAGTGATCCGGTGGTTCTGAATGGAAGG |
| | GCCATCGCTCAACGGATAAAAGGTACTCCGGGGATAACAGGCTGATAC |
| | CGCCCAAGAGTTCATATCGACGGCGGTGTTTGGCACCTCGATGTCGGCT |
| | CATCACATCCTGGGGCTGAAGTAGGTCCCAAGGGTATGGCTGTTCGCCA |
| | TTTAAAGTGGTACGCGAGCTGGGTTTAGAACGTCGTGAGACAGTTCGGT |
| | CCCTATCTGCCGTGGGCGCTGGAGAACTGAGGGGGGCTGCTCCTAGTAC |
| | GAGAGGACCGGAGTGGACGCATCACTGGTGTTCGGGTTGTCATGCCAA |
| | TGGCACTGCCCGGTAGCTAAATGCGGAAGAGATAAGTGCTGAAAGCAT |
| | CTAAGCACGAAACTTGCCCCGAGATGAGTTCTCCCTGACTCCTTGAGAG |
| | TCCTGAAGGAACGTTGAAGACGACGACGTTGATAGGCCGGGTGTGTAA |
| | GCGCAGCGATGCGTTGAGCTAACCGGTACTAATGAACCGTGAGGCTTA |
| | ACCTT (SEQ ID NO: 396) |
| rrlG | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGC GAACGGGGAGCAGCCCAGAGCCTGAATCAGTGTGTG |
| | TGTTAGTGGAAGCGTCTGGAAAGGCGCGCGATACAGGGTGACAGCCCC |
| | GTACACAAAAATGCACATGCTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCACGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAATTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACGCTTATGCGTTGTTGGGTAGGGGAGCGTTCTGTAAG |
| | CCTGCGAAGGTGTGCTGTGAGGCATGCTGGAGGTATCAGAAGTGCGAA |
| | TGCTGACATAAGTAACGATAAAGCGGGTGAAAAGCCCGCTCGCCGGAA |
| | GACCAAGGGTTCCTGTCCAACGTTAATCGGGGCAGGGTGAGTCGACCC |
| | CTAAGGCGAGGCCGAAAGGCGTAGTCGATGGGAAACAGGTTAATATTC |
| | CTGTACTTGGTGTTACTGCGAAGGGGGGACGGAGAAGGCTATGTTGGC |
| | CGGGCGACGGTTGTCCCGGTTTAAGCGTGTAGGCTGGTTTTCCAGGCAA |
| | ATCCGGAAAATCAAGGCTGAGGCGTGATGACGAGGCACTACGGTGCTG |
| | AAGCAACAAATGCCCTGCTTCCAGGAAAAGCCTCTAAGCATCAGGTAA |
| | CATCAAATCGTACCCCAAACCGACACAGGTGGTCAGGTAGAGAATACC |
| | AAGGCGCTTGAGAGAACTCGGGTGAAGGAACTAGGCAAAATGGTGCCG |
| | TAACTTCGGGAGAAGGCACGCTGATATGTAGGTGAGGTCCCTCGCGGA |
| | TGGAGCTGAAATCAGTCGAAGATACCAGCTGGCTGCAACTGTTTATTAA |
| | AAACACAGCACTGTGCAAACACGAAAGTGGACGTATACGGTGTGACGC |
| | CTGCCCGGTGCCGGAAGGTTAATTGATGGGGTTAGCGCAAGCGAAGCT |
| | CTTGATCGAAGCCCCGGTAAACGGCGGCCGTAACTATAACGGTCCTAA |
| | GGTAGCGAAATTCCTTGTCGGGTAAGTTCCGACCTGCACGAATGGCGTA |
| | ATGATGGCCAGGCTGTCTCCACCCGAGACTCAGTGAAATTGAACTCGCT |
| | GTGAAGATGCAGTGTACCCGCGGCAAGACGGTAAGACCCCGTGAACCT |
| | TTACTATAGCTTGACACTGAACATTGAGCCTTGATGTGTAGGATAGGTG |
| | GGAGGCTTTGAAGTGTGGACGCCAGTCTGCATGGAGCCGACCTTGAAA |
| | TACCACCCTTTAATGTTTGATGTTCTAACGTTGACCCGTAATCCGGGTTG |
| | CGGACAGTGTCTGGTGGGTAGTTTGACTGGGGCGGTCTCCTCCTAAAGA |
| | GTAACGGAGGAGCACGAAGGTTGGCTAATCCTGGTCGGACATCAGGAG |
| | GTTAGTGCAATGGCATAAGCCAGCTTGACTGCGAGCGTGACGGCGCGA |
| | GCAGGTGCGAAAGCAGGTCATAGTGATCCGGTGGTTCTGAATGGAAGG |
| | GCCATCGCTCAACGGATAAAAGGTACTCCGGGGATAACAGGCTGATAC |
| | CGCCCAAGAGTTCATATCGACGGCGGTGTTTGGCACCTCGATGTCGGCT |
| | CATCACATCCTGGGGCTGAAGTAGGTCCCAAGGGTATGGCTGTTCGCCA |
| | TTTAAAGTGGTACGCGAGCTGGGTTTAGAACGTCGTGAGACAGTTCGGT |
| | CCCTATCTGCCGTGGGCGCTGGAGAACTGAGGGGGGCTGCTCCTAGTAC |
| | GAGAGGACCGGAGTGGACGCATCACTGGTGTTCGGGTTGTCATGCCAA |
| | TGGCACTGCCCGGTAGCTAAATGCGGAAGAGATAAGTGCTGAAAGCAT |
| | CTAAGCACGAAACTTGCCCCGAGATGAGTTCTCCCTGACCCTTTAAGGG |
| | TCCTGAAGGAACGTTGAAGACGACGACGTTGATAGGCCGGGTGTGTAA |
| | GCGCAGCGATGCGTTGAGCTAACCGGTACTAATGAACCGTGAGGCTTA |
| | ACCTT (SEQ ID NO: 398) |
| rrlH | GGTTAAGCGACTAAGCGTACACGGTGGATGCCCTGGCAGTCAGAGGCG |
| A2058U | ATGAAGGACGTGCTAATCTGCGATAAGCGTCGGTAAGGTGATATGAAC |
| | CGTTATAACCGGCGATTTCCGAATGGGGAAACCCAGTGTGTTTCGACAC |
| | ACTATCATTAACTGAATCCATAGGTTAATGAGGCGAACCGGGGGAACT |
| | GAAACATCTAAGTACCCCGAGGAAAAGAAATCAACCGAGATTCCCCCA |
| | GTAGCGGCGAGCGAACGGGGAGGAGCCCAGAGCCTGAATCAGTGTGTG |
| | TGTTAGTGGAAGCGTCTGGAAAGGCGCGCGATACAGGGTGACAGCCCC |
| | GTACACAAAAATGCACATGCTGTGAGCTCGATGAGTAGGGCGGGACAC |
| | GTGGTATCCTGTCTGAATATGGGGGGACCATCCTCCAAGGCTAAATACT |
| | CCTGACTGACCGATAGTGAACCAGTACCGTGAGGGAAAGGCGAAAAGA |
| | ACCCCGGCGAGGGGAGTGAAAAAGAACCTGAAACCGTGTACGTACAAG |
| | CAGTGGGAGCATGCTTAGGCGTGTGACTGCGTACCTTTTGTATAATGGG |
| | TCAGCGACTTATATTCTGTAGCAAGGTTAACCGAATAGGGGAGCCGAA |
| | GGGAAACCGAGTCTTAACTGGGCGTTAAGTTGCAGGGTATAGACCCGA |
| | AACCCGGTGATCTAGCCATGGGCAGGTTGAAGGTTGGGTAACACTAAC |
| | TGGAGGACCGAACCGACTAATGTTGAAAAATTAGCGGATGACTTGTGG |
| | CTGGGGGTGAAAGGCCAATCAAACCGGGAGATAGCTGGTTCTCCCCGA |
| | AAGCTATTTAGGTAGCGCCTCGTGAACTCATCTCCGGGGGTAGAGCACT |
| | GTTTCGGCAAGGGGGTCATCCCGACTTACCAACCCGATGCAAACTGCGA |
| | ATACCGGAGAATGTTATCACGGGAGACACACGGCGGGTGCTAACGTCC |
| | GTCGTGAAGAGGGAAACAACCCAGACCGCCAGCTAAGGTCCCAAAGTC |
| | ATGGTTAAGTGGGAAACGATGTGGGAAGGCCCAGACAGCCAGGATGTT |
| | GGCTTAGAAGCAGCCATCATTTAAAGAAAGCGTAATAGCTCACTGGTC |
| | GAGTCGGCCTGCGCGGAAGATGTAACGGGGCTAAACCATGCACCGAAG |
| | CTGCGGCAGCGACGCTTATGCGTTGTTGGGTAGGGGAGCGTTCTGTAAG |
| | CCTGTGAAGGTGTACTGTGAGGTATGCTGGAGGTATCAGAAGTGCGAA |
| | TGCTGACATAAGTAACGATAAAGCGGGTGAAAAGCCCGCTCGCCGGAA |
| | GACCAAGGGTTCCTGTCCAACGTTAATCGGGGCAGGGTGAGTCGACCC |
| | CTAAGGCGAGGCCGAAAGGCGTAGTCGATGGGAAACAGGTTAATATTC |
| | CTGTACTTGGTGTTACTGCGAAGGGGGGACGGAGAAGGCTATGTTGGC |
| | CGGGCGACGGTTGTCCCGGTTTAAGCGTGTAGGCTGGTTTTCCAGGCAA |
| | ATCCGGAAAATCAAGGCTGAGGCGTGATGACGAGGCACTACGGTGCTG |
| | AAGCAACAAATGCCCTGCTTCCAGGAAAAGCCTCTAAGCATCAGGTAA |
| | CATCAAATCGTACCCCAAACCGACACAGGTGGTCAGGTAGAGAATACC |
| | AAGGCGCTTGAGAGAACTCGGGTGAAGGAACTAGGCAAAATGGTGCCG |
| | TAACTTCGGGAGAAGGCACGCTGATATGTAGGTGAAGCGACTTGCTCGT |
| | GGAGCTGAAATCAGTCGAAGATACCAGCTGGCTGCAACTGTTTATTAAA |
| | AACACAGCACTGTGCAAACACGAAAGTGGACGTATACGGTGTGACGCC |
| | TGCCCGGTGCCGGAAGGTTAATTGATGGGGTTAGCGCAAGCGAAGCTC |
| | TTGATCGAAGCCCCGGTAAACGGCGGCCGTAACTATAACGGTCCTAAG |
| | GTAGCGAAATTCCTTGTCGGGTAAGTTCCGACCTGCACGAATGGCGTAA |
| | TGATGGCCAGGCTGTCTCCACCCGAGACTCAGTGAAATTGAACTCGCTG |
| | TGAAGATGCAGTGTACCCGCGGCAAGACGGTAAGACCCCGTGAACCTT |
| | TACTATAGCTTGACACTGAACATTGAGCCTTGATGTGTAGGATAGGTGG |
| | GAGGCTTTGAAGTGTGGACGCCAGTCTGCATGGAGCCGACCTTGAAAT |
| | ACCACCCTTTAATGTTTGATGTTCTAACGTTGACCCGTAATCCGGGTTGC |
| | GGACAGTGTCTGGTGGGTAGTTTGACTGGGGCGGTCTCCTCCTAAAGAG |
| | TAACGGAGGAGCACGAAGGTTGGCTAATCCTGGTCGGACATCAGGAGG |
| | TTAGTGCAATGGCATAAGCCAGCTTGACTGCGAGCGTGACGGCGCGAG |
| | CAGGTGCGAAAGCAGGTCATAGTGATCCGGTGGTTCTGAATGGAAGGG |
| | CCATCGCTCAACGGATAAAAGGTACTCCGGGGATAACAGGCTGATACC |
| | GCCCAAGAGTTCATATCGACGGCGGTGTTTGGCACCTCGATGTCGGCTC |
| | ATCACATCCTGGGGCTGAAGTAGGTCCCAAGGGTATGGCTGTTCGCCAT |
| | TTAAAGTGGTACGCGAGCTGGGTTTAGAACGTCGTGAGACAGTTCGGTC |
| | CCTATCTGCCGTGGGCGCTGGAGAACTGAGGGGGGCTGCTCCTAGTACG |
| | AGAGGACCGGAGTGGACGCATCACTGGTGTTCGGGTTGTCATGCCAAT |
| | GGCACTGCCCGGTAGCTAAATGCGGAAGAGATAAGTGCTGAAAGCATC |
| | TAAGCACGAAACTTGCCCCGAGATGAGTTCTCCCTGACTCCTTGAGAGT |
| | CCTGAAGGAACGTTGAAGACGACGACGTTGATAGGCCGGGTGTGTAAG |
| | CGCAGCGATGCGTTGAGCTAACCGGTACTAATGAACCGTGAGGCTTAA |
| | CCTT (SEQ ID NO: 400) |

Next, the ErmC leader peptide, ermCL, was introduced ahead of the orthogonal sfGFP reporter, ensuring that reporter translation would be abrogated via erythromycin- and ermCL-dependent translational stalling (78) (FIG. 18B).

Reporter plasmid pAB140j8 was specifically employed, having the following nucleic acid sequence:

(SEQ ID NO: 384)
TTGAGACACAACGTGGCTTTCCATCAAAAAAATATTGACAACATAAAAAA
CTTTGTGTTATACTTGTGGAATTGTGAGCGGATAACAATTCTATATCTGT
TATTTTTTCCAACCACAGATCTATGGGCATTTTTAGTATTTTTGTAATCA
GCACAGTTCATTATCAACCAAACAAAAAATTAAGTGGTTATAATGAATCG
TTAATAAGCAAAATTCATTATAACCAAATTAGCAAAGGTGAAGAACTGTT
TACCGGCGTTGTGCCGATTCTGGTGGAACTGGATGGCGATGTGAACGGTC
ACAAATTCAGCGTGCGTGGTGAAGGTGAAGGCGATGCCACGATTGGCAAA
CTGACGCTGAAATTTATCTGCACCACCGGCAAACTGCCGGTGCCGTGGCC
GACGCTGGTGACCACCCTGACCTATGGCGTTCAGTGTTTTAGTCGCTATC
CGGATCACATGAAACGTCACGATTTCTTTAAATCTGCAATGCCGGAAGGC
TATGTGCAGGAACGTACGATTAGCTTTAAAGATGATGGCAAATATAAAAC
GCGCGCCGTTGTGAAATTTGAAGGCGATACCCTGGTGAACCGCATTGAAC
TGAAAGGCACGGATTTTAAAGAAGATGGCAATATCCTGGGCCATAAACTG
GAATACAACTTTAATAGCCATAATGTTTATATTACGGCGGATAAACAGAA
AAATGGCATCAAAGCGAATTTTACCGTTCGCCATAACGTTGAAGATGGCA
GTGTGCAGCTGGCAGATCATTATCAGCAGAATACCCCGATTGGTGATGGT
CCGGTGCTGCTGCCGGATAATCATTATCTGAGCACGCAGACCGTTCTGTC
TAAAGATCCGAACGAAAAAGGCACGCGGGACCACATGGTTCTGCACGAAT
ATGTGAATGCGGCAGGTATTATGTGGAGCCATCCGCAGTTCGAAAAATAA
GTCGACCGGCTGCTAACAAAGCCCGCGGCCGCTGAAGATCGATCTCGACG
AGTGAGAGAAGATTTTCAGCCTGATACAGATTAAATCAGAACGCAGAAGC
GGTCTGATAAAACAGAATTTGCCTGGCGGCAGTAGCGCGGTGGTCCCACC
TGACCCCATGCCGAACTCAGAAGTGAAACGCCGTAGCGCCGATGGTAGTG
TGGGGTCACCCCATGCGAGAGTAGGGAACTGCCAGGCATCAAATAAAACG
AAAGGCTCAGTCGAAAGACTGGGCCTTTCGTTTTATCTGTTGTTTGTCGG
TGAACGCTCTCCTGAGTAGGACAAATCCGCCGGGAGCGGATTTGAACGTT
GCGAAGCAACGGCCCGGAGGGTGGCGGGCAGGACGCCCGCCATAAACTGC
CAGGCATCAAATTAAGCAGAAGGCCATCCTGACGGATGGCCTTTTTGCGT
TTCTACAGAGCGTCAGACCCCTTAATAAGATGATCTTCTTGAGATCGTTT
TGGTCTGCGCGTAATCTCTTGCTCTGAAAACGAAAAAACCGCCTTGCAGG
GCGGTTTTTCGAAGGTTCTCTGAGCTACCAACTCTTTGAACCGAGGTAAC
TGGCTTGGAGGAGCGCAGTCACCAAAACTTGTCCTTTCAGTTTAGCCTTA
ACCGGCGCATGACTTCAAGACTAACTCCTCTAAATCAATTACCAGTGGCT
GCTGCCAGTGGTGCTTTTGCATGTCTTTCCGGGTTGGACTCAAGACGATA
GTTACCGGATAAGGCGCAGCGGTCGGACTGAACGGGGGGTTCGTGCATAC
AGTCCAGCTTGGAGCGAACTGCCTACCCGGAACTGAGTGTCAGGCGTGGA
ATGAGACAAACGCGGCCATAACAGCGGAATGACACCGGTAAACCGAAAGG
CAGGAACAGGAGAGCGCACGAGGGAGCCGCCAGGGGGAAACGCCTGGTAT
CTTTATAGTCCTGTCGGGTTTCGCCACCACTGATTTGAGCGTCAGATTTC
GTGATGCTTGTCAGGGGGGCGGAGCCTATGGAAAAACGGCTCAAGTCAGC
GTAATGCTCTGCCAGTGTTACAACCAATTAACCAATTCTGATTAGAAAAA
CTCATCGAGCATCAAATGAAACTGCAATTTATTCATATCAGGATTATCAA
TACCATATTTTTGAAAAAGCCGTTTCTGTAATGAAGGAGAAAACTCACCG
AGGCAGTTCCATAGGATGGCAAGATCCTGGTATCGGTCTGCGATTCCGAC
TCGTCCAACATCAATACAACCTATTAATTTCCCCTCGTCAAAAATAAGGT
TATCAAGTGAGAAATCACCATGAGTGACGACTGAATCCGGTGAGAATGGC
AAAAGCTTATGCATTTCTTTCCAGACTTGTTCAACAGGCCAGCCATTACG
CTCGTCATCAAAATCACTCGCATCAACCAAACCGTTATTCATTCGTGATT
GCGCCTGAGCGAGACGAAATACGCGATCGCTGTTAAAAGGACAATTACAA
ACAGGAATCGAATGCAACCGGCGCAGGAACACTGCCAGCGCATCAACAAT
ATTTTCACCTGAATCAGGATATTCTTCTAATACCTGGAATGCTGTTTTCC
CGGGGATCGCAGTGGTGAGTAACCATGCATCATCAGGAGTACGGATAAAA
TGCTTGATGGTCGGAAGAGGCATAAATTCCGTCAGCCAGTTTAGTCTGAC
CATCTCATCTGTAACATCATTGGCAACGCTACCTTTGCCATGTTTCAGAA
ACAACTCTGGCGCATCGGGCTTCCCATACAATCGATAGATTGTCGCACCT
GATTGCCCGACATTATCGCGAGCCCATTTATACCCATATAAATCAGCATC
CATGTTGGAATTTAATCGCGGCCTCGAGCAAGACGTTTCCCGTTGAATAT
GGCTCATAACACCCCTTGTATTACTGTTTATGTAAGCAGACAGTTTTATT
GTTCATGATGATATATTTTTATCTTGTGCAATGTAACATCAGAGATT

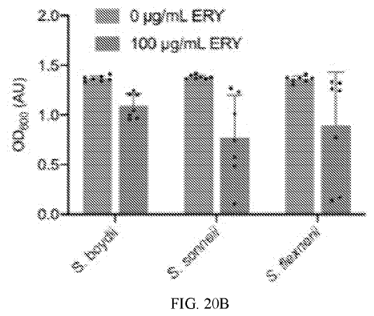

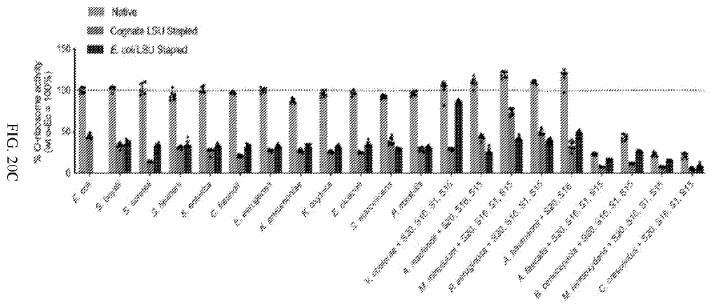

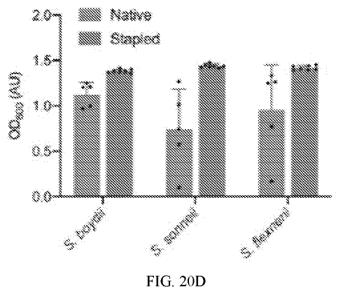

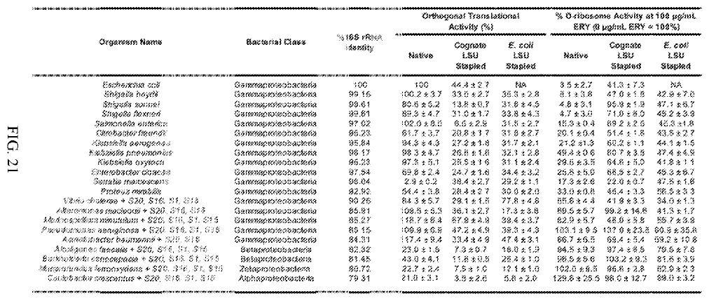

To validate this sensor, the erythromycin sensitivity of an episome-derived E. coli orthogonal ribosome encoding or lacking the identical A2058U mutation was assessed. A recently described stapled E. coli ribosome (d2d8) (7) that preferentially uses a covalently linked 23S rRNA, with or without the identical mutation was used as a control. Using this sensor/strain combination, it was observed that A2058U-LSUs showed no appreciable change in orthogonal translation upon erythromycin dosing (FIG. 18C). Conversely, unmutated LSUs showed a marked reduction in orthogonal translation in an erythromycin dose-dependent manner. Unstapled E. coli LSUs lacking the A2058U mutation re-sensitized S4246 cells to erythromycin, whereas the stapled counterpart did not (FIG. 18D), which indicated that plasmid-encoded LSUs co-assembled with genome-encoded SSUs and generated erythromycin-sensitive ribosomes incapable of translating essential E. coli genes (FIG. 20A).

This analysis was next extended to a set of 20 functional heterologous ribosomes. For heterologous ribosomes with high 16S sequence identity to E. coli (≥99.2%), a dramatic reduction in both sfGFP translation and cell viability was observed (FIGS. 18E, 20B and 21), demonstrating appreciable exchange between host and heterologous ribosomes. Notably, heterologous ribosomes bearing intermediate homology (92.9-97.0%) showed extensive reduction in sfGFP signal upon erythromycin treatment with no associated viability defect. These reductions in sfGFP signal were comparable to or greater than the corresponding effect on the d2d8 stapled E. coli ribosome, indicating a similar degree of association between cognate subunits. However, for more divergent heterologous ribosomes (79.3-90.3%), sfGFP signal decreased minimally upon erythromycin treatment, indicating that additional rRNA operon modifications or complementation with cognate factors is necessary to enable the preferential usage of the heterologous LSU.

To reduce association between heterologous and E. coli ribosomal subunits, the above rRNA stapling approach (7) was extended to the same 20 heterologous ribosomes, wherein the staple was extensible to rRNAs with high sequence identity (>99.2% 16S) to E. coli (FIGS. 20C to 20E). However, for most rRNAs, this approach did not increase erythromycin sensitivity. Therefore, it was concluded that the d2d8 linkers were not suitable for heterologous LSUs, and “hybrid” ribosomes were generated comprising heterologous SSUs stapled to E. coli LSUs. These hybrid ribosomes varied considerably in erythromycin sensitivity, which indicated that implemented rRNA linkers required independent optimization for each heterologous ribosome. Collectively, these data demonstrated that intermediately divergent heterologous SSUs preferentially associated with cognate LSUs in E. coli.

Thus, a library of 34 heterologous ribosomes derived from species across a broad phylogenetic range has been constructed herein and expressed in E. coli. The functionality of each of these ribosomes has been evaluated using both Δ7 strain complementation and orthogonal translation, with a high degree of correlation observed between the two assays. Remarkably, replacement of intergenic sequences with those of E. coli, as well as supplementation with only a small subset of r-proteins (S20, S16, S1, S15), significantly improved expression from orthogonal heterologous rRNAs. While o-rRNAs with high sequence identity (up to 96.2% 16S rRNA sequence identity to E. coli) natively translated superfolder GFP (sfGFP) at robust levels, substitution of intergenic sequences allowed for o-rRNAs as divergent as P. mirabilis (92.9%) to translate the orthogonal transcript at levels similar to the E. coli o-rRNA. Supplementation with r-proteins S20 and S16 allowed for similarly robust levels of translation from o-rRNA derived from A. baumannii (84.3%). More remarkably, using a more extensive set of r-proteins, heterologous translation from o-rRNAs as diverged as E. faecalis (76.1%) was achieved in the instant E. coli system. Further, an erythromycin-dependent reporter system was developed, which demonstrated that a subset of heterologous SSUs preferentially associated with their cognate LSUs.

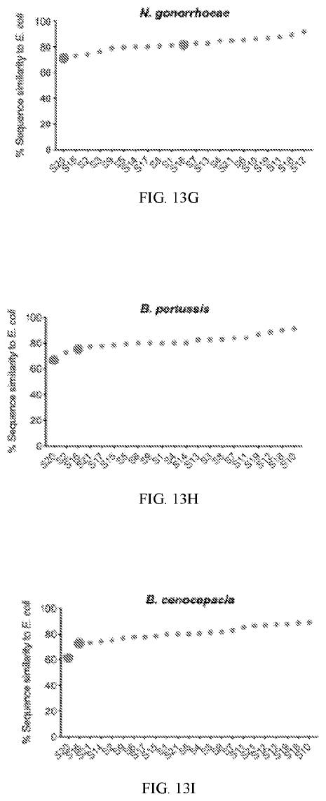

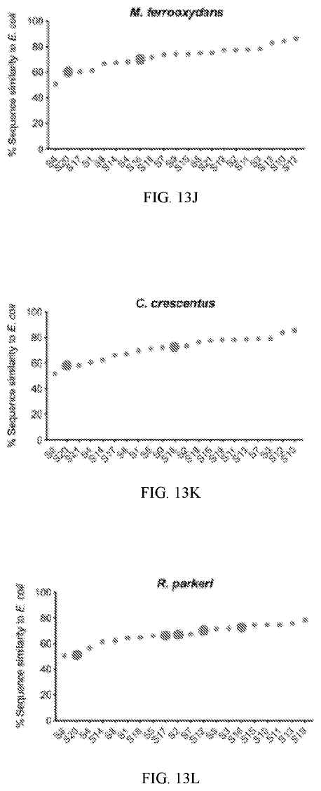

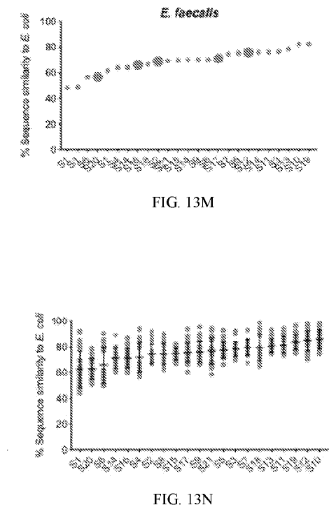

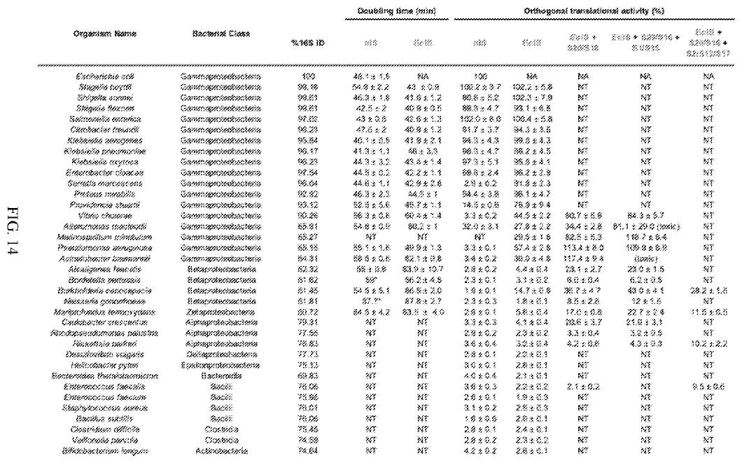

Collectively, the instant disclosure has established orthogonal translation as a viable alternative to Δ7 complementation for evaluating the function of heterologous rRNAs and has provided generalizable strategies for enhancing heterologous rRNA function. Notably, of the four r-proteins found to be broadly important for o-rRNA function, only two (S1 and S16) have been found to be essential for viability in E. coli via gene knockout (45, 46), which indicates that essentiality cannot serve as a predictor of crucial factors enhancing o-rRNA function. It was therefore sought herein to determine whether rules for predicting r-proteins necessary for complementing heterologous o-rRNAs could be derived. Using sequence similarities of heterologous SSU r-proteins to their E. coli homologues, it was discovered that r-proteins empirically found to be crucial for complementation (S20, S16, and S15) tended to be those with the lowest sequence similarity (FIGS. 13A-13N). All three aforementioned proteins regulate the earliest stages of 30S assembly (79). In the case of S20 and S16, low sequence similarity is likely due to their roles as primary binders to the rRNA (44, 45, 79). In the case of S1, it often interacts with mRNAs in proximity to the RBS during translational initiation (80, 81), indicating that it mediates correct RBS/antiRBS interactions using noncanonical (orthogonal) pairs. An approach based on sequence divergence is therefore provided as a guide for predicting r-proteins necessary for complementation of heterologous rRNAs.

It is therefore contemplated herein that heterologous rRNAs—specifically those of relevance to human health—can be expressed in E. coli for the high-throughput discovery of ribosome-targeting antibiotics. In particular, in certain embodiments, screening can be performed in the heterologous rRNA and/or r-protein systems that are disclosed herein, to identify antibiotics that are selective to ribosomes of pathogens, while doing less or no damage to ribosomes of commensal micro-organisms. Optionally, such screening assays can be multiplexed, thereby allowing for direct comparisons to be made between ribosomes of pathogenic microbes and ribosomes of non-pathogenic microbes (e.g., ribosomes of commensal microbes). More broadly, the strategies described herein can be used in the development of increasingly divergent ribosomes that limit interaction with host cells, yielding more orthogonal components for engineered variations of the central dogma. Finally, the heterologous ribosomes described infra are contemplated to serve as alternative starting points for the discovery and evolution of novel translational properties. Through the use of such diverse ribosomes, synthetic biologists can now take advantage of and likely repurpose the myriad functionalities for which bacteria have evolved their ribosomes.

Expressly contemplated applications of the compositions and methods of the instant disclosure therefore include, without limitation, the following:

(1) Scalable, and potentially multiplexed, discovery of molecules that selectively inhibit ribosomes of pathogenic bacteria, optionally in direct comparison to ribosomes of commensal bacteria: In an exemplary application, an orthogonal, heterologous ribosome is measured via a single reporter in E. coli. Combinations of pathogenic ribosomes and reporters are mixed in 96-well plate format. Combinations of commensal ribosomes and reporters are also optionally mixed in the same or parallel 96-well plate format. Test compound is added to each well. Relative decrease of reporter activity in a single well is considered a “hit” (putative specific inhibitor of bacterial translation), where a molecule that hits pathogenic ribosomes while having fewer or no hits to commensal ribosomes is considered a pathogen-selective “hit” as compared to commensal strains. Such a platform obviates inefficiencies traditionally associated with antimicrobial small-molecule screens (i.e., variable bacterial growth conditions, biohazard concerns).

(2) Prediction of resistance alleles (optionally to newly discovered small molecules): predicted resistance alleles can be introduced via PCR onto the rRNA of a heterologous ribosome. As for (1) above, test compounds can be screened against mutated ribosomes.

(3) Engineered heterologous ribosomes for enhanced bioproduction capabilities: heterologous ribosomes putatively share fewer resources with the E. coli host cell than native ribosomes. Heterologous orthogonal ribosomes may be directed towards the production of biomolecules for industry or pharmaceuticals.

Certain aspects of the instant disclosure contemplate use of the exemplary sequences presented in Tables 2-4 below.

Exemplified E. coli Intergenic Sequences

| Name | Sequence |
|---|---|
| Pre-16S | 5′-ACTGCTCTTTAACAATTTATCAGACAATCTGTGTG |
| | GGCACTCGAAGATACGGATTCTTAACGTCGCAAGACGA |
| | AAAATGAATACCAAGTCTCAAGAGTGAACACGTAATTC |
| | ATTACGAAGTTTAATTCTTTGAGCGTCAAACTTTT-3′ |
| | (SEQ ID NO: 410) |
| 16Sto 23S | 5′-CCTTAAAGAAGCGTACTTTGTAGTGCTCACACAGA |
| | TTGTCTGATAGAAAGTGAAAAGCAAGGCGTTTACGCGT |
| | TGGGAGTGAGGCTGAAGAGAATAAGGCCGTTCGCTTTC |
| | TATTAATGAAAGCTCACCCTACACGAAAATATCACGCA |
| | ACGCGTGATAAGCAATTTTCGTGTCCCCTTCGTCTAGA |
| | GGCCCAGGACACCGCCCTTTCACGGCGGTAACAGGGGT |
| | TCGAATCCCCTAGGGGACGCCACTTGCTGGTTTGTGAG |
| | TGAAAGTCGCCGACCTTAATATCTCAAAACTCATCTTC |
| | GGGTGATGTTTGAGATATTTGCTCTTTAAAAATCTGGA |
| | TCAAGCTGAAAATTGAAACACTGAACAACGAGAGTTGT |
| | TCGTGAGTCTCTCAAATTTTCGCAACACGATGATGAAT |
| | CGAAAGAAACATCTTCGGGTTGTGA-3′ |
| | (SEQ ID NO: 411) |
| 235 to 5S | 5′-ACAACGCCGAAGCTGTTTTGGCGGATGAGAGAAGA |
| | TTTTCAGCCTGATACAGATTAAATCAGAACGCAGAAGC |
| | GGTCTGATAAAACAGAATT-3′ |
| | (SEQ ID NO: 412) |
| Post-5S | 5′-CAAATAAAACGAAAGGCTCAGTCGAAAGACTGGGC |
| | CTTTCGTTTTATCTGTTGTTTGTCGGTGAACGCTCTCC |
| | TGAGTAGGACAAATCCGCCGGGAGCGGATTTGAACGTT |
| | GCGAAGCAACGGCCCGGAGGGTGGCGGGCAGGACGCCC |
| | GCCATAAACTGCCAGGCATCAAATTAAGCAGAAGGCCA |
| | TCCTGACGGATGGCCTTTTTGCGTTTCTACAAACTCTT |
| | CCTGTCGTCATATCTACAAGCC-3′ |
| | (SEQ ID NO: 413) |

TABLE 3
Exemplified and Expressly Contemplated 16S, 23Sand SSrRNA Coding Sequences

| Name | Sequence |
|---|---|
| A. baumannn 16S | 5′-TAACTGAAGAGTTTGATCATGGCTCAGATTGAACGCTGGCGGCAGGCTTAACACATGCAA |
| (contains O-antiRBS) | GTCGAGCGGGGGAAGGTAGCTTGCTACCGGACCTAGCGGCGGACGGGTGAGTAATGCTTAGGA |
| | ATCTGCCTATTAGTGGGGGACAACATCTCGAAAGGGATGCTAATACCGCATACGTCCTACGGG |
| | AGAAAGCAGGGGATCTTCGGACCTTGCGCTAATAGATGAGCCTAAGTCGGATTAGCTAGTTGG |
| | TGGGGTAAAGGCCTACCAAGGCGACGATCTGTAGCGGGTCTGAGAGGATGATCCGCCACACTG |
| | GGACTGAGACACGGCCCAGACTCCTACGGGAGGCAGCAGTGGGGAATATTGGACAATGGGCGC |
| | AAGCCTGATCCAGCCATGCCGCGTGTGTGAAGAAGGCCTTATGGTTGTAAAGCACTTTAAGCG |
| | AGGAGGAGGCTACTTTAGATAATACCTAGAGATAGTGGACGTTACTCGCAGAATAAGCACCGG |
| | CTAACTCTGTGCCAGCAGCCGCGGTAATACAGAGGGTGCAAGCGTTAATCGGATTTACTGGGC |
| | GTAAAGCGCGCGTAGGCGGCTAATTAAGTCAAATGTGAAATCCCCGAGCTTAACTTGGGAATT |
| | GCATTCGATACTGGTTAGCTAGAGTGTGGGAGAGGATGGTAGAATTCCAGGTGTAGCGGTGAA |
| | ATGCGTAGAGATCTGGAGGAATACCGATGGCGAAGGCAGCCATCTGGCCTAACACTGACGCTG |
| | AGGTGCGAAAGCATGGGGAGCAAACAGGATTAGATACCCTGGTAGTCCATGCCGTAAACGATG |
| | TCTACTAGCCGTTGGGGCCTTTGAGGCTTTAGTGGCGCAGCTAACGCGATAAGTAGACCGCCT |
| | GGGGAGTACGGTCGCAAGACTAAAACTCAAATGAATTGACGGGGGCCCGCACAAGCGGTGGAG |
| | CATGTGGTTTAATTCGATGCAACGCGAAGAACCTTACCTGGCCTTGACATAGTAAGAACTTTC |
| | CAGAGATGGATTGGTGCCTTCGGGAACTTACATACAGGTGCTGCATGGCTGTCGTCAGCTCGT |
| | GTCGTGAGATGTTGGGTTAAGTCCCGCAACGAGCGCAACCCTTTTCCTTATTTGCCAGCGAGT |
| | AATGTCGGGAACTTTAAGGATACTGCCAGTGACAAACTGGAGGAAGGCGGGGACGACGTCAAG |
| | TCATCATGGCCCTTACGGCCAGGGCTACACACGTGCTACAATGGTCGGTACAAAGGGTTGCTA |
| | CACAGCGATGTGATGCTAATCTCAAAAAGCCGATCGTAGTCCGGATTGGAGTCTGCAACTCGA |
| | CTCCATGAAGTCGGAATCGCTAGTAATCGCGGATCAGAATGCCGCGGTGAATACGTTCCCGGG |
| | CCTTGTACACACCGCCCGTCACACCATGGGAGTTTGTTGCACCAGAAGTAGCTAGCCTAACTG |
| | CAAAGAGGGCGGTTACCACGGTGTGGCCGATGACTGGGGTGAAGTCGTAACAAGGTAGCCGTA |
| | GGGGAACCTGCGGCTGGATCATGTGGTAT-3′ (SEQ ID NO: 414) |
| V. cholerae 16S | 5′-TAATTGAAGAGTTTGATCATGGCTCAGATTGAACGCTGGCGGCAGGCCTAACACATGCAA |
| (contains O-antiRBS) | GTCGAGCGGCAGCACAGAGGAACTTGTTCCTTGGGTGGCGAGCGGCGGACGGGTGAGTAATGC |
| | CTGGGAAATTGCCCGGTAGAGGGGGATAACCATTGGAAACGATGGCTAATACCGCATAACCTC |
| | GCAAGAGCAAAGCAGGGGACCTTCGGGCCTTGCGCTACCGGATATGCCCAGGTGGGATTAGCT |
| | AGTTGGTGAGGTAAGGGCTCACCAAGGCGACGATCCCTAGCTGGTCTGAGAGGATGATCAGCC |
| | ACACTGGAACTGAGACACGGTCCAGACTCCTACGGGAGGCAGCAGTGGGGAATATTGCACAAT |
| | GGGCGCAAGCCTGATGCAGCCATGCCGCGTGTATGAAGAAGGCCTTCGGGTTGTAAAGTACTT |
| | TCAGTAGGGAGGAAGGTGGTTAAGTTAATACCTTAATCATTTGACGTTACCTACAGAAGAAGC |
| | ACCGGCTAACTCCGTGCCAGCAGCCGCGGTAATACGGAGGGTGCAAGCGTTAATCGGAATTAC |
| | TGGGCGTAAAGCGCATGCAGGTGGTTTGTTAAGTCAGATGTGAAAGCCCTGGGCTCAACCTAG |
| | GAATCGCATTTGAAACTGACAAGCTAGAGTACTGTAGAGGGGGGTAGAATTTCAGGTGTAGCG |
| | GTGAAATGCGTAGAGATCTGAAGGAATACCGGTGGCGAAGGCGGCCCCCTGGACAGATACTGA |
| | CACTCAGATGCGAAAGCGTGGGGAGCAAACAGGATTAGATACCCTGGTAGTCCACGCCGTAAA |
| | CGATGTCTACTTGGAGGTTGTGACCTAGAGTCGTGGCTTTCGGAGCTAACGCGTTAAGTAGAC |
| | CGCCTGGGGAGTACGGTCGCAAGATTAAAACTCAAATGAATTGACGGGGGCCCGCACAAGCGG |
| | TGGAGCATGTGGTTTAATTCGATGCAACGCGAAGAACCTTACCTACTCTTGACATCCAGAGAA |
| | TCTAGCGGAGACGCTGGAGTGCCTTCGGGAGCTCTGAGACAGGTGCTGCATGGCTGTCGTCAG |
| | CTCGTGTTGTGAAATGTTGGGTTAAGTCCCGCAACGAGCGCAACCCTTATCCTTGTTTGCCAG |
| | CACGTAATGGTGGGAACTCCAGGGAGACTGCCGGTGATAAACCGGAGGAAGGTGGGGACGACG |
| | TCAAGTCATCATGGCCCTTACGAGTAGGGCTACACACGTGCTACAATGGCGTATACAGAGGGC |
| | AGCGATACCGCGAGGTGGAGCGAATCTCACAAAGTACGTCGTAGTCCGGATTGGAGTCTGCAA |
| | CTCGACTCCATGAAGTCGGAATCGCTAGTAATCGCAAATCAGAATGTTGCGGTGAATACGTTC |
| | CCGGGCCTTGTACACACCGCCCGTCACACCATGGGAGTGGGCTGCAAAAGAAGCAGGTAGTTT |
| | AACCTTCGGGAGGACGCTTGCCACTTTGTGGTTCATGACTGGGGTGAAGTCGTAACAAGGTAG |
| | CGCTAGGGGAACCTGGCGCTGGATCATGTGGTTA-3′ (SEQ ID NO: 415) |
| P. aeruginosa 16S | 5′-GAACTGAAGAGTTTGATCATGGCTCAGATTGAACGCTGGCGGCAGGCCTAACACATGCAA |
| (contains O-antiRBS) | GTCGAGCGGATGAAGGGAGCTTGCTCCTGGATTCAGCGGCGGACGGGTGAGTAATGCCTAGGA |
| | ATCTGCCTGGTAGTGGGGGATAACGTCCGGAAACGGGCGCTAATACCGCATACGTCCTGAGGG |
| | AGAAAGTGGGGGATCTTCGGACCTCACGCTATCAGATGAGCCTAGGTCGGATTAGCTAGTTGG |
| | TGGGGTAAAGGCCTACCAAGGCGACGATCCGTAACTGGTCTGAGAGGATGATCAGTCACACTG |
| | GAACTGAGACACGGTCCAGACTCCTACGGGAGGCAGCAGTGGGGAATATTGGACAATGGGCGA |
| | AAGCCTGATCCAGCCATGCCGCGTGTGTGAAGAAGGTCTTCGGATTGTAAAGCACTTTAAGTT |
| | GGGAGGAAGGGCAGTAAGTTAATACCTTGCTGTTTTGACGTTACCAACAGAATAAGCACCGGC |
| | TAACTTCGTGCCAGCAGCCGCGGTAATACGAAGGGTGCAAGCGTTAATCGGAATTACTGGGCG |
| | TAAAGCGCGCGTAGGTGGTTCAGCAAGTTGGATGTGAAATCCCCGGGCTCAACCTGGGAACTG |
| | CATCCAAAACTACTGAGCTAGAGTACGGTAGAGGGTGGTGGAATTTCCTGTGTAGCGGTGAAA |
| | TGCGTAGATATAGGAAGGAACACCAGTGGCGAAGGCGACCACCTGGACTGATACTGACACTGA |
| | GGTGCGAAAGCGTGGGGAGCAAACAGGATTAGATACCCTGGTAGTCCACGCCGTAAACGATGT |
| | CGACTAGCCGTTGGGATCCTTGAGATCTTAGTGGCGCAGCTAACGCGATAAGTCGACCGCCTG |
| | GGGAGTACGGCCGCAAGGTTAAAACTCAAATGAATTGACGGGGGCCCGCACAAGCGGTGGAGC |
| | ATGTGGTTTAATTCGAAGCAACGCGAAGAACCTTACCTGGCCTTGACATGCTGAGAACTTTCC |
| | AGAGATGGATTGGTGCCTTCGGGAACTCAGACACAGGTGCTGCATGGCTGTCGTCAGCTCGTG |
| | TCGTGAGATGTTGGGTTAAGTCCCGTAACGAGCGCAACCCTTGTCCTTAGTTACCAGCACCTC |
| | GGGTGGGCACTCTAAGGAGACTGCCGGTGACAAACCGGAGGAAGGTGGGGATGACGTCAAGTC |
| | ATCATGGCCCTTACGGCCAGGGCTACACACGTGCTACAATGGTCGGTACAAAGGGTTGCCAAG |
| | CCGCGAGGTGGAGCTAATCCCATAAAACCGATCGTAGTCCGGATCGCAGTCTGCAACTCGACT |
| | GCGTGAAGTCGGAATCGCTAGTAATCGTGAATCAGAATGTCACGGTGAATACGTTCCCGGGCC |
| | TTGTACACACCGCCCGTCACACCATGGGAGTGGGTTGCTCCAGAAGTAGCTAGTCTAACCGCA |
| | AGGGGGACGGTTACCACGGAGTGATTCATGACTGGGGTGAAGTCGTAACAAGGTAGCCGTAGG |
| | GGAACCTGCGGCTGGATCATGTGGTTA-3′ (SEQ ID NO: 416) |
| S. marcescens 16S | 5′-TAATTGAAGAGTTTGATCATGGCTCAGATTGAACGCTGGCGGCAGGCTTAACACATGCAA |
| (contains O-antiRBS) | GTCGAGCGGTAGCACAGGGGAGCTTGCTCCCTGGGTGACGAGCGGCGGACGGGTGAGTAATGT |
| | CTGGGAAACTGCCTGATGGAGGGGGATAACTACTGGAAACGGTAGCTAATACCGCATAACGTC |
| | GCAAGACCAAAGAGGGGGACCTTCGGGCCTCTTGCCATCAGATGTGCCCAGATGGGATTAGCT |
| | AGTAGGTGGGGTAATGGCTCACCTAGGCGACGATCCCTAGCTGGTCTGAGAGGATGACCAGCC |
| | ACACTGGAACTGAGACACGGTCCAGACTCCTACGGGAGGCAGCAGTGGGGAATATTGCACAAT |
| | GGGCGCAAGCCTGATGCAGCCATGCCGCGTGTGTGAAGAAGGCCTTCGGGTTGTAAAGCACTT |
| | TCAGCGAGGAGGAAGGTGGTGAGCTTAATACGCTCATCAATTGACGTTACTCGCAGAAGAAGC |
| | ACCGGCTAACTCCGTGCCAGCAGCCGCGGTAATACGGAGGGTGCAAGCGTTAATCGGAATTAC |
| | TGGGCGTAAAGCGCACGCAGGCGGTTTGTTAAGTCAGATGTGAAATCCCCGGGCTCAACCTGG |
| | GAACTGCATTTGAAACTGGCAAGCTAGAGTCTCGTAGAGGGGGGTAGAATTCCAGGTGTAGCG |
| | GTGAAATGCGTAGAGATCTGGAGGAATACCGGTGGCGAAGGCGGCCCCCTGGACGAAGACTGA |
| | CGCTCAGGTGCGAAAGCGTGGGGAGCAAACAGGATTAGATACCCTGGTAGTCCACGCTGTAAA |
| | CGATGTCGATTTGGAGGTTGTGCCCTTGAGGCGTGGCTTCCGGAGCTAACGCGTTAAATCGAC |
| | CGCCTGGGGAGTACGGCCGCAAGGTTAAAACTCAAATGAATTGACGGGGGCCCGCACAAGCGG |
| | TGGAGCATGTGGTTTAATTCGATGCAACGCGAAGAACCTTACCTACTCTTGACATCCAGAGAA |
| | CTTAGCAGAGATGCTTTGGTGCCTTCGGGAACTCTGAGACAGGTGCTGCATGGCTGTCGTCAG |
| | CTCGTGTTGTGAAATGTTGGGTTAAGTCCCGCAACGAGCGCAACCCTTATCCTTTGTTGCCAG |
| | CGGTTCGGCCGGGAACTCAAAGGAGACTGCCAGTGATAAACTGGAGGAAGGTGGGGATGACGT |
| | CAAGTCATCATGGCCCTTACGAGTAGGGCTACACACGTGCTACAATGGCATATACAAAGAGAA |
| | GCGACCTCGCGAGAGCAAGCGGACCTCATAAAGTATGTCGTAGTCCGGATTGGAGTCTGCAAC |
| | TCGACTCCATGAAGTCGGAATCGCTAGTAATCGTAGATCAGAATGCTACGGTGAATACGTTCC |
| | CGGGCCTTGTACACACCGCCCGTCACACCATGGGAGTGGGTTGCAAAAGAAGTAGGTAGCTTA |
| | ACCTTCGGGAGGGCGCTTACCACTTTGTGATTCATGACTGGGGTGAAGTCGTAACAAGGTAAC |
| | CGTAGGGGAACCTGCGGTTGGATCATGTGGTTA-3′ (SEQ ID NO: 417) |
| S. enterica 16S | 5′-AAATTGAAGAGTTTGATCATGGCTCAGATTGAACGCTGGCGGCAGGCCTAACACATGCAA |
| (contains O-antiRBS) | GTCGAACGGTAACAGGAAGCAGCTTGCTGCTTCGCTGACGAGTGGCGGACGGGTGAGTAATGT |
| | CTGGGAAACTGCCTGATGGAGGGGGATAACTACTGGAAACGGTGGCTAATACCGCATAACGTC |
| | GCAAGACCAAAGAGGGGGACCTTCGGGCCTCTTGCCATCAGATGTGCCCAGATGGGATTAGCT |
| | AGTTGGTGAGGTAACGGCTCACCAAGGCGACGATCCCTAGCTGGTCTGAGAGGATGACCAGCC |
| | ACACTGGAACTGAGACACGGTCCAGACTCCTACGGGAGGCAGCAGTGGGGAATATTGCACAAT |
| | GGGCGCAAGCCTGATGCAGCCATGCCGCGTGTATGAAGAAGGCCTTCGGGTTGTAAAGTACTT |
| | TCAGCGGGGAGGAAGGTGTTGTGGTTAATAACCGCAGCAATTGACGTTACCCGCAGAAGAAGC |
| | ACCGGCTAACTCCGTGCCAGCAGCCGCGGTAATACGGAGGGTGCAAGCGTTAATCGGAATTAC |
| | TGGGCGTAAAGCGCACGCAGGCGGTCTGTCAAGTCGGATGTGAAATCCCCGGGCTCAACCTGG |
| | GAACTGCATTCGAAACTGGCAGGCTTGAGTCTTGTAGAGGGGGGTAGAATTCCAGGTGTAGCG |
| | GTGAAATGCGTAGAGATCTGGAGGAATACCGGTGGCGAAGGCGGCCCCCTGGACAAAGACTGA |
| | CGCTCAGGTGCGAAAGCGTGGGGAGCAAACAGGATTAGATACCCTGGTAGTCCACGCCGTAAA |
| | CGATGTCTACTTGGAGGTTGTGCCCTTGAGGCGTGGCTTCCGGAGCTAACGCGTTAAGTAGAC |
| | CGCCTGGGGAGTACGGCCGCAAGGTTAAAACTCAAATGAATTGACGGGGGCCCGCACAAGCGG |
| | TGGAGCATGTGGTTTAATTCGATGCAACGCGAAGAACCTTACCTGGTCTTGACATCCACAGAA |
| | CTTTCCAGAGATGGATTGGTGCCTTCGGGAACTGTGAGACAGGTGCTGCATGGCTGTCGTCAG |
| | CTCGTGTTGTGAAATGTTGGGTTAAGTCCCGCAACGAGCGCAACCCTTATCCTTTGTTGCCAG |
| | CGATTAGGTCGGGAACTCAAAGGAGACTGCCAGTGATAAACTGGAGGAAGGTGGGGATGACGT |
| | CAAGTCATCATGGCCCTTACGACCAGGGCTACACACGTGCTACAATGGCGCATACAAAGAGAA |
| | GCGACCTCGCGAGAGCAAGCGGACCTCATAAAGTGCGTCGTAGTCCGGATTGGAGTCTGCAAC |
| | TCGACTCCATGAAGTCGGAATCGCTAGTAATCGTGGATCAGAATGCCACGGTGAATACGTTCC |
| | CGGGCCTTGTACACACCGCCCGTCACACCATGGGAGTGGGTTGCAAAAGAAGTAGGTAGCTTA |
| | ACCTTCGGGAGGGCGCTTACCACTTTGTGATTCATGACTGGGGTGAAGTCGTAACAAGGTAAC |
| | CGTAGGGGAACCTGCGGTTGGATCATGTGGTTA-3′ (SEQ ID NO: 418) |
| N. gonohhhoeae 16S | 5′-GAACATAAGAGTTTGATCCTGGCTCAGATTGAACGCTGGCGGCATGCTTTACACATGCAA |
| (contains O-antiRBS) | GTCGGACGGCAGCACAGGGAAGCTTGCTTCTCGGGTGGCGAGTGGCGAACGGGTGAGTAACAT |
| | ATCGGAACGTACCGGGTAGCGGGGGATAACTGATCGAAAGATCAGCTAATACCGCATACGTCT |
| | TGAGAGGGAAAGCAGGGGACCTTCGGGCCTTGCGCTATCCGAGCGGCCGATATCTGATTAGCT |
| | GGTTGGCGGGGTAAAGGCCCACCAAGGCGACGATCAGTAGCGGGTCTGAGAGGATGATCCGCC |
| | ACACTGGGACTGAGACACGGCCCAGACTCCTACGGGAGGCAGCAGTGGGGAATTTTGGACAAT |
| | GGGCGCAAGCCTGATCCAGCCATGCCGCGTGTCTGAAGAAGGCCTTCGGGTTGTAAAGGACTT |
| | TTGTCAGGGAAGAAAAGGCCGTTGCCAATATCGGCGGCCGATGACGGTACCTGAAGAATAAGC |
| | ACCGGCTAACTACGTGCCAGCAGCCGCGGTAATACGTAGGGTGCGAGCGTTAATCGGAATTAC |
| | TGGGCGTAAAGCGGGCGCAGACGGTTACTTAAGCAGGATGTGAAATCCCCGGGCTCAACCCGG |
| | GAACTGCGTTCTGAACTGGGTGACTCGAGTGTGTCAGAGGGAGGTGGAATTCCACGTGTAGCA |
| | GTGAAATGCGTAGAGATGTGGAGGAATACCGATGGCGAAGGCAGCCTCCTGGGATAACACTGA |
| | CGTTCATGTCCGAAAGCGTGGGTAGCAAACAGGATTAGATACCCTGGTAGTCCACGCCCTAAA |
| | CGATGTCAATTAGCTGTTGGGCAACTTGATTGCTTGGTAGCGTAGCTAACGCGTGAAATTGAC |
| | CGCCTGGGGAGTACGGTCGCAAGATTAAAACTCAAAGGAATTGACGGGGACCCGCACAAGCGG |
| | TGGATGATGTGGATTAATTCGATGCAACGCGAAGAACCTTACCTGGTTTTGACATGTGCGGAA |
| | TCCTCCGGAGACGGAGGAGTGCCTTCGGGAGCCGTAACACAGGTGCTGCATGGCTGTCGTCAG |
| | CTCGTGTCGTGAGATGTTGGGTTAAGTCCCGCAACGAGCGCAACCCTTGTCATTAGTTGCCAT |
| | CATTCGGTTGGGCACTCTAATGAGACTGCCGGTGACAAGCCGGAGGAAGGTGGGGATGACGTC |
| | AAGTCCTCATGGCCCTTATGACCAGGGCTTCACACGTCATACAATGGTCGGTACAGAGGGTAG |
| | CCAAGCCGCGAGGCGGAGCCAATCTCACAAAACCGATCGTAGTCCGGATTGCACTCTGCAACT |
| | CGAGTGCATGAAGTCGGAATCGCTAGTAATCGCAGGTCAGCATACTGCGGTGAATACGTTCCC |
| | GGGTCTTGTACACACCGCCCGTCACACCATGGGAGTGGGGGATACCAGAAGTAGGTAGGGTAA |
| | CCGCAAGGAGTCCGCTTACCACGGTATGCTTCATGACTGGGGTGAAGTCGTAACAAGGTAGCC |
| | GTAGGGGAACCTGCGGCTGGATCATGTGGTTT-3′ (SEQ ID NO: 419) |
| A. baumannn 23S | 5′-AGTCAAGTAATTAAGTGCATGTGGTGGATGCCTTGGCAGTCAGAGGCGATGAAAGACGTG |
| | ATAGCCTGCGAAAAGCTCCGGGGAGGCGGCAAATATCCTTTGATCCGGAGATGTCTGAATGGG |
| | GGAACCCACCTACTTTAAGGTAGGTATTGCAACATGAATACATAGTGTTGCAAGGCGAACGAG |
| | GGGAAGTGAAACATCTCAGTACCCTTAGGAAAAGAAATCAATTGAGATTCCCTCAGTAGCGGC |
| | GAGCGAACGGGGATCAGCCCATTAAGTTATGTGTGTTTTAGTGGAACGCTCTGGGAAGTGCGA |
| | ACGTAGAGGGTGATATTCCCGTACACGAAAGGGCACACATAATGATGACGAGTAGGGCGAGGC |
| | ACGTGAAACCTTGTCTGAATATGGGGGGACCATCCTCCAAGGCTAAATACTCCTGACTGACCG |
| | ATAGTGAACCAGTACCGTGAGGGAAAGGCGAAAAGAACCCCTGTGAGGGGAGTGAAATAGATC |
| | CTGAAACCGCATGCATACAAGCAGTGGGAGCACCTTCGTGGTGTGACTGCGTACCTTTTGTAT |
| | AATGGGTCAGCGACTTATATTCAGTAGCGAGGTTAACCGTATAGGGGAGCCGTAGAGAAATCG |
| | AGTCTTAATAGGGCGTTTAGTTGCTGGGTATAGACCCGAAACCAGGCGATCTATCCATGAGCA |
| | GGTTGAAGGTTGGGTAACACTAACTGGAGGACCGAACCCACTGTCGTTGAAAAGCCAGGGGAT |
| | GACTTGTGGATAGGGGTGAAAGGCTAATCAAGCCTGGTGATAGCTGGTTCTCCCCGAAAGCTA |
| | TTTAGGTAGCGCCTCGGACGAATACCATAGGGGGTAGAGCACTGTTTCGGCTAGGGGGTCATC |
| | CCGACTTACCAAACCGATGCAAACTCCGAATACCTATGAGTACTATCCGGGAGACAGACTGCG |
| | GGTGCTAACGTCCGTAGTCAAGAGGAAAACAATCCAGACCGCCAGCTAAGGCCCCAAAATCAT |
| | AGTTAAGTGGGAAACGATGTGGGAAGGCATAGACAGCTAGGAGGTTGGCTTAGAAGCAGCCAC |
| | CCTTTAAAGAAAGCGTAATAGCTCACTAGTCGAGTCGGCCTGCGCGGAAGATGTAACGGGGCT |
| | AAAACTATGTGCCGAAGCTGCGGATTTGACATTAGTCAAGTGGTAGGGGAGCGTTCTGTAAGC |
| | CGATGAAGGTGTATTGAGAAGTATGCTGGAGGTATCAGAAGTGCGAATGCTGACGTGAGTAAC |
| | GACAAAACGGGTGAAAAACCCGTTCGCCGAAAGACCAAGGGTTCCAGTCCAACGTTAATCGGG |
| | GCTGGGTGAGTCGACCCCTAAGGCGAGGCCGAAAGGCGTAGTCGATGGGAAATTGGTTAATAT |
| | TCCAATACTTCTGTGTAATGCGATGAGAGGACGGAGAAGGTTAAGTCAGCCTGGCGTTGGTTG |
| | TCCAGGTGGAAGGATGTAGGTATGTATCTTAGGCAAATCCGGGGTACTCTATACTGAGATCCG |
| | ATAGCAAGCTGTACTTGTACAGCGAAGTGGCTGATACCATGCTTCCAGGAAAAGTCTCTAAGC |
| | TTCAGTTACACAGGAATCGTACCCGAAACCGACACAGGTGGTCAGGTCGAGTAGACCAAGGCG |
| | CTTGAGAGAACTCTGCTGAAGGAACTAGGCAAAATGGTACCGTAACTTCGGGAGAAGGTACGC |
| | TGTTGTTGGTGATGGAACTTGCTTCCTGAGCTGATGACAGCCGCAGAAACCAGGCCGCTGCAA |
| | CTGTTTATTAAAAACATAGCACTCTGCAAACACGAAAGTGGACGTATAGGGTGTGATGCCTGC |
| | CCGGTGCTGGAAGGTTAATTGATGGGGTTAGCGTAAGCGAAGCTCTTGATCGAAGCCCCAGTA |
| | AACGGCGGCCGTAACTATAACGGTCCTAAGGTAGCGAAATTCCTTGTCGGGTAAGTTCCGgCC |
| | TGCACGAATGGCATAATGATGGCGGCGCTGTCTCCAGCAGAGGCTCAGTGAAATCGAAATCGC |
| | TGTGAAGATGCAGTGTACCCGCGGCTAGACGGAAAGACCCCGTGAACCTTTACTGCAGCTTGA |
| | CACTGAACTTTGACCTTACTTGTGTAGGATAGGTGGGAGGCTTTGAAGTTGGAACGCTAGTTC |
| | CAATGGAGCCGTCCTTGAAATACCACCCTGGTAATGTTGAGGTTCTAACTCTGTCCCGTTATC |
| | CGGGACGAGGACCGTGTCTGGTGGGTAGTTTGACTGGGGCGGTCTCCTCCTAAAGAGTAACGG |
| | AGGAGTACGAAGGTGCGCTCAGCGTGGTCGGAAATCACGCATAGAGTATAAAGGCAAAAGCGC |
| | GCTTAACTGCGAGACCCACAAGTCGAGCAGGTACGAAAGTAGGTCTTAGTGATCCGGTGGTTC |
| | TGTATGGAAGGGCCATCGCTCAACGGATAAAAGGTACTCTGGGGATAACAGGCTGATACCGCC |
| | CAAGAGTTCATATCGACGGCGGTGTTTGGCACCTCGATGTCGGCTCATCTCATCCTGGGGCTG |
| | AAGCAGGTCCCAAGGGTATGGCTGTTCGCCATTTAAAGAGGTACGCGAGCTGGGTTTAGAACG |
| | TCGTGAGACAGTTCGGTCCCTATCTACCGTGGGCGCTGGAAATTTGAGAGGATCTGCTCCTAG |
| | TACGAGAGGACCAGAGTGGACGAACCTCTGGTGTACCGGTTGTGACGCCAGTCGCATCGCCGG |
| | GTAGCTATGTTCGGAAGGGATAACCGCTGAAAGCATCTAAGCGGGAAGCCTACCTCAAGATAA |
| | GATTTCCCTAGGAATTTATTCCTCTAAAGAGCCGTTCGAGACTAGGACGTTGATAGGTTGGAT |
| | GTGGAAGCATAGTGATATGTGAAGCTGACCAATACTAATTGCTCGTGAGGCTTGACTAT-3′ |
| | (SEQ ID NO: 420) |
| V. cholerae 23S | 5′-GGTTAAGTGACTAAGCGTACACGGTGGATGCCTGGGCAGTCAGAGGCGATGAAGGACGTA |
| | CTAACTTGCGATAAGCGCAGATAAGGCAGTAAGAGCCGTTTGAGTCTGCGATTTCCGAATGGG |
| | GAAACCCAACTGCATAAGCAGTTACTGTTAACTGAATACATAGGTTAACAGAGCAAACCGGGG |
| | GAACTGAAACATCTAAGTACCCCGAGGAGAAGAAATCAACCGAGATTCCGGTAGTAGCGGCGA |
| | GCGAACCTGGATTAGCCCTTAAGCACTCGGTGAAGTAGGTGAACAAGCTGGAAAGCTTGGCGA |
| | TACAGGGTGATAGCCCCGTAACCGACGCTTCATCGAGCGTGAAATCGAGTAGGGCGGGACACG |
| | TGATATCCTGTCTGAATATGGGGGGACCATCCTCCAAGGCTAAATACTCCTGACTGACCGATA |
| | GTGAACCAGTACCGTGAGGGAAAGGCGAAAAGAACCCCTGTGAGGGGAGTGAAATAGAACCTG |
| | AAACCGTGTACGTACAAGCAGTAGGAGCACCTTCGTGGTGTGACTGCGTACCTTTTGTATAAT |
| | GGGTCAGCGACTTATATTCAGTGGCAAGGTTAACCGTATAGGGGAGCCGTAGCGAAAGCGAGT |
| | CTTAATTGGGCGCTCAGTCTCTGGATATAGACCCGAAACCGGGTGATCTAGCCATGGGCAGGT |
| | TGAAGGTTGAGTAACATCAACTGGAGGACCGAACCGACTAATGTTGAAAAATTAGCGGATGAC |
| | TTGTGGCTAGGGGTGAAAGGCCAATCAAACTCGGAGATAGCTGGTTCTCCCCGAAAGCTATTT |
| | AGGTAGCGCCTCGGACGAATACTACTGGGGGTAGAGCACTGTTAAGGCTAGGGGGTCATCCCG |
| | ACTTACCAACCCTTTGCAAACTCCGAATACCAGTAAGTACTATCCGGGAGACACACGGCGGGT |
| | GCTAACGTCCGTCGTGGAGAGGGAAACAACCCAGACCGCCAGCTAAGGTCCCAAAGTATTGCT |
| | AAGTGGGAAACGATGTGGGAAGGCTCAGACAGCTAGGATGTTGGCTTAGAAGCAGCCATCATT |
| | TAAAGAAAGCGTAATAGCTCACTAGTCGAGTCGGCCTGCGCGGAAGATGTAACGGGGCTAAGC |
| | AATACACCGAAGCTGCGGCAATGTCTTTTAGATATTGGGTAGGGGAGCGTTCTGTAAGCCGTT |
| | GAAGGTGAATCGTAAGGTTTGCTGGAGGTATCAGAAGTGCGAATGCTGACATGAGTAACGACA |
| | AAGGGGGTGAAAAACCTCCTCGCCGGAAGACCAAGGGTTCCTGTCCAACGTTAATCGGGGCAG |
| | GGTGAGTCGACCCCTAAGGTGAGGCCGAAAGGCGTAATCGATGGGAAACGGGTTAATATTCCC |
| | GTACTTCTGACTATTGCGATGGGGGGACGGAGAAGGCTAGGTGGGCCAGGCGACGGTTGTCCT |
| | GGTTCAAGTGCGTAGGCTTGAGAGTTAGGTAAATCCGGCTCTCTTTAAGGCTGAGACACGACG |
| | TCGAGCTGCTACGGCAGTGAAGTCATTGATGCCATGCTTCCAGGAAAAGCCTCTAAGCTTCAG |
| | ATAGTCAGGAATCGTACCCCAAACCGACACAGGTGGTCGGGTAGAGAATACCAAGGCGCTTGA |
| | GAGAACTCGGGTGAAGGAACTAGGCAAAATGGTACCGTAACTTCGGGAGAAGGTACGCTCTTG |
| | ATGGTGAAGTCCCTCGCGGATGGAGCTGACGAGAGTCGCAGATACCAGGTGGCTGCAACTGTT |
| | TATTAAAAACACAGCACTGTGCAAAATCGCAAGATGACGTATACGGTGTGACGCCTGCCCGGT |
| | GCCGGAAGGTTAATTGATGGGGTTAGCGCAAGCGAAGCTCTTGATCGAAGCCCCGGTAAACGG |
| | CGGCCGTAACTATAACGGTCCTAAGGTAGCGAAATTCCTTGTCGGGTAAGTTCCGACCTGCAC |
| | GAATGGCGTAATGATGGCCACGCTGTCTCCACCCGAGACTCAGTGAAATTGAAATCGCTGTGA |
| | AGATGCAGTGTACCCGCGGCTAGACGGAAAGACCCCGTGAACCTTTACTACAGCTTGGCACTG |
| | AACATTGAACCTACATGTGTAGGATAGGTGGGAGTCTATGAAGACGTGACGCCAGTTGCGTTG |
| | GAGCCGTCCTTGAAATACCACCCTTGTATGTTTGATGTTCTAACGTTGGCCCCTAATCGGGGT |
| | TGCGGACAGTGCCTGGTGGGTAGTTTGACTGGGGCGGTCTCCTCCCAAAGAGTAACGGAGGAG |
| | CACGAAGGTGGGCTAATCACGGTTGGACATCGTGAGGTTAGTGCAATGGCATAAGCCCGCTTA |
| | ACTGCGAGAATGACGGTTCGAGCAGGTGCGAAAGCAGGTCATAGTGATCCGGTGGTTCTGTAT |
| | GGAAGGGCCATCGCTCAACGGATAAAAGGTACTCCGGGGATAACAGGCTGATACCGCCCAAGA |
| | GTTCATATCGACGGCGGTGTTTGGCACCTCGATGTCGGCTCATCACATCCTGGGGCTGAAGTC |
| | GGTCCCAAGGGTATGGCTGTTCGCCATTTAAAGTGGTACGCGAGCTGGGTTTAGAACGTCGTG |
| | AGACAGTTCGGTCCCTATCTGCCGTGGGCGTTGGAAGATTGAAGGGGGCTGCTCCTAGTACGA |
| | GAGGACCGGAGTGGACGAACCTCTGGTGTTCGGGTTGTGTCGCCAGACGCATTGCCCGGTAGC |
| | TAAGTTCGGAATTGATAAGCGCTGAAAGCATCTAAGCGCGAAGCGAGCCCTGAGATGAGTCTT |
| | CCCTGACGGTTTAACCGTCCTAAAGGGTTGTTCGAGACTAGAACGTTGATAGGCAGGGTGTGT |
| | AAGCGTTGTGAGGCGTTGAGCTAACCTGTACTAATTGCCCGTGAGGCTTAACCAT-3′ |
| | (SEQ ID NO: 421) |
| P. aeruginosa 23S | 5′-GGTCAAGTGAAGAAGCGCATACGGTGGATGCCTTGGCAGTCAGAGGCGATGAAAGACGTG |
| | GTAGCCTGCGAAAAGCTTCGGGGAGTCGGCAAACAGACTTTGATCCGGAGATCTCTGAATGGG |
| | GGAACCCACCTAGGATAACCTAGGTATCTTGTACTGAATCCATAGGTGCAAGAGGCGAACCAG |
| | GGGAACTGAAACATCTAAGTACCCTGAGGAAAAGAAATCAACCGAGATTCCCTTAGTAGTGGC |
| | GAGCGAACGGGGATTAGCCCTTAAGCTTCATTGATTTTAGCGGAACGCTCTGGAAAGTGCGGC |
| | CATAGTGGGTGATAGCCCCGTACGCGAAAGGATCTTTGAAGTGAAATCGAGTAGGACGGAGCA |
| | CGAGAAACTTTGTCTGAACATGGGGGGACCATCCTCCAAGGCTAAATACTACTGACTGACCGA |
| | TAGTGAACCAGTACCGTGAGGGAAAGGCGAAAAGAACCCCGGAGAGGGGAGTGAAATAGAACC |
| | TGAAACCGTATGCGTACAAGCAGTGGGAGCCTACTTGTTAGGTGACTGCGTACCTTTTGTATA |
| | ATGGGTCAGCGACTTATATTCAGTGGCAAGCTTAACCGTATAGGGTAGGCGTAGCGAAAGCGA |
| | GTCTTAATAGGGCGTTTAGTCGCTGGGTATAGACCCGAAACCGGGCGATCTATCCATGAGCAG |
| | GTTGAAGGTTAGGTAACACTGACTGGAGGACCGAACCCACTCCCGTTGAAAAGGTAGGGGATG |
| | ACTTGTGGATCGGAGTGAAAGGCTAATCAAGCTCGGAGATAGCTGGTTCTCCTCGAAAGCTAT |
| | TTAGGTAGCGCCTCATGTATCACTCTGGGGGGTAGAGCACTGTTTCGGCTAGGGGGTCATCCC |
| | GACTTACCAAACCGATGCAAACTCCGAATACCCAGAAGTGCCGAGCATGGGAGACACACGGCG |
| | GGTGCTAACGTCCGTCGTGAAAAGGGAAACAACCCAGACCGCCAGCTAAGGTCCCAAAGTTGT |
| | GGTTAAGTGGTAAACGATGTGGGAAGGCTTAGACAGCTAGGAGGTTGGCTTAGAAGCAGCCAC |
| | CCTTTAAAGAAAGCGTAATAGCTCACTAGTCGAGTCGGCCTGCGCGGAAGATGTAACGGGGCT |
| | CAAACCACACACCGAAGCTGCGGGTGTCACGTAAGTGACGCGGTAGAGGAGCGTTCTGTAAGC |
| | CTGTGAAGGTGAGTTGAGAAGCTTGCTGGAGGTATCAGAAGTGCGAATGCTGACATGAGTAAC |
| | GACAATGGGTGTGAAAAACACCCACGCCGAAAGACCAAGGGTTCCTGCGCAACGTTAATCGAC |
| | GCAGGGTTAGTCGGTTCCTAAGGCGAGGCTGAAAAGCGTAGTCGATGGGAAACAGGTTAATAT |
| | TCCTGTACTTCTGGTTACTGCGATGGAGGGACGGAGAAGGCTAGGCCAGCTTGGCGTTGGTTG |
| | TCCAAGTTTAAGGTGGTAGGCTGAAATCTTAGGTAAATCCGGGGTTTCAAGGCCGAGAGCTGA |
| | TGACGAGTCGTCTTTTAGATGACGAAGTGGTTGATGCCATGCTTCCAAGAAAAGCTTCTAAGC |
| | TTCAGGTAACCAGGAACCGTACCCCAAACCGACACAGGTGGTCGGGTAGAGAATACCAAGGCG |
| | CTTGAGAGAACTCGGGTGAAGGAACTAGGCAAAATGGCACCGTAACTTCGGGAGAAGGTGCGC |
| | CGGCTAGGGTGAAGGATTTACTCCGTAAGCTCTGGCTGGTCGAAGATACCAGGCCGCTGCGAC |
| | TGTTTATTAAAAACACAGCACTCTGCAAACACGAAAGTGGACGTATAGGGTGTGACGCCTGCC |
| | CGGTGCCGGAAGGTTAATTGATGGGGTTAGCGCAAGCGAAGCTCTTGATCGAAGCCCCGGTAA |
| | ACGGCGGCCGTAACTATAACGGTCCTAAGGTAGCGAAATTCCTTGTCGGGTAAGTTCCGACCT |
| | GCACGAATGGCGTAACGATGGCGGCGCTGTCTCCACCCGAGACTCAGTGAAATTGAAATCGCT |
| | GTGAAGATGCAGTGTATCCGCGGCTAGACGGAAAGACCCCGTGAACCTTTACTGTAGCTTTGC |
| | ACTGGACTTTGAGCCTGCTTGTGTAGGATAGGTGGGAGGCTTTGAAGCGTGGACGCCAGTTCG |
| | CGTGGAGCCATCCTTGAAATACCACCCTGGCATGCTTGAGGTTCTAACTCTGGTCCGTAATCC |
| | GGATCGAGGACAGTGTATGGTGGGCAGTTTGACTGGGGCGGTCTCCTCCTAAAGAGTAACGGA |
| | GGAGTACGAAGGTGCGCTCAGACCGGTCGGAAATCGGTCGCAGAGTATAAAGGCAAAAGCGCG |
| | CTTGACTGCGAGACAGACACGTCGAGCAGGTACGAAAGTAGGTCTTAGTGATCCGGTGGTTCT |
| | GTATGGAAGGGCCATCGCTCAACGGATAAAAGGTACTCCGGGGATAACAGGCTGATACCGCCC |
| | AAGAGTTCATATCGACGGCGGTGTTTGGCACCTCGATGTCGGCTCATCACATCCTGGGGCTGA |
| | AGCCGGTCCCAAGGGTATGGCTGTTCGCCATTTAAAGTGGTACGCGAGCTGGGTTTAGAACGT |
| | CGTGAGACAGTTCGGTCCCTATCTGCCGTGGACGTTTGAGATTTGAGAGGGGCTGCTCCTAGT |
| | ACGAGAGGACCGGAGTGGACGAACCTCTGGTGTTCCGGTTGTCACGCCAGTGGCATTGCCGGG |
| | TAGCTATGTTCGGAAAAGATAACCGCTGAAAGCATCTAAGCGGGAAACTTGCCTCAAGATGAG |
| | ATCTCACTGGGAACTTGATTCCCCTGAAGGGCCGTCGAAGACTACGACGTTGATAGGCTGGGT |
| | GTGTAAGCGTTGTGAGGCGTTGAGCTAACCAGTACTAATTGCCCGTGAGGCTTGACCAT-3′ |
| | (SEQ ID NO: 422) |
| S. marcescens 23S | 5′-GGTTAAGTGACTAAGCGTACACGGTGGATGCCTAGGCAGTCAGAGGCGATGAAGGGCGTG |
| | CTAATCTGCGAAAAGCGTCGGTAAGGTGATATGAACCGTTATAACCGGCGATACCCGAATGGG |
| | GAAACCCAGTGTGTTTCGACACACTATCATGTCATGAATACATAGTGGCATGAGGCGAACCGG |
| | GGGAACTGAAACATCTAAGTACCCCGAGGAAAAGAAATCAACCGAGATTCCCCCAGTAGCGGC |
| | GAGCGAACGGGGAGGAGCCCAGAACCTGAATCGGCTTGTGTGTTAGTGGAAGCGTCTGGAAAG |
| | TCGCGCAGCAAAGGGTGATAGCCCCGTACACTAAAATGCACAGGTCGTGAGTTCGATGAGTAG |
| | GGCGGGACACGTGACATCCTGTCTGAATATGGGGGGACCATCCTCCAAGGCTAAATACTCCTG |
| | ACTGACCGATAGTGAACCAGTACCGTGAGGGAAAGGCGAAAAGAACCCCGGCGAGGGGAGTGA |
| | AATAGAACCTGAAACCGTGTACGTACAAGCAGTGGGAGCACCTTCGTGGTGTGACTGCGTACC |
| | TTTTGTATAATGGGTCAGCGACTTATATTTTGTAGCAAGGTTAACCGTATAGGGGAGCCGTAG |
| | GGAAACCGAGTCTTAACTGGGCGAATAGTTGCAAGGTATAGACCCGAAACCCGGTGATCTAGC |
| | CATGGGCAGGTTGAAGGTTGGGTAACACTAACTGGAGGACCGAACCGACTAATGTTGAAAAAT |
| | TAGCGGATGACTTGTGGCTGGGGGTGAAAGGCCAATCAAACCGGGAGATAGCTGGTTCTCCCC |
| | GAAAGCTATTTAGGTAGCGCCTCGTGAACTCATCTTCGGGGGTAGAGCACTGTTTCGGCTAGG |
| | GGGCCATCCCGGCTTACCAAACCGATGCAAACTCCGAATACCGAAGAATGTTATCACGGGAGA |
| | CACACGGCGGGTGCTAACGTCCGTCGTGAAGAGGGAAACAACCCAGACCGCCAGCTAAGGTCC |
| | CAAAGTCATGGTTAAGTGGGAAACGATGTGGGAAGGCATAGACAGCCAGGATGTTGGCTTAGA |
| | AGCAGCCATCATTTAAAGAAAGCGTAATAGCTCACTGGTCGAGTCGGCCTGCGCGGAAGATGT |
| | AACGGGGCTAAACCATGCACCGAAGCTGCGGCAGCGACGCTTAGGCGTTGTTGGGTAGGGGAG |
| | CGTTCTGTAAGCCGTTGAAGGTGGCCTGTGAGGGTTGCTGGAGGTATCAGAAGTGCGAATGCT |
| | GACATAAGTAACGATAAAGCGGGTGAAAAGCCCGCTCGCCGGAAGACCAAGGGTTCCTGTCCA |
| | ACGTTAATCGGGGCAGGGTGAGTCGACCCCTAAGGCGAGGCTGAAAAGCGTAGTCGATGGGAA |
| | ACAGGTTAATATTCCTGTACTTGGTGTTACTGCGAAGGGGGGACGGAGAAGGCTAGGCTAGCC |
| | GGGCGACGGTTGTCCCGGTTTAAGCGTGTAGGGGGTGTGACCTGGTAAATCCGGTTGCATATT |
| | AACCCTGAGGCGTGATGACGATGCACTACGGTGCAGAAGTAGTTGATGCCCTGCTTCCAGGAA |
| | AATCCTCTAAGCATCAGGTAACATTAAATCGTACCCCAAACCGACACAGGTGGTCAGGTAGAG |
| | AATACCAAGGCGCTTGAGAGAACTCGGGTGAAGGAACTAGGCAAAATGGTGCCGTAACTTCGG |
| | GAGAAGGCACGCTGGCATGTAGGTGAAGTCCCTCGCGGATGGAGCTGAAGCCAGTCGAAGATA |
| | CCAGCTGGCTGCAACTGTTTAATAAAAACACAGCACTGTGCAAACACGAAAGTGGACGTATAC |
| | GGTGTGACGCCTGCCCGGTGCTGGAAGGTTAATTGATGGGGTCAGCCGCAAGGCGAAGCTCTT |
| | GATCGAAGCCCCAGTAAACGGCGGCCGTAACTATAACGGTCCTAAGGTAGCGAAATTCCTTGT |
| | CGGGTAAGTTCCGACCTGCACGAATGGCGTAATGATGGCCAGGCTGTCTCCACCCGAGACTCA |
| | GTGAAATTGAACTCGCTGTGAAGATGCAGTGTACCCGCGGCAAGACGGAAAGACCCCGTGAAC |
| | CTTTACTATAGCTTGACACTGAACATTGAGCCTTGATGTGTAGGATAGGTGGGAGGCTTTGAA |
| | GCGTGGACGCCAGTCTGCGTGGAGCCATCCTTGAAATACCACCCTTTAATGTTTGATGTTCTA |
| | ACTCGGCCCCGTAATCCGGGGTGAGGACAGTGTCTGGTGGGTAGTTTGACTGGGGCGGTCTCC |
| | TCCCAAAGAGTAACGGAGGAGCACGAAGGTTAGCTAATCACGGTCGGACATCGTGAGGTTAGT |
| | GCAAAGGCATAAGCTAGCTTGACTGCGAGAGTGACGGCTCGAGCAGGTACGAAAGTAGGTCTT |
| | AGTGATCCGGTGGTTCTGAATGGAAGGGCCATCGCTCAACGGATAAAAGGTACTCCGGGGATA |
| | ACAGGCTGATACCGCCCAAGAGTTCATATCGACGGCGGTGTTTGGCACCTCGATGTCGGCTCA |
| | TCACATCCTGGGGCTGAAGTAGGTCCCAAGGGTATGGCTGTTCGCCATTTAAAGTGGTACGCG |
| | AGCTGGGTTTAGAACGTCGTGAGACAGTTCGGTCCCTATCTGCCGTGGGCGTTGGAAGATTGA |
| | GAGGGGTTGCTCCTAGTACGAGAGGACCGGAGTGAACGCACCACTGGTGTTCGGGTTGTCATG |
| | CCAATGGCATTGCCCGGTAGCTAAGTGCGGAAAAGATAAGCGCTGAAAGCATCTAAGCGCGAA |
| | ACTTGCCTCAAGATGAGTCTTCCCTGGGCCTTTAAGGTCCCTGAAGGAACGTTTAAGACTAAG |
| | ACGTTGATAGGCTGGGTGTGTAAGTGCAGCGATGCATTGAGCTAACCAGTACTAATGATCCGT |
| | GAGGCTTAACCTT-3′ (SEQ ID NO: 423) |
| S. enterica 23S | 5′-GGTTAAGCGACTAAGCGTACACGGTGGATGCCCTGGCAGTCAGAGGCGATGAAGGGCGTG |
| | CTAATCTGCGATAAGCGCCGGTAAGGTGATATGAACCGTTATAACCGGCGATACCCGAATGGG |
| | GAAACCCAGTGTGATTCGTCACACTATCATTAACTGAATCCATAGGTTAATGAGGCGAACCGG |
| | GGGAACTGAAACATCTAAGTACCCCGAGGAAAAGAAATCAACCGAGATTCCCCCAGTAGCGGC |
| | GAGCGAACGGGGAGGAGCCCAGAGCCTGAATCAGCATGTGTGTTAGTGGAAGCGTCTGGAAAG |
| | GTGCGCGATACAGGGTGACAGCCCCGTACACAAAAGCGCATGTGCTGTGAGCTCGATGAGTAG |
| | GGCGGGACACGTGGTATCCTGTCTGAATATGGGGGGACCATCCTCCAAGGCTAAATACTCCTG |
| | ACTGACCGATAGTGAACCAGTACCGTGAGGGAAAGGCGAAAAGAACCCCGGCGAGGGGAGTGA |
| | AAAAGAACCTGAAACCGTGTACGTACAAGCAGTGGGAGCACAGGTTTACCTGTGTGACTGCGT |
| | ACCTTTTGTATAATGGGTCAGCGACTTATATTCTGTAGCAAGGTTAACCGTATAGGGGAGCCG |
| | TAGGGAAACCGAGTCTTAACCGGGCGTTAAGTTGCAGGGTATAGACCCGAAACCCGGTGATCT |
| | AGCCATGGGCAGGTTGAAGGTTGGGTAACACTAACTGGAGGACCGAACCGACTAATGTTGAAA |
| | AATTAGCGGATGACCTGTGGCTGGGGGTGAAAGGCCAATCAAACCGGGAGATAGCTGGTTCTC |
| | CCCGAAAGCTATTTAGGTAGCGCCTCGTGAATTCATCTCCGGGGGTAGAGCACTGTTTCGGCT |
| | AGGGGGCCATCCCGGCTTACCAACCCGATGCAAACTGCGAATACCGGAGAATGTTATCACGGG |
| | AGACACACGGCGGGTGCTAACGTCCGTCGTGAAGAGGGAAACAACCCAGACCGCCAGCTAAGG |
| | TCCCAAAGTCATGGTTAAGTGGGAAACGATGTGGGAAGGCCCAGACAGCCAGGATGTTGGCTT |
| | AGAAGCAGCCATCATTTAAAGAAAGCGTAATAGCTCACTGGTCGAGTCGGCCTGCGCGGAAGA |
| | TGTAACGGGGCTAAACCATGCACCGAAGCTGCGGCAGCGAACGTATCACCCAAGACAACTTTA |
| | CGGAGTTGACGATTGACGGAGCGAAGCGACGTCAAAGCGTTCATTAAAGTCGAGTTGGCTTAG |
| | GGATACGTTCGTTGGGTAGGGGAGCGTTCTGTAAGCCTGTGAAGGTGTGTCGTGAGGCATGCT |
| | GGAGGTATCAGAAGTGCGAATGCTGACATAAGTAACGATAAAGCGGGTGAAAAGCCCGCTCGC |
| | CGGAAGACCAAGGGTTCCTGTCCAACGTTAATCGGGGCAGGGTGAGTCGACCCCTAAGGCGAG |
| | GCCGAAAGGCGTAGTCGATGGGAAACGGGTTAATATTCCCGTACTTGGTGTTACTGCGAAGGG |
| | GGGACGGAGAAGGCTATGTTGGCCGGGCGACGGTTGTCCCGGTTTAAGCGTGTAGGTGTGTGT |
| | TCCAGGTAAATCCGGTTCACTTTAACACTGAGGCGTGACGACGAGGCACTACGGTGCTGAAGC |
| | AACAAATGCCCTGCTTCCAGGAAAAGCCTCTAAGCATCAGGTAACACGAAATCGTACCCCAAA |
| | CCGACACAGGTGGTCAGGTAGAGAATACCAAGGCGCTTGAGAGAACTCGGGTGAAGGAACTAG |
| | GCAAAATGGTGCCGTAACTTCGGGAGAAGGCACGCTGACACGTAGGTGAAGTGATTTACTCAT |
| | GGAGCTGAAGTCAGTCGAAGATACCAGCTGGCTGCAACTGTTTATTAAAAACACAGCACTGTG |
| | CAAACACGAAAGTGGACGTATACGGTGTGACGCCTGCCCGGTGCCGGAAGGTTAATTGATGGG |
| | GTCAGCGCAAGCGAAGCTCCTGATCGAAGCCCCGGTAAACGGCGGCCGTAACTATAACGGTCC |
| | TAAGGTAGCGAAATTCCTTGTCGGGTAAGTTCCGACCTGCACGAATGGCGTAATGATGGCCAG |
| | GCTGTCTCCACCCGAGACTCAGTGAAATTGAACTCGCTGTGAAGATGCAGTGTACCCGCGGCA |
| | AGACGGAAAGACCCCGTGAACCTTTACTATAGCTTGACACTGAACATTGAGCCTTGATGTGTA |
| | GGATAGGTGGGAGGCTTTGAAGTGTGGACGCCAGTCTGCATGGAGCCGACCTTGAAATACCAC |
| | CCTTTAATGTTTGATGTTCTAACGTGGACCCGTAATCCGGGTTGCGGACAGTGTCTGGTGGGT |
| | AGTTTGACTGGGGCGGTCTCCTCCTAAAGAGTAACGGAGGAGCACGAAGGTTGGCTAATCCTG |
| | GTCGGACATCAGGAGGTTAGTGCAATGGCATAAGCCAGCTTGACTGCGAGCGTGACGGCGCGA |
| | GCAGGTGCGAAAGCAGGTCATAGTGATCCGGTGGTTCTGAATGGAAGGGCCATCGCTCAACGG |
| | ATAAAAGGTACTCCGGGGATAACAGGCTGATACCGCCCAAGAGTTCATATCGACGGCGGTGTT |
| | TGGCACCTCGATGTCGGCTCATCACATCCTGGGGCTGAAGTAGGTCCCAAGGGTATGGCTGTT |
| | CGCCATTTAAAGTGGTACGCGAGCTGGGTTTAGAACGTCGTGAGACAGTTCGGTCCCTATCTG |
| | CCGTGGGCGCTGGAGAACTGAGGGGGGCTGCTCCTAGTACGAGAGGACCGGAGTGGACGCATC |
| | ACTGGTGTTCGGGTTGTCATGCCAATGGCACTGCCCGGTAGCTAAATGCGGAAGAGATAAGTG |
| | CTGAAAGCATCTAAGCACGAAACTTGCCCCGAGATGAGTTCTCCCTGAGACTTAGAGTCTCCT |
| | GAAGGAACGTTGAAGACGACGACGTTGATAGGCCGGGTGTGTAAGCGCAGCGATGCGTTGAGC |
| | TAACCGGTACTAATGAACCGTGAGGCTTAACCTT-3′ (SEQ ID NO: 424) |
| N. gonorrhoeae 23S | 5′-AGTCAAGTGAATAAGTGCATCAGGCGGATGCCTTGGCGATGATAGGCGACGAAGGACGTG |
| | TAAGCCTGCGAAAAGCGCGGGGGAGCTGGCAATAAAGCtATGATtCCGCGaTGTCCGAATGGG |
| | GAAACCCACTGCATTCTGTGCAGTATCCTAAGTTGAATACATAGGCTTAGAGAAGCGAACCCG |
| | GAGAACTGAaCCATCTAAGTACCCGGAGGAAAAGAAATCAACCGAGATTCCGCAAGTAGTGGC |
| | GAGCGAACGCGGAGGAGCCTGTACGTAATAACTGTCGAGaTAGAAGAACAAGCTGGGAAGCTT |
| | GACCATAGCGGGTGACAGTCCCGTATTCGAAATCTCAACAGCGGTACTAAGCGTACGAAAAGT |
| | AGGGCGGGACACGTGAAATCCTGTCTGAATATGGGGGGACCATCCTCCAAGGCTAAATACTCA |
| | TCATCGACCGATAGTGAACCAGTACCGTGAGGGAAAGGCGAAAAGAACCCCGGGAGGGGAGTG |
| | AAACAGAACCTGAAACCTGATGCATACAAACAGTGGGAGCGCCCTAGTGGTGTGACTGCGTAC |
| | CTTTTGTATAATGGGTCAACGACTTACATTCAGTAGCGAGCTTAACCGGATAGGGGAGGCGTA |
| | GGGAAACCGAGTCTTAATAGGGCGATGAGTTGCTGGGTGTAGACCCGAAACCGAGTGATCTAT |
| | CCATGGtCAGGTTGAAGGTGCCGTAACAGGTACTGGAGGACCGAACCCACGCATGTTGCAAAA |
| | TGCGGGGATGAGCTGTGGGTAGGGGTGAAAGGCTAAACAAACTCGGAGATAGCTGGTTCTCCC |
| | CGAAAACTATTTAGGTAGTGCCTCGAGCAAGACACTGATGGGGGTAAAGCACTGTTATGGCTA |
| | GGGGGTTATTGCAACTTACCAACCCATGGCAAACTCAGAATACCATCAAGTGGTTCCTCGGGA |
| | GACAGACAGCGGGTGCTAACGTCCGTTGTCAAGAGGGAAACAACCCAGACCGCCGGCTAAGGT |
| | CCCAAATGATAGATTAAGTGGTAAACGAAGTGGGAAGGCACAGACAGCCAGGATGTTGGCTTA |
| | GAAGCAGCCATCATTTAAAGAAAGCGTAATAGCTCACTGGTCGAGTCGTCCTGCGCGGAAGAT |
| | GTAACGGGGCTCAAATCTATAACCGAAGCTGCGGATGCCGGTTTACCGGCATGGTAGGGGAGC |
| | GTTCTGTAGGCTGATGAAGGTGCATTGTAAAGTGTGCTGGAGGTATCAGAAGTGCGAATGTTG |
| | ACATGAGTAGCGATAAAGCGGGTGAAAAGCCCGCTCGCCGAAAGCCCAAGGTTTCCTACGCAA |
| | CGTTCATCGGCGTAGGGTaAGTCGGCCCCTAAGGCGAGGCAGAAATGCGTAGTCGATGGGAAA |
| | CAGGTTAATATTCCTGTACTTGATTCAAATGCGATGTGGGGACGGAGAAGGTTAGGTTGGCAA |
| | GCTGTTGGAATAGCTTGTTTAAGCCGGTAGGTGGAAGACTTAGGCAAATCCGGGTTTTCTTAA |
| | CACCGAGAAGTGATGACGAGTGTCTACGGACACGAAGCAACCGATACCACGCTTCCAGGAAAA |
| | GCCACTAAGCTTCAGTTTGAATCGAACCGTACCGCAAACCGACACAGGTGGGCAGGATGAGAA |
| | TTCTAAGGCGCTTGAGAGAACTCGGGAGAAGGAACTCGGCAAATTGATACCGTAACTTCGGGA |
| | GAAGGTATGCCCTCTAAGGTTAAGGACTTGCTCCGTAAGCCCCGGAGGGTCGCAGAGAATAGG |
| | TGGCTGCGACTGTTTATTAAAAACACAGCACTCTGCCAACACGAAAGTGGACGTATAGGGTGT |
| | GACGCCTGCCCGGTGCCGGAAGGTTAATTGAAGATGTGCAAGCATCGGATCGAAGCCCCGGTA |
| | AACGGCGGCCGTAACTATAACGGTCCTAAGGTAGCGAAATTCCTTGTCGGGTAAGTTCCGACC |
| | CGCACGAATGGCGTAACGATGGCCACACTGTCTCCTCCCGAGACTCAGCGAAGTTGAAGTGGT |
| | TGTGAAGATGCAATCTACCCGCTGCTAGACGGAAAGACCCCGTGAACCTTTACTGTAGCTTTG |
| | CATTGGACTTTGAAGTCACTTGTGTAGGATAGGTGGGAGGCTTGGAAGCAGAGACGCCAGTCT |
| | CTGTGGAGTCGTCCTTGAAATACCACCCTGGTGTCTTTGAGGTTCTAACCCAGACCCGTCATC |
| | CGGGTCGGGGACCGTGCATGGTAGGCAGTTTGACTGGGGCGGTCTCCTCCCAAAGCGTAACGG |
| | AGGAGTTCGAAGGTTACCTAGGTCCGGTCGGAAATCGGACTGATAGTGCAATGGCAAAAGGTA |
| | GCTTAACTGCGAGACCGACAAGTCGGGCAGGTGCGAAAGCAGGACATAGTGATCCGGTGGTTC |
| | TGTATGGAAGGGCCATCGCTCAACGGATAAAAGGTACTCCGGGGATAACAGGCTGATTCCGCC |
| | CAAGAGTTCATATCGACGGCGGAGTTTGGCACCTCGATGTCGGCTCATCACATCCTGGGGCTG |
| | TAGTCGGTCCCAAGGGTATGGCTGTTCGCCATTTAAAGTGGTACGTGAGCTGGGTTTAAAACG |
| | TCGTGAGACAGTTTGGTCCCTATCTGCAGTGGGCGTTGGAAGTTTGACGGGGGCTGCTCCTAG |
| | TACGAGAGGACCGGAGTGGACGAACCTCTGGTGTACCGGTTGTAACGCCAGTTGCATAGCCGG |
| | GTAGCTAAGTTCGGAAGAGATAAGCGCTGAAAGCATCTAAGCGCGAAACTCGCCTGAAGATGA |
| | GACTTCCCTTGCGGTTTAACCGCACTAAAGGGTCGTTCGAGACCAGGACGTTGATAGGTGGGG |
| | TGTGGAAGCGCGGTAACGCGTGAAGCTAACCCATACTAATTGCCCGTGAGGCTTGACTCT-3′ |
| | (SEQ ID NO: 425) |
| A. baumannn 5S | 5′-GTGCTGGCGACCATAGCAAGAGTGAACCACCTGATCCCTTCCCGAACTCAGAAGTGAAAC |
| | CTCTTAGCGCTGATGGTAGTGTGGGGTTACCCATGTGAGAGTAAGTCATCGCCAGCTCA-3′ |
| | (SEQ ID NO: 426) |
| V. cholerae 5S | 5′-TGCTTGGCGACCATAGCGTTTTGGACCCACCTGACTCCATCCCGAACTCAGAAGTGAAAC |
| | GAAACAGCGTCGATGGTAGTGTGGGGTCTCCCCATGTGAGAGTAGAACATCGCCAGGCTT-3′ |
| | (SEQ ID NO: 427) |
| P. aeruginosa 5S | 5′-TGCTTGACGATCATAGAGCGTTGGAACCACCTGATCCCTTCCCGAACTCAGAAGTGAAAC |
| | GACGCATCGCCGATGGTAGTGTGGGGTCTCCCCATGTGAGAGTAGGTCATCGTCAAGCTC-3′ |
| | (SEQ ID NO: 428) |
| S. marcescens 5S | 5′-GCCTGGCGGCAATAGCGCGGTGGTCCCACCTGACCCCATGCCGAACTCAGAAGTGAAACG |
| | CCGTAGCGCCGATGGTAGTGTGGGGTCTCCCCATGCGAGAGTAGGACACTGCCAGGCAT-3′ |
| | (SEQ ID NO: 429) |
| S. enterica 5S | 5′-TGCCTGGCGGCCGTAGCGCGGTGGTCCCACCTGACCCCATGCCGAACTCAGAAGTGAAAC |
| | GCCGTAGCGCCGATGGTAGTGTGGGGTCTCCCCATGCGAGAGTAGGGAACTGCCAGGCAT-3′ |
| | (SEQ ID NO: 430) |
| N. gonorrhoeae 5S | 5′-GTTTGGCGGCCATAGCGAGTTGGTCCCACGCCTTCCCATCCCGAACAGGACCGTGAAACG |
| | ACTCAGCGCCGATGATAGTGTGGTTCTTCCATGCGAAAGTAGGTCACTGCCAAACAC-3′ |
| | (SEQ ID NO: 431) |

TABLE 4
Exemplified and Expressly Contemplated Heterologous S20,
S16, S1 and S15 r-Protein Coding Sequences and Encoded r-Proteins

| Name | Sequence |
|---|---|
| N. gonorrhoeae | 5′-ATGGCAAACAGCGCACAAGCACGCAAACGTGCCCGCCAGTCCGTCAAACAACGCGCCCA |
| S20 RNA | CAACGCTAGCCTGCGTACCGCATTCCGCACCGCAGTGAAAAAAGTATTGAAAGCAGTCGAAG |
| | CAGGCGATAAAGCTGCCGCACGAGCGGTTTACCAAGAGTCCGTCAAAGTCATCGACCGCATC |
| | GCCGACAAAGGCGTGTTCCACAAAAACAAAGCGGCTCGCCACAAAAGCCGCCTGTCTGCAAA |
| | AGTAAAAGCACTGGCTTGA-3′ (SEQ ID NO: 432) |
| N. gonorrhoeae | N-MANSAQARKRARQSVKQRAHNASLRTAFRTAVKKVLKAVEAGDKAAARAVYQESVKVIDR |
| S20 Protein | IADKGVFHKNKAARHKSRLSAKVKALA-C (SEQ ID NO: 433) |
| N. gonorrhoeae | 5′-ATGGTAGTTATCCGTTTGGCACGCGGCGGCTCGAAACACCGCCCCTTCTACAACGTCAT |
| S20 Protein | CGTTACTGACTCACGCAGCCGCCGCGACGGCCGCTTCATCGAACGCGTAGGCTTCTACAACC |
| | CCGTAGCCAACGAAAAACAAGAGCGCGTCCGCCTCAATGCAGACCGCCTGAACCACTGGATT |
| | GCACAAGGCGCGCAAGTCAGCGACTCCGTTGCAAAACTGATTAAAGAACAAAAAGCCGTCTA |
| | A-3′ (SEQ ID NO: 434) |
| N. gonorrhoeae | N-MVVIRLARGGSKHRPFYNVIVTDSRSRRDGRFIERVGFYNPVANEKQERVRLNADRLNHW |
| S16 Protein | IAQGAQVSDSVAKLIKEQKAV-C (SEQ ID NO: 435) |
| N. gonorrhoeae | 5′-ATGTCTATGGAAAATTTTGCTCAGCTGTTGGAAGAAAGCTTTACCCTGCAAGAAATGAA |
| S1 RNA | CCCGGGTGAGGTGATTACCGCTGAAGTAGTGGCAATCGACCAAAACTTCGTTACCGTAAACG |
| | CAGGTCTGAAATCAGAATCCCTGATCGATGTAGCTGAATTCAAAAACGCTCAAGGCGAAATT |
| | GAAGTTAAAGTCGGCGACTTCGTTACCGTTACCATCGAATCCGTCGAAAACGGCTTCGGCGA |
| | AACCAAACTGTCCCGCGAAAAAGCCAAACGCGCAGCCGATTGGATCGCTTTGGAAGAAGCCA |
| | TGGAAAACGGCAACATCCTGTCCGGCATCATCAACGGTAAAGTCAAAGGCGGCCTGACCGTT |
| | ATGATCAGCAGCATCCGCGCATTCCTGCCGGGTTCTTTGGTCGACGTACGTCCCGTTAAAGA |
| | CACTTCCCATTTTGAAGGCAAAGAGATCGAATTCAAAGTGATCAAACTGGACAAAAAACGCA |
| | ACAACGTCGTTGTTTCCCGCCGCGCCGTTTTGGAAGCCACTTTGGGTGAAGAACGCAAAGCC |
| | CTGCTGGAAAACCTGCAAGAAGGCTCCGTCATCAAAGGCATCGTCAAAAATATCACCGACTA |
| | CGGCGCATTCGTTGACCTGGGCGGCATCGACGGCCTGCTGCACATCACCGATTTGGCATGGC |
| | GTCGCGTGAAACACCCGAGCGAAGTCTTGGAAGTCGGTCAGGAAGTTGAAGCCAAAGTATTG |
| | AAATTCGACCAAGAAAAACAACGTGTTTCCTTGGGTATGAAACAACTGGGCGAAGATCCTTG |
| | GAGCGGTCTGACCCGCCGTTATCCGCAAGCCACCCGCCTGTTCGGCAAAGTATCCAACCTGA |
| | CCGACTACGGCGCATTCGTCGAAATCGAACAAGGCATCGAAGGTTTGGTACACGTCTCCGAA |
| | ATGGACTGGACCAACAAAAACGTACACCCGAGCAAAGTCGTACAACTGGGTGACGAAGTCGA |
| | AGTCATGATTTTGGAAATCGACGAAGGCCGCCGCCGTATCTCTTTGGGTATGAAACAATGCC |
| | AAGCCAATCCTTGGGAAGAATTTGCCGCCAACCACAACAAAGGAGACAAAATCTCCGGTGCG |
| | GTTAAATCCATTACCGATTTCGGCGTATTCGTCGGCCTGCCCGGCGGCATCGACGGTCTGGT |
| | TCACCTGTCCGACCTGTCTTGGACCGAATCCGGCGAAGAAGCCGTACGCAAATACAAAAAAG |
| | GAGAAGAAGTCGAAGCCGTCGTATTGGCAATCGATGTGGAAAAAGAACGCATCTCCTTGGGT |
| | ATCAAACAACTGGAAGGCGATCCTTTCGGCAACTTCATCAGCGTGAACGACAAAGGTTCTTT |
| | GGTTAAAGGTTCCGTGAAATCTGTTGATGCCAAAGGCGCTGTTATCGCCCTGTCTGACGAAG |
| | TAGAAGGCTACCTGCCTGCTTCCGAATTTGCAGCCGACCGCGTTGAAGACTTGACCACCAAA |
| | CTGAAAGAAGGCGATGAAGTTGAAGCCGTCATCGTTACCGTTGACCGCAAAAACCGCAGCAT |
| | CAAACTTTCCGTTAAAGCCAAAGATGCCAAAGAAAGCCGCGAAGCACTGAACTCCGTCAATG |
| | CCGCCGCCAATGCGAATGCCGGTACCACCAGCTTGGGCGACCTGCTGAAAGCCAAACTCTCC |
| | GGCGAACAAGAATAA-3′ (SEQ ID NO: 436) |
| N. gonorrhoeae | N-MSMENFAQLLEESFTLQEMNPGEVITAEVVAIDQNFVTVNAGLKSESLIDVAEFKNAQGE |
| S1 Protein | IEVKVGDFVTVTIESVENGFGETKLSREKAKRAADWIALEEAMENGNILSGIINGKVKGGLT |
| | VMISSIRAFLPGSLVDVRPVKDTSHFEGKEIEFKVIKLDKKRNNVVVSRRAVLEATLGEERK |
| | ALLENLQEGSVIKGIVKNITDYGAFVDLGGIDGLLHITDLAWRRVKHPSEVLEVGQEVEAKV |
| | LKFDQEKQRVSLGMKQLGEDPWSGLTRRYPQATRLFGKVSNLTDYGAFVEIEQGIEGLVHVS |
| | EMDWTNKNVHPSKVVQLGDEVEVMILEIDEGRRRISLGMKQCQANPWEEFAANHNKGDKISG |
| | AVKSITDFGVFVGLPGGIDGLVHLSDLSWTESGEEAVRKYKKGEEVEAVVLAIDVEKERISL |
| | GIKQLEGDPFGNFISVNDKGSLVKGSVKSVDAKGAVIALSDEVEGYLPASEFAADRVEDLTT |
| | KLKEGDEVEAVIVTVDRKNRSIKLSVKAKDAKESREALNSVNAAANANAGTTSLGDLLKAKL |
| | SGEQE-C (SEQ ID NO: 437) |
| N. gonorrhoeae | 5′-ATGGCACTGACCGTAGAACAAAAAGCACAAATCGTTAAAGATTTCCAACGTAAAGAAGG |
| S15 RNA | CGACACCGGCTCTTCTGAAGTACAAGTTGCCCTGTTGACTTTCCGCATCAACGATCTGACCC |
| | CCCACTTCAAAGCCAACCCCAAAGACCACCACAGCCGTCGCGGCCTGTTGAAAATGGTCAGC |
| | CAACGCCGTCGCCTGTTGGTCTATTTGCGCCGTACCCAGCCCGATACGTATCGCGCGTTGAT |
| | TACCCGCTTGGGTCTGCGTAAATAA-3′ (SEQ ID NO: 438) |
| N. gonorrhoeae | N-MALTVEQKAQIVKDFQRKEGDTGSSEVQVALLTFRINDLTPHFKANPKDHHSRRGLLKMV |
| S15 Protein | SQRRRLLVYLRRTQPDTYRALITRLGLRK-C (SEQ ID NO: 439) |
| B. pertussis | 5′-ATGGCCAATACCGCCCAAGCCCGCAAGCGCGCTCGCCAATCGGTCCAGCGCAACAAGCA |
| S20 RNA | CAACTCCAGCCTGCGCTCGATGCTGCGCACCGCCATCAAGCGCGTTCGCCAATCCATCGCCA |
| | CTGGCGATAAGGCTGCTGCTGGCGAAACGCTGCGTAAAGCCACGAGCGTGATTGACAGCGTG |
| | GCTGACAAGAACATCATCCACAAGAACAAGGCTGCTCGCCACAAGAGCCGCCTGGCTGCCGC |
| | CGTCAAGGCGCTGGCCTAA-3′ (SEQ ID NO: 440) |
| B. pertussis | N-MANTAQARKRARQSVQRNKHNSSLRSMLRTAIKRVRQSIATGDKAAAGETLRKATSVIDS |
| S20 Protein | VADKNIIHKNKAARHKSRLAAAVKALA-C (SEQ ID NO: 441) |
| B. pertussis | 5′-ATGCTGGTGATTCGTCTGGCCCGCGGTGGCTCGAAGAAGCGTCCGTTTTACAACCTGGT |
| S16 RNA | AGCTACCGATTCGCGCAATCGTCGCGATGGCCGTTTCGTCGAGCGCGTTGGCTTTTACAACC |
| | CGGTTGCCGCTGAAGGCACCGAGAATCTGCGCATCGCCCTGGACCGCGTGCAATACTGGACC |
| | GGCAACGGCGCGCTGCTGTCGCCCGCCGTCGAGCGCCTGGTCAAGGAATACTCGGCCAAGGT |
| | TTCGGCTGCTGCCTGA-3′ (SEQ ID NO: 442) |
| B. pertussis | N-MLVIRLARGGSKKRPFYNLVATDSRNRRDGRFVERVGFYNPVAAEGTENLRIALDRVQYW |
| S16 Protein | TGNGALLSPAVERLVKEYSAKVSAAA-C (SEQ ID NO: 443) |
| B. pertussis | 5′-ATGTCTTCCGTTTCCACCTCCGCCATCCTTGGCGGCGAAAACTTCGCCGACCTGTTCGC |
| S1 RNA | AGAAAGCCTCAAGAGCCAGGACATGAAGTCCGGCGAGGTCATCAGCGCAGAAGTCGTGCGCG |
| | TCGACCACAACTTCGTGGTCGTCAACGCCGGCCTGAAGTCCGAAGCGCTGATTCCCCTGGAA |
| | GAGTTCCTCAACGACCAGGGCGAACTCGAAGTTCAACCCGGCGACTTCGTCTCGGTGGCGAT |
| | CGATTCGCTGGAGAACGGCTACGGCGACACCATCCTGTCGCGCGACCGCGCCAAGCGTCTGT |
| | CGGCCTGGCTGCAACTGGAGCAGGCCCTCGAGAACGGCGAGCTGGTCACCGGCACGATCACC |
| | GGCAAGGTCAAGGGCGGCCTGACCGTCATGACCAACGGCATCCGCGCGTTCCTGCCCGGTTC |
| | GCTGGTCGACCTGCGTCCGGTCAAGGACACCACGCCGTACGAAGGCAAGACCCTCGAATTCA |
| | AGGTCATCAAGCTGGACCGCAAGCGCAACAACGTCGTGCTGTCGCGCCGCCAGGTGCTGGAA |
| | GCCAGCATGGGCGAAGAGCGCCAGAAGCTGCTCGAGACGCTGCACGAAGGCGCGGTGGTCAA |
| | GGGCGTGGTCAAGAACATCACCGACTACGGCGCGTTCGTCGACCTGGGCGGCATCGATGGCC |
| | TGCTGCACATCACCGACATGGCCTGGCGCCGTGTGCGTCACCCGTCCGAAGTCCTGCAAGTG |
| | GGTCAGGAAGTCGAAGCCAAGGTGCTCAAGTTCGACCAGGAAAAGAGCCGCGTCTCCCTGGG |
| | CGTCAAGCAGCTGGGCGAAGATCCGTGGGTGGGCCTGGCTCGCCGCTACCCGCAGGGCACCC |
| | GCCTGTTCGGCAAGGTCACCAACCTGACCGACTACGGCGCGTTCGTCGAAGTCGAAGCCGGC |
| | ATCGAAGGCCTGGTGCACGTGTCCGAAATGGACTGGACCAACAAGAACGTCGATCCGCGCAA |
| | GGTCGTGACCCTGGGCGAAGAAGTCGAAGTCATGGTCCTGGAAATCGACGAAGACCGTCGCC |
| | GCATTTCGCTGGGCATGAAGCAGTGCCGCCAGAACCCGTGGGAAGAGTTCGCCACCAACTTC |
| | AAGCGTGGTGACAAGGTCCGCGGCGCCATCAAGTCGATCACCGACTTCGGCGTGTTCGTCGG |
| | CCTGCCCGGCGGCATCGACGGCCTGGTCCATCTGTCCGACCTGTCGTGGACGGAATCGGGCG |
| | AGGAAGCCGTGCGCAACTTCAAGAAGGGCGACGAGCTGGAAGCCGTGGTGCTGGGCATCGAT |
| | ACCGAGAAAGAGCGCATCTCGCTGGGTATCAAGCAGCTCGAAGGCGACCCGTTCAACAACTT |
| | CGTTGCCACGCACGACAAGGGCGCCGTTGTTCCGGGCACCATCAAGTCGGTCGAGCCCAAGG |
| | GCGCCGTGATCACCCTGTCGGTGGATGTGGAAGGCTACCTGCGCGCCTCCGAGATCTCCTCG |
| | GGCCGCGTCGAAGACGCTACCACCGTGCTGAAGGCTGGCGAGAACATCGAAGCCATGATCGT |
| | CAACATCGACCGCAAGGCGCGTTCGATCCAGCTGTCGATCAAGGCCCGCGATAACGCCGAGA |
| | CGGCCGAGACCATCCAGCGCATGTCCGAGGCGAGCGCTTCGTCGGGTACGACGAACTTGGGC |
| | GCGCTGCTCAAGGCCAAGCTGGACCAACAGCGCAACGACGGTTGA-3′ |
| | (SEQ ID NO: 444) |
| B. pertussis | N-MSSVSTSAILGGENFADLFAESLKSQDMKSGEVISAEVVRVDHNFVVVNAGLKSEALIPL |
| S1 Protein | EEFLNDQGELEVQPGDFVSVAIDSLENGYGDTILSRDRAKRLSAWLQLEQALENGELVTGTI |
| | TGKVKGGLTVMTNGIRAFLPGSLVDLRPVKDTTPYEGKTLEFKVIKLDRKRNNVVLSRRQVL |
| | EASMGEERQKLLETLHEGAVVKGVVKNITDYGAFVDLGGIDGLLHITDMAWRRVRHPSEVLQ |
| | VGQEVEAKVLKFDQEKSRVSLGVKQLGEDPWVGLARRYPQGTRLFGKVTNLTDYGAFVEVEA |
| | GIEGLVHVSEMDWTNKNVDPRKVVTLGEEVEVMVLEIDEDRRRISLGMKQCRQNPWEEFATN |
| | FKRGDKVRGAIKSITDFGVFVGLPGGIDGLVHLSDLSWTESGEEAVRNFKKGDELEAVVLGI |
| | DTEKERISLGIKQLEGDPFNNFVATHDKGAVVPGTIKSVEPKGAVITLSVDVEGYLRASEIS |
| | SGRVEDATTVLKAGENIEAMIVNIDRKARSIQLSIKARDNAETAETIQRMSEASASSGTTNL |
| | GALLKAKLDQQRNDG-C(SEQ ID NO: 445) |
| B. pertussis | 5′-ATGTCTGTAGCTGACATCAAAAAATCCGAAATCGTCGCGCAATTCCAACGCGCTCAAGG |
| S15 RNA | CGATACCGGCTCCCCTGAAGTTCAGGTGGCACTGCTTACCGCTCGCATCAACGAACTGACCG |
| | GTCACTTCAAAGAACACATGAAGGACCACCACTCGCGCCGCGGCCTGCTGCGCATGGTCAGC |
| | CGTCGTCGCAAGCTGCTCGACTATCTCAAGGGCCGCAATCCCGATTCGTACCGCGCACTGAT |
| | CGAAAAACTCGGTCTGCGCAAGTGA-3′ (SEQ ID NO: 446) |
| B. pertussis | N-MSVADIKKSEIVAQFQRAQGDTGSPEVQVALLTARINELTGHFKEHMKDHHSRRGLLRMV |
| S15 Protein | SRRRKLLDYLKGRNPDSYRALIEKLGLRK-C(SEQ ID NO: 447) |
| A. faecalis | 5′-ATGGCTAATACCGCACAAGCACGCAAGCGCGCCCGCCAAGCAGTGGCTCGCAACAAACA |
| S20 RNA | CAACGCCAGCATCCGTTCCATGCTGCGTACCGCTATCAAGCGCGTTCGCCAGGCTATCGAAG |
| | CCGGTGACAAAACTGCTGCTAACGAAGTTTTCCGCAAAGCGACCAGCATCATTGATCGCGTT |
| | GCCGACAAGAACATCATTCACAAGAACAAGGCTGCCCGCCACAAGAGCCGCCTGTCTGCTGC |
| | TATCAAGGCCCTGGCCTGA-3′ (SEQ ID NO: 448) |
| A. faecalis | N-MANTAQARKRARQAVARNKHNASIRSMLRTAIKRVRQAIEAGDKTAANEVFRKATSIIDR |
| S20 Protein | VADKNIIHKNKAARHKSRLSAAIKALA-C(SEQ ID NO: 449) |
| A. faecalis | 5′-ATGGTGGTAATTCGCTTGGCCCGTGGTGGCTCCAAAAAGCGTCCTTTTTACAACGTAGT |
| S16 RNA | AGCCGCCGAGGCGAGCCAACGCCGTGATGGTCGCTTCATCGAACGTCTGGGTTTCTACAACC |
| | CCGTCGCTGGCGAAGGTCATGAAAACCTGCGTCTGCAAATGGACCGCGTTAAGCACTTCACC |
| | GACAACGGTGCTCAGGTTTCGCCTGCTGTGGCTCGCCTGTTGAAAGAATACTCGGCTAAGGT |
| | TGCTTCGGCTGCCTAA-3′ (SEQ ID NO: 450) |
| A. faecalis | N-MVVIRLARGGSKKRPFYNVVAAEASQRRDGRFIERLGFYNPVAGEGHENLRLQMDRVKHF |
| S16 Protein | TDNGAQVSPAVARLLKEYSAKVASA-C (SEQ ID NO: 451) |
| A. faecalis | 5′-ATGTCCACTAACCTTCACACAGACGCAACGGGCGGCGAAAGCTTTGCCGATTTGTTTGC |
| S1 RNA | AGAAAGCATTAAAAACCAAGACCTGAAAAGCGGCGAAGTTATCTCCGCCGAGGTTCTGCGTA |
| | TTGACCACAACTACGTTCTGGTCAATGCTGGCCTGAAGTCCGAAGCACTGATCCCTCTGGAA |
| | GAGTTCCTGAACGATCAGGGCGAGCTGGAAGTTCAAGCTGGTGACTTTGTTTCCGTGGCAAT |
| | CGACGCCTTTGAAAACGGCTACGGCGACACCATCCTGTCCCGCGACCGCGCCAAGCGTCTGT |
| | CGGCCTGGTTGTCCCTCGAGCAAGCGCTTGAGTCCGGCGAACTGGTCAACGGTACCATCACT |
| | GGTAAGGTCAAGGGCGGTCTGACTGTCATGACCAACGGCATCCGTGCATTCTTGCCCGGTTC |
| | GCTGGTTGATCTGCGTCCTGTCAAGGACACCACGCCATACGAAGGCAAGACCCTGGAATTCA |
| | AGGTTATCAAGCTCGATCGCAAGCGTAACAACGTCGTGTTGTCGCGTCGCGCTGTGCTGGAA |
| | GCCTCCATGGGCGAAGAGCGTCAGAAACTGCTGGAAACCCTGTCCGAAGGCGCTATCGTCAA |
| | AGGTATCGTCAAGAATATCACCGATTACGGTGCGTTCGTGGATCTGGGCGGCATCGACGGTC |
| | TGCTGCACATCACCGACATGGCATGGCGCCGTGTTCGTCACCCATCCGAAGTTCTGCAGGTG |
| | GGTCAGGAAATCGAAGCCAAGGTCCTCAAGTTCGATCAGGAAAAGAGCCGCGTCTCCCTGGG |
| | CGTCAAGCAGCTGGGCGAAGATCCATGGATCGGTCTGGCACGTCGCTACCCACAAAACACCC |
| | GTTTGTTCGGCAAGGTTACCAACCTGACCGACTACGGTGCATTTGTTGAAGTGGAAGACGGT |
| | ATCGAAGGTCTGGTTCACGTATCCGAAATGGATTGGACCAACAAGAACGTTGACCCACGCAA |
| | GGTTGTGACCTTGGGTGAAGAAGTCGAAGTCATGGTTCTGGAAATCGACGAAGATCGTCGTC |
| | GTATCTCCTTGGGCATGAAGCAGTGCCGTGCCAACCCATGGGAAGATTTCGCCATCAACTTC |
| | AAGCGCGGCGACAAGGTTCGCGGTGGCATCAAGTCCATCACCGACTTCGGCGTGTTTGTTGG |
| | TCTGCCAGGCGGCATCGACGGTCTGGTTCACCTGTCCGACCTGTCCTGGTCCGAGACTGGCG |
| | AAGAAGCCGTTCGCAACTTCAAGAAGGGCGACGAAGTTGACGCCGTGGTTCTGGGCATCGAC |
| | ACCGACAAAGAGCGCATTTCCCTGGGTATCAAACAGCTGGAAGGCGATCCGTTCAACAACTT |
| | CGTAGCTACCTACGACAAGGGTGCTGTAGTTAGCGGCGTCGTCAAGTCGGTAGAAGCCAAGG |
| | GTGCTGTTGTTACTCTGTCGCTGGACGTTGAAGGCTACCTGCGCGCCTCCGAGATCTCCTCG |
| | GGTCGTGTTGAAGATGCCACGACGGCTCTGAAAGAAGGTCAGGAAATCGAAACCATGATCTT |
| | GAGCGTGGATCGCAAGACCCGTTCGATTCAGTTGTCCATCAAGGCTCGTGATACCGCCGAAA |
| | CCGCTCAATCCATGGCCCGCCTGACCGAAGCCAGCGCTTCGTCCGGTACGACCAATCTGGGT |
| | GCTCTGCTGCGTGCCAAGCTGGACCAGCAGCAACGTGACGACGGTTAA-3′ |
| | (SEQ ID NO: 452) |
| A. faecalis | N-MSTNLHTDATGGESFADLFAESIKNQDLKSGEVISAEVLRIDHNYVLVNAGLKSEALIPL |
| S1 Protein | EEFLNDQGELEVQAGDFVSVAIDAFENGYGDTILSRDRAKRLSAWLSLEQALESGELVNGTI |
| | TGKVKGGLTVMTNGIRAFLPGSLVDLRPVKDTTPYEGKTLEFKVIKLDRKRNNVVLSRRAVL |
| | EASMGEERQKLLETLSEGAIVKGIVKNITDYGAFVDLGGIDGLLHITDMAWRRVRHPSEVLQ |
| | VGQEIEAKVLKFDQEKSRVSLGVKQLGEDPWIGLARRYPQNTRLFGKVTNLTDYGAFVEVED |
| | GIEGLVHVSEMDWTNKNVDPRKVVTLGEEVEVMVLEIDEDRRRISLGMKQCRANPWEDFAIN |
| | FKRGDKVRGGIKSITDFGVFVGLPGGIDGLVHLSDLSWSETGEEAVRNFKKGDEVDAVVLGI |
| | DTDKERISLGIKQLEGDPFNNFVATYDKGAVVSGVVKSVEAKGAVVTLSLDVEGYLRASEIS |
| | SGRVEDATTALKEGQEIETMILSVDRKTRSIQLSIKARDTAETAQSMARLTEASASSGTTNL |
| | GALLRAKLDQQQRDDG-C (SEQ ID NO: 453) |
| A. faecalis | 5′-ATGGCAGTTGCAGATATCAAAAAAGTCGATATCGTTTCCGAGTTTCAGCGTAAACAAGG |
| S15 RNA | CGATACAGGCTCCCCAGAAGTGCAAGTGGCTTTGCTGACCGCTCGCATCAACGAATTGACCG |
| | GCCACTTCAAGGCTCACAACAAGGATCACCACTCCCGTCGTGGTCTGTTGCGCATGGTTAGC |
| | CGTCGTCGCAAATTGCTGGATTACCTGAAAGGTCGTAATCCAGACGCTTACCGTGCTTTGAT |
| | CGAAAAACTCGGTCTGCGTAAGTGA-3′ (SEQ ID NO: 454) |
| A. faecalis | N-MAVADIKKVDIVSEFQRKQGDTGSPEVQVALLTARINELTGHFKAHNKDHHSRRGLLRMV |
| S15 Protein | SRRRKLLDYLKGRNPDAYRALIEKLGLRK-C(SEQ ID NO: 455) |
| V. cholerae | 5′-TTGGCAAATAACAAATCTGCTAAGAAGCGCGCTATCCAAGCTGAGAAACGTCGCCAGCA |
| S20 RNA | CAATGCTAGCCGTCGTTCTATGATGCGCACTTACATGAAGAAAACTGTTGCTGCAATCGCTG |
| | CTGGCGACAAAGAAGCTGCAACTGCTGCATTCGCTGTAGTTACACCAATCCTAGACCGCATG |
| | GCGACTAAAGGCCTTATTCACAAGAATAAAGCAGCTCGTCACAAGTCTCGCTTCTTTGCTGC |
| | TATCAACGCTCTGTAA-3′ (SEQ ID NO: 456) |
| V. cholerae | N-LANNKSAKKRAIQAEKRRQHNASRRSMMRTYMKKTVAAIAAGDKEAATAAFAVVTPILDR |
| S20 Protein | MATKGLIHKNKAARHKSRFFAAINAL-C (SEQ ID NO: 457) |
| V. cholerae | 5′-ATGGTAACCATTCGTTTGGCACGTCACGGCGCGAAAAAGCGTCCATTCTATCAAATCGT |
| S16 RNA | TGTAGCTGACAGCCGCAACTCAGCAACTGGCCGTTTCATCGAGAAAGTAGGTTTCTTTAACC |
| | CTACTGCTACTGGTCAAGAAGAAGGCCTGCGTCTAGACCTAGATCGCGTGAACCACTGGGTT |
| | TCACAAGGCGCATCTCTGTCTGATCGCGTAGCACAGCTAGTTAAAACCGCTCAAAAAGCGGC |
| | TTAA-3′ (SEQ ID NO: 458) |
| V. cholerae | N-MVTIRLARHGAKKRPFYQIVVADSRNSATGRFIEKVGFFNPTATGQEEGLRLDLDRVNHW |
| S16 Protein | VSQGASLSDRVAQLVKTAQKAA-C (SEQ ID NO: 459) |
| V. cholerae | 5′-ATGACTGAATCTTTTGCTCAACTCTTTGAAGAGTTTCTGAACGAGACCCAATTCCAACA |
| S1 RNA | AGGTACTATCGTTAAAGGTACTGTAGTAGCAATCGAGAACGGCTACGTTCTGGTTGATGCTG |
| | GCCTGAAGTCTGAGTCTGCTATCCCTGCTGAACAATTCAAGAACGCTGCTGGCGAACTAGAA |
| | GTTCAAGTCGGTTCTCAAGTAGACGTAGCTCTGGACGCGGTTGAAGATGGTTTCGGCGAAAC |
| | TCAACTTTCTCGTGAGAAAGCGAAGCGCCATGAAGCTTGGATCGTACTTGAAAAAGCTTACG |
| | AAGAAGCTGCAACTGTTATCGGTATCATTAACGGTAAAGTTAAAGGTGGTTTCACTGTTGAA |
| | CTGAACGGTATCCGTGCGTTCCTGCCAGGTTCTCTGGTTGACGTACGTCCAATCCGTGACAC |
| | TGCTCACTTAGAAAACAAAGAGCTAGAGTTCAAAGTTATCAAGCTAGACCAGAAGCGTAACA |
| | ACGTTGTTGTTTCTCGTCGTGCAGTTATCGAATCTGAAAGCAGCGTAGAGCGTGATGAACTG |
| | CTTGAAACTCTGCAAGAAGGTACTGAAGTTAAAGGTATCGTTAAGAACCTGACTGACTACGG |
| | TGCGTTCGTTGATCTGGGCGGCGTTGACGGTCTGCTACACATCACTGACATGGCTTGGAAGC |
| | GCGTTAAGCATCCATCAGAAATCGTTAACGTTGGTGACGAGATCCTAGTTAAAGTACTGAAG |
| | TTCGATCGTGATCGTACTCGCGTATCTCTAGGTCTGAAACAGCTAGGCGAAGATCCATGGGT |
| | AGCAATCGCTAAGCGTTACCCAGAAGGTCACAAACTGTCTGGCCGTGTAACTAACCTGACTG |
| | ACTACGGCTGCTTCGTTGAAATCGAAGAAGGCGTTGAAGGTCTAGTACACGTTTCTGAAATG |
| | GACTGGACGAACAAGAACATCCACCCATCTAAAGTTGTTAATGTTGGCGACGAAGTTGAGGT |
| | TATGGTTCTGGATATCGACGAAGAGCGTCGTCGTATCTCTCTAGGCCTGAAACAGTGTAAAG |
| | CTAACCCATGGCAATCATTCGCTGAAGCGCAAGCTAAAGGCGACAAAGTAACTGGTAAGATC |
| | AAGTCTATCACTGACTTTGGTATCTTCATCGGTCTAGAAGGCGGTATCGACGGTCTGGTTCA |
| | CCTGTCTGACATCTCTTGGAACGTACCAGGAGAAGAAGCTGTTCGTGAATTCAAGAAAGGCG |
| | ACGAAATCTCTGCAGTTGTTCTAGCAGTAGACGCTGAGCGTGAGCGTATTTCTCTGGGCATC |
| | AAGCAAATGGAAAACGATCCATTCAACGCTTATGTTTCAGACAACAAGAAAGGTGCTCTGGT |
| | TAACGGTACTGTAACTGCAGTTGACGCGAAAGGCGCAACCATCGAGCTAGAAGACGGCGTTG |
| | AAGGTTACATCCGCGCTTCTGAAGTATCACGTGACCGTATCGAAGATGCATCACTGATCCTG |
| | AACGTTGGTGACAAAGTTGAAGCGAAATTTACTGGTGTTGACCGTAAAAACCGCGTAATCAA |
| | CCTGTCTATCAAAGCGAAAGATGAAGCTGAAGAGCAAGAAGTAATGGCAACTCTGAACAAGC |
| | AAGATGAAGCTGCGTTCGGTAATGCTATGGCTGACGCTTTCAAAGCAGCGAAAGGCGAATA |
| | A-3′ (SEQ ID NO: 460) |
| V. cholerae | N-MTESFAQLFEEFLNETQFQQGTIVKGTVVAIENGYVLVDAGLKSESAIPAEQFKNAAGEL |
| S1 Protein | EVQVGSQVDVALDAVEDGFGETQLSREKAKRHEAWIVLEKAYEEAATVIGIINGKVKGGFTV |
| | ELNGIRAFLPGSLVDVRPIRDTAHLENKELEFKVIKLDQKRNNVVVSRRAVIESESSVERDE |
| | LLETLQEGTEVKGIVKNLTDYGAFVDLGGVDGLLHITDMAWKRVKHPSEIVNVGDEILVKVL |
| | KFDRDRTRVSLGLKQLGEDPWVAIAKRYPEGHKLSGRVTNLTDYGCFVEIEEGVEGLVHVSE |
| | MDWTNKNIHPSKVVNVGDEVEVMVLDIDEERRRISLGLKQCKANPWQSFAEAQAKGDKVTGK |
| | IKSITDFGIFTGLEGGIDGLVHLSDISWNVPGEEAVREFKKGDEISAVVLAVDAERERISLG |
| | IKQMENDPFNAYVSDNKKGALVNGTVTAVDAKGATIELEDGVEGYIRASEVSRDRIEDASLI |
| | LNVGDKVEAKFTGVDRKNRVINLSIKAKDEAEEQEVMATLNKQDEAAFGNAMADAFKAAKG |
| | E-C (SEQ ID NO: 461) |
| V. cholerae | 5′-ATGTCTCTGAATGCAGAAACTAAAGCAGCAATCGTTGCTGAATATGCACGTTGCGAAAA |
| S15 RNA | TGACACAGGTTCACCAGAAGTACAGATCGCTCTACTGACTGCTTCTATCAACCACCTGCAAG |
| | GTCACTTCCAAGCACACAAAGGCGATCACCACAGCCGTCGTGGTCTGCTACGTATGGTTTCA |
| | AGCCGTCGTAAACTGCTGGACTACCTGAAAGGTAAAGATCTGTCTCGTTACCAAGATCTGAT |
| | CAAACGTCTAGGCCTGCGTCGTTAA-3′ (SEQ ID NO: 462) |
| V. cholerae | N-MSLNAETKAAIVAEYARCENDTGSPEVQIALLTASINHLQGHFQAHKGDHHSRRGLLRMV |
| S15 Protein | SSRRKLLDYLKGKDLSRYQDLIKRLGLRR-C (SEQ ID NO: 463) |
| A. baumannii | 5′-GTGGCAAACTCTGCTCAAGCTAAAAAACGTGCGCGCCAAAACGTTAAAGCGCGTAAACA |
| S20 RNA | CAACGCAAGCTTGCGTTCTATGGTTCGTACTTACATCAAACGTACTTTAAGTGCAATCGCTG |
| | GTGGTGATTATGCTGTTGCTACAGAAGCTTACAAAAAAGCTGTTCCTGTAATCGACCGCATG |
| | GCTGATAAAGGCATCATCCACAAAAATAAAGCTGCTCGTCATAAGAGCCGTTTAAATGCTCA |
| | AGTTAAAGCTTTAGCTAACTAA-3′ (SEQ ID NO: 464) |
| A. baumannii | N-VANSAQAKKRARQNVKARKHNASLRSMVRTYIKRTLSAIAGGDYAVATEAYKKAVPVIDR |
| S20 Protein | MADKGIIHKNKAARHKSRLNAQVKALAN-C (SEQ ID NO: 465) |
| A. baumannii | 5′-ATGGTTGTTATTCGTTTAGCACGTGGCGGCGCAAAAAAACGTCCATTCTATCAAATCGT |
| S16 RNA | TGTGACTGATAGCCGCAATGCACGTGATGGTCGTTTCATCGAGCGTATCGGTTTCTTTAACC |
| | CAACTGCACAAGGTCAAGCAGAAAAACTTCGTTTAGACGCAGACCGTTTTGCTCACTGGGTT |
| | TCTCAAGGTGCTCAACCTTCTGAACGTGTTGCTTCTTTGGCTGCTCAAGCTAAAAAAGCTAC |
| | TGCATAA-3′ (SEQ ID NO: 466) |
| A. baumannii | N-MVVIRLARGGAKKRPFYQIVVTDSRNARDGRFIERIGFFNPTAQGQAEKLRLDADRFAHW |
| S16 Protein | VSQGAQPSERVASLAAQAKKATA-C (SEQ ID NO: 467) |
| A. baumannii | 5′-ATGACCGAATCTTTTGCAGCCCTCTTTGAAGAAAGTGAATTAAACCTCAACGTTGAAAA |
| S1 RNA | GGGTGCAGTCATCCAAGGTGTTGTTGTAAACATCGATAGCGACTGGGTTACTGTTGACACTG |
| | GCCTTAAATCAGAAGGCATTGTTGACCGTGCTGAATTTTTAAATGAACAACGTGAACTTGAA |
| | GTTCAGGTTGGTGACACTGTTGACGTAGTTGTTGAAGCTCTTGACAACGGTATGGGTCAAAC |
| | AGTTTTATCACGTGAAAAAGCTAAACGTGCTGAAACTTGGACTAAACTTGAAAAAATCTTTG |
| | AAGATGGCGAAATCGTTACTGGTGTTATCTCTGGTAAAGTTAAAGGCGGTTTCACTGTTGAC |
| | ATCGGTCCTGTTCGTGCGTTCTTACCAGGTTCATTAGTTGACACTCGTCCTATCCGTGACAC |
| | TACTCACCTTGAAGGTAAAGAGTTAGAGTTTAAAGTAATCAAACTTGATGCTAAACGTAACA |
| | ACGTTGTTGTATCTCGTCGTGCTGTTATGGAAGCTGAATCTTCTGCTGACCGTGAAGCATTA |
| | CTTGCTCAACTTGAAGAAGGTCAAACAGTTACAGGTACTATCAAGAACCTTACTGATTACGG |
| | TGCATTCGTTGATCTTGGCGGTATTGATGGTCTTCTTCATATCACAGATATGGCTTGGAAGC |
| | GTATCAAGCACCCTTCAGAAGTTGTTGAAGTTGGTCAAGAAGTTACTGTTAAAGTACTTAAA |
| | TTTGACCGTGAACGTAACCGCGTATCTTTAGGCCTTAAACAATTAGGCGAAGATCCATGGTT |
| | AGCGATCATGAGCCGTTACCCTAAAGGTTCTATCGTTAAAGCACGTGTTACTAACTTAACTG |
| | ACTACGGTTGTTTCGCTGAAATCGCTGAAGGCGTTGAAGGTTTAGTACACGTTTCTGAAATG |
| | GACCACACTAACAAAAACATCCACCCATCTAAAGTTGTTCAGATCGGTGACGAAGTTGATGT |
| | TATGGTTCTTGAAGTTGACGAAGAACGTCGTCGTATCAGCCTTGGTATCAAACAAACTCGTG |
| | CTAACCCATGGGAAGAGTTTGCTAAGTCTCATGAGAAAGGCGAAAAAGTTTCAGGCACAATC |
| | AAGTCTATCACTGACTTTGGTATCTTCATTGGTTTAAATGGCGGTATCGACGGTCTAGTTCA |
| | CTTGTCTGATATTTCTTGGAACGAACAAGGCGAAGAAGCTATCCGTCGTTACAAGAAAGGTG |
| | ACACTGTTGAAGCAGTTATCTTGTCTGTAGACGCTGAAGGTAACCGTATCAGCCTTGGTATC |
| | AAGCAATTGAACAGCGATCCGTTCAATGATTTCTTAGCTGCTAACGAACGTGGTGCTTTAGT |
| | TAAAGGTACTGTGACTGCAGTTGATGCTCGTGGCGCAACTGTTAAGTTAGCTGACGAAGTAG |
| | AAGCTACTCTTAAAGCTTCTGAAATCAACCGTGACCGCGTTGAAGATGCAACTAAATTCTTA |
| | GAAGTTGGTCAAGAAGTTGAAGCTAAAATCATCAACGTTGATCGTAAATCTCGCTCTATCAA |
| | CTTGTCTATCAAAGCGAAAGACGAAGCTGAAGAGAAAGAAGCAGTAGCTAACTTACGTACAG |
| | CATCAGCTTCTCAAGAAAATGGTCCTAAGACTATTGGTGACTTGATCAAAGCGCAAATGAAG |
| | TAA-3′ (SEQ ID NO: 468) |
| A. bauniannii | N-MTESFAALFEESELNLNVEKGAVIQGVVVNIDSDWVTVDTGLKSEGIVDRAEFLNEQREL |
| S1 Protein | EVQVGDTVDVVVEALDNGMGQTVLSREKAKRAETWTKLEKIFEDGEIVTGVISGKVKGGFTV |
| | DIGPVRAFLPGSLVDTRPIRDTTHLEGKELEFKVIKLDAKRNNVVVSRRAVMEAESSADREA |
| | LLAQLEEGQTVTGTIKNLTDYGAFVDLGGIDGLLHITDMAWKRIKHPSEVVEVGQEVTVKVL |
| | KFDRERNRVSLGLKQLGEDPWLAIMSRYPKGSIVKARVTNLTDYGCFAEIAEGVEGLVHVSE |
| | MDHTNKNIHPSKVVQIGDEVDVMVLEVDEERRRISLGIKQTRANPWEEFAKSHEKGEKVSGT |
| | IKSITDFGIFTGLNGGIDGLVHLSDISWNEQGEEAIRRYKKGDTVEAVILSVDAEGNRISLG |
| | IKQLNSDPFNDFLAANERGALVKGTVTAVDARGATVKLADEVEATLKASEINRDRVEDATKF |
| | LEVGQEVEAKIINVDRKSRSINLSIKAKDEAEEKEAVANLRTASASQENGPKTIGDLIKAQM |
| | K-C (SEQ ID NO: 469) |
| A. bauniannii | 5′-ATGGCTTTAACTAATGCAGACCGCGCAGAGATCATTGCTAAATTTGCTCGCGCTGAAAA |
| S15 RNA | CGACACTGGTTCACCAGAAGTACAAGTAGCTTTATTGACTGCTCAAATCAATGATTTACAAG |
| | GTCACTTTAAAGCTCACAAACACGACCACCATAGCCGTCGCGGTTTGATCCGTATGGTTAAC |
| | CAACGTCGTAAATTACTTGACTACTTAAATGGTAAAGACCACGAGCGTTACACTGCTTTGAT |
| | CGGTGCTTTAGGTCTACGTCGTTAA-3′ (SEQ ID NO: 470) |
| A. bauniannii | N-MALTNADRAEIIAKFARAENDTGSPEVQVALLTAQINDLQGHFKAHKHDHHSRRGLIRMV |
| S15 Protein | NQRRKLLDYLNGKDHERYTALIGALGLRR-C(SEQ ID NO: 471) |
| P. aeruginosa | 5′-GTGGCCAACACACCTTCCGCCAAAAAACGCGCCAAACAGGCTGAGAAGCGTCGCAGCCA |
| S20 RNA | CAACGCCAGCCTGCGCTCCATGGTGCGCACCTACATCAAGAACGTCGTGAAAGCCATCGACG |
| | CCAAGGACCTGGAAAAAGCCCAGGCCGCCTTCACCGCCGCTGTACCGGTGATCGACCGCATG |
| | GCTGACAAAGGCATCATCCACAAGAACAAGGCTGCTCGTCATAAGAGCCGTCTGAGCGGCCA |
| | CATCAAGGCCCTCAGCACCGCTGCCGCCTAA-3′ (SEQ ID NO: 472) |
| P. aeruginosa | N-VANTPSAKKRAKQAEKRRSHNASLRSMVRTYIKNVVKAIDAKDLEKAQAAFTAAVPVIDR |
| S20 Protein | MADKGIIHKNKAARHKSRLSGHIKALSTAAA-C (SEQ ID NO: 473) |
| P. aeruginosa | 5′-ATGGTAACCATCCGTCTTGCTCGTGGCGGCTCCAAAAAGCGCCCCTTCTACCACCTGAC |
| S16 RNA | CGTGACCAACAGCCGCAATGCGCGCGATGGTCGCTTCGTCGAGCGCATCGGTTTCTTCAACC |
| | CGGTTGCGACTGGTGGCGAAGTGCGTCTGTCCGTCGACCAGGAGCGTGCTACCTACTGGCTC |
| | GGCCAGGGTGCACAGCCGTCTGAGCGCGTTGCTCAGCTGCTCAAGGACGCTGCCAAGGCAAA |
| | CGCCTAA-3′ (SEQ ID NO: 474) |
| P. aeruginosa | N-MVTIRLARGGSKKRPFYHLTVTNSRNARDGRFVERIGFFNPVATGGEVRLSVDQERATYW |
| S16 Protein | LGQGAQPSERVAQLLKDAAKANA-C (SEQ ID NO: 475) |
| P. aeruginosa | 5′-ATGAGCGAAAGCTTCGCAGAACTCTTTGAAGAAAGTCTGAAATCCCTCGACATGCAGCC |
| S1 RNA | GGGTGCCATCATCACCGGCATCGTGGTCGACATCGATGGTGACTGGGTCACCGTCCATGCCG |
| | GTCTGAAATCCGAGGGCGTCATCCCGGTCGAGCAGTTCTACAACGAACAGGGCGAGCTGACC |
| | ATCAAGGTGGGTGACGAAGTCCACGTCGCACTGGACGCGGTAGAAGACGGCTTTGGCGAGAC |
| | CAAGCTGTCCCGCGAGAAAGCCAAGCGCGCCGAGAGCTGGATTGTTCTGGAAGCGGCTTTCG |
| | CTGCCGACGAAGTGGTCAAGGGCGTCATCAACGGCAAGGTCAAGGGCGGTTTCACCGTCGAC |
| | GTCAACGGCATCCGCGCGTTCCTGCCGGGTTCTCTGGTCGACGTTCGTCCGGTTCGCGACAC |
| | CACCCACCTGGAAGGCAAAGAGCTCGAGTTCAAGGTCATCAAGCTCGACCAGAAGCGCAACA |
| | ACGTTGTCGTTTCCCGCCGCAGCGTCCTGGAAGCCGAGAACAGCGCCGAGCGTGAAGCTCTG |
| | CTGGAATCGCTGCAGGAAGGCCAGCAGGTCAAAGGTATCGTCAAGAACCTCACCGACTACGG |
| | CGCATTCGTGGACCTGGGCGGCGTAGACGGCCTGCTACACATCACCGACATGGCCTGGAAGC |
| | GCATCAAGCATCCGTCCGAGATCGTCAACGTTGGCGACGAGATCGACGTCAAGGTCCTGAAG |
| | TTCGACCGCGAGCGCAACCGTGTATCCCTGGGCCTGAAGCAACTGGGCGAAGACCCGTGGGT |
| | TGCCATCAAGGCGCGTTACCCGGAAGGTACCCGCGTCATGGCCCGCGTCACCAACCTCACCG |
| | ACTACGGCTGCTTCGCCGAGCTGGAAGAGGGCGTGGAAGGCCTGGTACACGTCTCCGAAATG |
| | GACTGGACCAACAAGAACATCCATCCGTCGAAAGTCGTCCAGGTTGGCGATGAAGTGGAAGT |
| | TCAGGTTCTGGACATCGACGAAGAGCGTCGTCGTATCTCCCTGGGTATCAAGCAGTGCAAAT |
| | CCAACCCGTGGGAAGACTTCTCCAGCCAGTTCAACAAGGGTGACCGTATCTCCGGTACCATC |
| | AAGTCGATCACCGACTTCGGTATCTTCATCGGTCTGGACGGCGGCATCGACGGCCTGGTCCA |
| | CCTGTCCGACATCTCCTGGAACGAAGTCGGCGAAGAAGCCGTACGTCGCTTCAAGAAGGGCG |
| | ACGAGCTGGAAACCGTCATCCTGTCGGTCGATCCGGAGCGCGAGCGCATCTCCCTGGGCATC |
| | AAGCAGCTGGAAGACGATCCGTTCTCCAACTACGCGTCCCTGCACGAGAAAGGCAGCATCGT |
| | CCGCGGTACCGTGAAGGAAGTCGACGCCAAGGGCGCTGTCATCAGCCTGGGCGACGACATCG |
| | AAGGTATCCTGAAGGCTTCCGAAATCAGCCGTGACCGCGTCGAAGACGCGCGCAACGTCCTG |
| | AAGGAAGGCGAGGAAGTCGAAGCCAAGATCATCAGCATCGACCGCAAGAGCCGCGTCATCAG |
| | CCTCTCCGTCAAGTCCAAGGACGTCGACGACGAGAAGGACGCAATGAAAGAACTGCGTAAGC |
| | AGGAAGTAGAAAGCGCTGGTCCGACCACCATCGGTGATCTGATCCGTGCTCAGATGGAGAAT |
| | CAGGGCTAA-3′ (SEQ ID NO: 476) |
| P. aeruginosa | N-MSESFAELFEESLKSLDMQPGAIITGIVVDIDGDWVTVHAGLKSEGVIPVEQFYNEQGEL |
| S1 Protein | TIKVGDEVHVALDAVEDGFGETKLSREKAKRAESWIVLEAAFAADEVVKGVINGKVKGGFTV |
| | DVNGIRAFLPGSLVDVRPVRDTTHLEGKELEFKVIKLDQKRNNVVVSRRSVLEAENSAEREA |
| | LLESLQEGQQVKGIVKNLTDYGAFVDLGGVDGLLHITDMAWKRIKHPSEIVNVGDEIDVKVL |
| | KFDRERNRVSLGLKQLGEDPWVAIKARYPEGTRVMARVTNLTDYGCFAELEEGVEGLVHVSE |
| | MDWTNKNIHPSKVVQVGDEVEVQVLDIDEERRRISLGIKQCKSNPWEDFSSQFNKGDRISGT |
| | IKSITDFGIFIGLDGGIDGLVHLSDISWNEVGEEAVRRFKKGDELETVILSVDPERERISLG |
| | IKQLEDDPFSNYASLHEKGSIVRGTVKEVDAKGAVISLGDDIEGILKASEISRDRVEDARNV |
| | LKEGEEVEAKIISIDRKSRVISLSVKSKDVDDEKDAMKELRKQEVESAGPTTIGDLIRAQME |
| | NQG-C (SEQ ID NO: 477) |
| P. aeruginosa | 5′-ATGGCACTGAGCGTTGAAGAAAAAGCGCAGATCGTTAACGAATACAAGCAAGCTGAAGG |
| S15 RNA | CGACACCGGTTCCCCGGAAGTGCAGGTAGCCCTGCTGTCCGCCAACATCAACAAGCTGCAGG |
| | ATCACTTCAAGGCCAACGGCAAGGATCACCATTCCCGCCGTGGTCTGATCCGTATGGTTAAC |
| | CAGCGCCGTAAGCTGCTGGACTACCTGAAGGGCAAAGACGTGTCTCGCTACACTGCCCTGAT |
| | CGGCCGTCTGGGTCTGCGTCGCTAA-3′ (SEQ ID NO: 478) |
| P. aeruginosa | N-MALSVEEKAQIVNEYKQAEGDTGSPEVQVALLSANINKLQDHFKANGKDHHSRRGLIRMV |
| S15 Protein | NQRRKLLDYLKGKDVSRYTALIGRLGLRR-C (SEQ ID NO: 479) |

It is contemplated that variant or mutant forms of the sequences presented herein can also be employed in making and using nucleotide and/or protein constructs of the disclosure. Accordingly, the exemplary sequences presented herein can be modified to contain one, two, three, four, five or more variant residues, as compared to those disclosed herein, and still remain within the scope of the contemplated disclosure. Similarly, it is contemplated that a sequence at least 80% identical, at least 90% identical, at least 95% identical, at least 97% identical, at least 98% identical or at least 99% identical to one or more of the specific sequences recited herein can be employed in the compositions and methods of the instant disclosure.

### REFERENCES

1. Chubukov, V., Gerosa, L., Kochanowski, K. & Sauer, U. Coordination of microbial metabolism. Nat Rev Microbiol 12, 327-40 (2014).
2. de Jong, H., Geiselmann, J. & Ropers, D. Resource Reallocation in Bacteria by Reengineering the Gene Expression Machinery. Trends Microbiol 25, 480-493 (2017).
3. Kaczanowska, M. & Ryden-Aulin, M. Ribosome biogenesis and the translation process in Escherichia coli. Microbiol Mol Biol Rev 71, 477-94 (2007).
4. Rackham, O. & Chin, J. W. A network of orthogonal ribosome x mRNA pairs. Nat Chem Biol 1, 159-66 (2005).
5. Wang, K., Neumann, H., Peak-Chew, S. Y. & Chin, J. W. Evolved orthogonal ribosomes enhance the efficiency of synthetic genetic code expansion. Nature Biotechnology 25, 770 (2007).
6. Neumann, H., Wang, K., Davis, L., Garcia-Alai, M. & Chin, J. W. Encoding multiple unnatural amino acids via evolution of a quadruplet-decoding ribosome. Nature 464, 441 (2010).
7. Schmied, W. H. et al. Controlling orthogonal ribosome subunit interactions enables evolution of new function. Nature 564, 444-448 (2018).
8. Orelle, C. et al. Protein synthesis by ribosomes with tethered subunits. Nature 524, 119-24 (2015).
9. Fried, S. D., Schmied, W. H., Uttamapinant, C. & Chin, J. W. Ribosome Subunit Stapling for Orthogonal Translation in E. coli. Angew Chem Int Ed Engl 54, 12791-4 (2015).
10. Filipovska, A. & Rackham, O. Specialization from synthesis: how ribosome diversity can customize protein function. FEBS Lett 587, 1189-97 (2013).
11. Kurylo, C. M. et al. Endogenous rRNA Sequence Variation Can Regulate Stress Response Gene Expression and Phenotype. Cell Rep 25, 236-248.e6 (2018).
12. Kim, H.-L. et al. Heterogeneous rRNAs are differentially expressed during the morphological development of Streptomyces coelicolor. FEMS Microbiology Letters 275, 146-152 (2019).
13. Deusser, E. & Wittmann, H.-G. Biological Sciences: Ribosomal Proteins: Variation of the Protein Composition in Escherichia coli Ribosomes as Function of Growth Rate. Nature 238, 269-270 (2019).
14. Moll, I., Resch, A. & Blasi, U. Discrimination of 5′-terminal start codons by translation initiation factor 3 is mediated by ribosomal protein S1. FEBS Lett 436, 213-7 (1998).
15. Kitahara, K. & Miyazaki, K. Revisiting bacterial phylogeny: Natural and experimental evidence for horizontal gene transfer of 16S rRNA. Mob Genet Elements 3, e24210 (2013).
16. Kitahara, K., Yasutake, Y. & Miyazaki, K. Mutational robustness of 16S ribosomal RNA, shown by experimental horizontal gene transfer in Escherichia coli. Proc Natl Acad Sci USA 109, 19220-5 (2012).
17. Asai, T., Zaporojets, D., Squires, C. & Squires, C. L. An Escherichia coli strain with all chromosomal rRNA operons inactivated: complete exchange of rRNA genes between bacteria. Proc Natl Acad Sci USA 96, 1971-6 (1999).
18. Gyorfy, Z. et al. Engineered ribosomal RNA operon copy-number variants of E. coli reveal the evolutionary trade-offs shaping rRNA operon number. Nucleic Acids Research 43, 1783-1794 (2019).
19. Shi, X., Chiu, K., Ghosh, S. & Joseph, S. Bases in 16S rRNA Important for Subunit Association, tRNA binding, and Translocation. Biochemistry 48, 6772-82 (2009).
20. Gutgsell, N. S. & Jain, C. Role of precursor sequences in the ordered maturation of E. coli 23S ribosomal RNA. (2012).
21. Kitahara, K. & Suzuki, T. The ordered transcription of RNA domains is not essential for ribosome biogenesis in Escherichia coli. Mol Cell 34, 760-6 (2009).
22. Sahu, B., Khade, P. K. & Joseph, S. Functional Replacement of Two Highly Conserved Tetraloops in the Bacterial Ribosome. (2012).
23. Mylvaganam, S. & Dennis, P. P. Sequence heterogeneity between the two genes encoding 16S rRNA from the halophilic archaebacterium Haloarcula marismortui. Genetics 130, 399-410 (1992).
24. Ueda, K., Seki, T., Kudo, T., Yoshida, T. & Kataoka, M. Two Distinct Mechanisms Cause Heterogeneity of 16S rRNA. (1999).
25. Yap, W. H., Zhang, Z. & Wang, Y. Distinct Types of rRNA Operons Exist in the Genome of the Actinomycete Thermomonospora chromogena and Evidence for Horizontal Transfer of an Entire rRNA Operon. (1999).
26. Tian, R. M., Cai, L., Zhang, W. P., Cao, H. L. & Qian, P. Y. Rare Events of Intragenus and Intraspecies Horizontal Transfer of the 16S rRNA Gene. in Genome Biol Evol, Vol. 7 2310-20 (2015).
27. Acinas, S. G., Marcelino, L. A., Klepac-Ceraj, V. & Polz, M. F. Divergence and Redundancy of 16S rRNA Sequences in Genomes with Multiple rm Operons. (2004).
28. Prossliner, T., Skovbo Winther, K., Sorensen, M. A. & Gerdes, K. Ribosome Hibernation. Annu Rev Genet 52, 321-348 (2018).
29. Byrgazov, K., Vesper, O. & Moll, I. Ribosome heterogeneity: another level of complexity in bacterial translation regulation. Curr Opin Microbiol 16, 133-9 (2013).
30. Asai, T. et al. Construction and Initial Characterization of Escherichia coli Strains with Few or No Intact Chromosomal rRNA Operons. in J Bacteriol, Vol. 181 3803-9 (1999).
31. Blattner, F. R. et al. The complete genome sequence of Escherichia coli K-12. Science 277, 1453-62 (1997).
32. Mizoguchi, H., Tanaka-Masuda, K. & Mon, H. A simple method for multiple modification of the Escherichia coli K-12 chromosome. Biosci Biotechnol Biochem 71, 2905-11(2007).
33. Li, X. T., Thomason, L. C., Sawitzke, J. A., Costantino, N. & Court, D. L. Positive and negative selection using the tetA-sacB cassette: recombineering and P1 transduction in Escherichia coli. Nucleic Acids Res 41, e204 (2013).
34. Pedelacq, J. D., Cabantous, S., Tran, T., Terwilliger, T. C. & Waldo, G. S. Engineering and characterization of a superfolder green fluorescent protein. Nat Biotechnol 24, 79-88 (2006).
35. Valdar, W. S. Scoring residue conservation. Proteins 48, 227-41 (2002).
36. Deutscher, M. P. & Biology, B. M. Chapter 9 Maturation and Degradation of Ribosomal RNA in Bacteria. Progress in Molecular Biology and Translational Science 85, 369-391 (2019).
37. Shajani, Z., Sykes, M. T. & Williamson, J. R. Assembly of bacterial ribosomes. Annu Rev Biochem 80, 501-26 (2011).
38. Traxler, M. F. et al. The global, ppGpp-mediated stringent response to amino acid starvation in Escherichia coli. Mol Microbiol 68, 1128-48 (2008).
39. Dunstan, M. S., Guhathakurta, D., Draper, D. E. & Conn, G. L. Coevolution of protein and RNA structures within a highly conserved ribosomal domain. Chem Biol 12, 201-6 (2005).
40. Brandman, R., Brandman, Y. & Pande, V. S. Sequence coevolution between RNA and protein characterized by mutual information between residue triplets. PLoS One 7, e30022 (2012).
41. Peterson, J. & Phillips, G. J. New pSC101-derivative cloning vectors with elevated copy numbers. Plasmid 59, 193-201 (2008).
42. Darlington, A. P. S., Kim, J., Jimenez, J. I. & Bates, D. G. Dynamic allocation of orthogonal ribosomes facilitates uncoupling of co-expressed genes. Nature Communications 9, 1-12 (2018).
43. Madeira, F. et al. The EMBL-EBI search and sequence analysis tools APIs in 2019. Nucleic Acids Res 47, W636-w641 (2019).
44. Noeske, J. et al. High-resolution structure of the Escherichia coli ribosome. Nat Struct Mol Biol 22, 336-41 (2015).
45. Shoji, S., Dambacher, C. M., Shajani, Z., Williamson, J. R. & Schultz, P. G. Systematic chromosomal deletion of bacterial ribosomal protein genes. J Mol Biol 413, 751-61 (2011).
46. Dabbs, E. R. Selection for Escherichia coli mutants with proteins missing from the ribosome. J Bacteriol 140, 734-7 (1979).
47. Hubbard, B. P. et al. Continuous directed evolution of DNA-binding proteins to improve TALEN specificity. Nature methods 12, 939 (2015).
48. Carlson, J. C., Badran, A. H., Guggiana-Nilo, D. A. & Liu, D. R. Negative selection and stringency modulation in phage-assisted continuous evolution. Nat Chem Biol 10, 216-22 (2014).
49. Asai, T. et al. Construction and initial characterization of Escherichia coli strains with few or no intact chromosomal rRNA operons. Journal of bacteriology 181, 3803-3809 (1999).
50. Asai, T., Zaporojets, D., Squires, C. & Squires, C. L. An Escherichia coli strain with all chromosomal rRNA operons inactivated: complete exchange of rRNA genes between bacteria. Proceedings of the National Academy of Sciences 96, 1971-1976 (1999).
51. Sprouffske, K. Using Growthcurver. (Jul. 30, 2018).
52. Parks, D. H. et al. A standardized bacterial taxonomy based on genome phylogeny substantially revises the tree of life. Nature Biotechnology 36, 996-1004 (2018).
53. Paradis, E. & Schliep, K. ape 5.0: an environment for modern phylogenetics and evolutionary analyses in R. Bioinformatics 35, 526-528 (2019).
54. Letunic, I. & Bork, P. Interactive Tree Of Life (iTOL) v4: recent updates and new developments. Nucleic Acids Res 47, W256-w259 (2019).
55. Subach, O. M., Cranfill, P. J., Davidson, M. W. & Verkhusha, V. V. An enhanced monomeric blue fluorescent protein with the high chemical stability of the chromophore. PLoS One 6, e28674, (2011).
56. Nagai, T. et al. A variant of yellow fluorescent protein with fast and efficient maturation for cell-biological applications. Nat Biotechnol 20, 87-90, (2002).
57. Shaner, N. C. et al. Improved monomeric red, orange and yellow fluorescent proteins derived from Discosoma sp. red fluorescent protein. Nature Biotechnology 22, 1567-1572, (2004).
58. Tomosugi, W. et al. An ultramarine fluorescent protein with increased photostability and pH insensitivity. Nature Methods 6, 351-353, (2009).
59. Rizzo, M. A. & Piston, D. W. High-contrast imaging of fluorescent protein FRET by fluorescence polarization microscopy. Biophys J 88, L14-16, (2005).
60. Karasawa, S., Araki, T., Nagai, T., Mizuno, H. & Miyawaki, A. Cyan-emitting and orange-emitting fluorescent proteins as a donor/acceptor pair for fluorescence resonance energy transfer. Biochem J 381, 307-312, (2004).
61. Cubitt, A. B., Woollenweber, L. A. & Heim, R. Understanding structure-function relationships in the Aequorea victoria green fluorescent protein. Methods Cell Biol 58, 19-30 (1999).
62. Hoi, H. et al. An engineered monomeric Zoanthus sp. yellow fluorescent protein. Chem Biol 20, 1296-1304, (2013).
63. Bindels, D. S. et al. mScarlet: a bright monomeric red fluorescent protein for cellular imaging. Nature Methods 14, 53-56, (2016).
64. Piatkevich, K. D. et al. Monomeric red fluorescent proteins with a large Stokes shift. Proceedings of the National Academy of Sciences, 107(12), 5369-74, (2010).
65. Shcherbo, D. et al. Near-infrared fluorescent proteins. Nat Methods 7, 827-829, (2010).
66. Strack, R. L. et al. A rapidly maturing far-red derivative of DsRed-Express2 for whole-cell labeling. Biochemistry 48, 8279-8281, (2009).
67. Bajar, B. T. et al. Fluorescent indicators for simultaneous reporting of all four cell cycle phases. Nat Methods 13, 993-996, (2016).
68 Fabritius, A. et al. Imaging-Based Screening Platform Assists Protein Engineering. Cell Chem Biol 25, 15541561.e1558, (2018).
69. Lutz, R. & Bujard, H. Independent and tight regulation of transcriptional units in Escherichia coli via the LacR/O, the TetR/O and AraC/I1-I2 regulatory elements. Nucleic Acids Res 25, 1203-1210, (1997).
70. Song, W. et al. Divergent rRNAs as regulators of gene expression at the ribosome level. Nat Microbiol 4, 515-526 (2019).
71. Hui, A. & de Boer, H. A. Specialized ribosome system: preferential translation of a single mRNA species by a subpopulation of mutated ribosomes in Escherichia coli. Proc Natl Acad Sci USA 84, 4762-6 (1987).
72. Brink, M. F., Verbeet, M. P. & de Boer, H. A. Specialized ribosomes: highly specific translation in vivo of a single targeted mRNA species. Gene 156, 215-22 (1995).
73. Hui, A., Jhurani, P. & de Boer, H. A. Directing ribosomes to a single mRNA species: a method to study ribosomal RNA mutations and their effects on translation of a single messenger in Escherichia coli. Methods Enzymol 153, 432-52 (1987).
74. Lee, K., Holland-Staley, C. A. & Cunningham, P. R. Genetic analysis of the Shine-Dalgamo interaction: selection of alternative functional mRNA-rRNA combinations. Rna 2, 1270-85 (1996).
75. Cochella, L. & Green, R. Isolation of antibiotic resistance mutations in the rRNA by using an in vitro selection system. Proc Natl Acad Sci USA 101, 3786-91 (2004).
76. Wang, H. H. & Church, G. M. Multiplexed genome engineering and genotyping methods applications for synthetic biology and metabolic engineering. Methods Enzymol 498, 409-26 (2011).
77. Datsenko, K. A. & Wanner, B. L. One-step inactivation of chromosomal genes in Escherichia coli K-12 using PCR products. Proc Natl Acad Sci USA 97, 6640-5 (2000).
78. Gupta, P., Kannan, K., Mankin, A. S. & Vazquez-Laslop, N. Regulation of gene expression by macrolide-induced ribosomal frameshifting. Mol Cell 52, 629-42 (2013).
79. Held, W. A., Ballou, B., Mizushima, S. & Nomura, M. Assembly mapping of 30 S ribosomal proteins from Escherichia coli. Further studies. J Biol Chem 249, 3103-11 (1974).
80. Sengupta, J., Agrawal, R. K. & Frank, J. Visualization of protein S1 within the 30S ribosomal subunit and its interaction with messenger RNA. Proc Natl Acad Sci USA 98, 11991-6 (2001).
81. Marzi, S. et al. Structured mRNAs regulate translation initiation by binding to the platform of the ribosome. Cell 130, 1019-31 (2007).

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the instant description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for increasing the activity and/or improving the maturation of a non-host cell ribosomal RNA (rRNA) in a host cell, wherein the non-host cell rRNA is encoded by a nucleic acid sequence comprising both rRNA coding sequences and intergenic sequences, the method comprising replacing the intergenic sequences of the nucleic acid sequence comprising both rRNA coding sequences and intergenic sequences with intergenic sequences of the host cell, thereby increasing the activity and/or improving the maturation of the non-host cell rRNA in the host cell.

2. The method of claim 1, wherein the host cell is Escherichia coli, optionally an E. coli strain comprising a genomic deletion for rRNA sequences, optionally further comprising a counter-selectable plasmid comprising E. coli rRNA sequences, optionally wherein the E. coli strain is SQ171.

3. The method of claim 2, wherein the non-host cell is selected from the group consisting of Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica and Shigella spp. (e.g., Shigella flexneri, Shigella dysenteriae, Shigella sonnei, Shigella boydii).

4. The method of claim 2, wherein the non-host cell is a commensal microbe, optionally wherein the commensal microbe is of a phylum or phyla selected from the group consisting of Firmicutes, Bacteroidetes, Bifidobacteria, Eubacteria, Ruminococcus, Lactobacillus, Peptococcus, Proteobacteria, Verrumicrobia, Actinobacteria, Fusobacteria, and Cyanobacteria, and a combination of phyla thereof.

5. The method of claim 1, wherein the host cell is Bacillus subtilis, optionally a B. subtilis strain comprising a genomic deletion for rRNA sequences, optionally further comprising a counter-selectable plasmid comprising B. subtilis rRNA sequences.

6. The method of claim 5, wherein the non-host cell is selected from the group consisting of Mycobacterium tuberculosis, Bifidobacterium longum, Veillonella parvula, Clostridium difficile, Escherichia coli, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica and Shigella spp. (e.g., Shigella flexneri, Shigella dysenteriae, Shigella sonnei, Shigella boydii).

7. The method of claim 5, wherein the non-host cell is a commensal microbe, optionally wherein the commensal microbe is of a phylum or phyla selected from the group consisting of Firmicutes, Bacteroidetes, Bifidobacteria, Eubacteria, Ruminococcus, Lactobacillus, Peptococcus, Proteobacteria, Verrumicrobia, Actinobacteria, Fusobacteria, and Cyanobacteria, and a combination of phyla thereof.

8. The method of claim 2, wherein the non-host cell is selected from the group consisting of Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris and Haemophilus influenza.

9. The method of claim 1, wherein the nucleic acid sequence comprising both rRNA coding sequences and intergenic sequences comprises non-host cell 165, 23S and 5S rRNA sequences, optionally wherein the non-host cell 165, 23S and 5S rRNA sequences are under the control of an inducible promoter, optionally wherein the inducible promoter is an aTc-inducible promoter or an IPTG-inducible promoter.

10. The method of claim 9, wherein the host cell comprises a nucleic acid sequence comprising an orthogonal-ribosome binding site (o-RBS) positioned upstream of a reporter sequence,
optionally wherein the reporter sequence is a gene encoding a fluorescent protein, optionally wherein the fluorescent protein is selected from the group consisting of Sirius fluorescent protein, mTagBFP2, a blue fluorescent protein (BFP), Sapphire fluorescent protein, mCerulean, a yellow fluorescent protein (YFP), LSS-mKate2, MiCy, a green fluorescent protein (GFP) (optionally, a superfolder green fluorescent protein (sfGFP)), mEmerald, Venus, mPapaya, mScarlet-1, mCherry, mRFP, Katushka-9-5, mCarmine, mMaroon1, and E2-Crimson, or optionally wherein the reporter sequence is a gene encoding a chemiluminescent protein, optionally a luciferase protein, and
optionally wherein the nucleic acid sequence comprising an o-RBS positioned upstream of a reporter sequence is under the control of an inducible promoter, optionally wherein the inducible promoter is an IPTG-inducible promoter or an aTc-inducible promoter, optionally wherein the aTc-inducible promoter is a PLtetO-1 or a PtetA promoter.

11. The method of claim 10, wherein the nucleic acid sequence comprising both rRNA coding sequences and intergenic sequences comprises a non-host cell 16S rRNA sequence further comprising an o-antiRBS sequence.

12. The method of claim 1, wherein non-host cell rRNA activity is increased to 50% or more of the level of an appropriate host cell rRNA control.

13. The method of claim 1, wherein growth of the host cell is improved.

14. A composition selected from the group consisting of:
A nucleic acid sequence comprising an aTc-inducible promoter and 16S, 23S and 5S rRNA coding sequences, wherein the 16S sequence further comprises an o-antiRBS sequence;
A rRNA reporter system comprising:
(a) a first nucleic acid sequence comprising an aTc-inducible promoter and 16S, 23S and 5S rRNA coding sequences, wherein the 16S sequence further comprises an o-antiRBS sequence; and
(b) a second nucleic acid sequence comprising an o-RBS sequence and a reporter sequence;
A host cell comprising a nucleic acid sequence comprising non-host cell 16S, 23S and 5S rRNA coding sequences, wherein the non-host cell is selected from the group consisting of Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii and Shigella spp. (e.g., Shigella flexneri, Shigella dysenteriae, Shigella sonnei, Shigella boydii);
A host cell comprising a nucleic acid sequence comprising non-host cell 16S, 23S and 5S rRNA coding sequences, wherein the non-host cell is a commensal microbe, optionally wherein the commensal microbe is of a phylum or phyla selected from the group consisting of Firmicutes, Bacteroidetes, Bifidobacteria, Eubacteria, Ruminococcus, Lactobacillus, Peptococcus, Proteobacteria, Verrumicrobia, Actinobacteria, Fusobacteria, and Cyanobacteria, and a combination of phyla thereof;
An E. coli cell comprising mutated forms of 23S rRNA genes rrlA, rrlB, rrlC, rrlD, rrlE, rrlG and rrlH; and
A nucleic acid sequence comprising a sequence comprising an o-antiRBS sequence operably linked to a sfGFP sequence having a 5′ and a 3′ end, wherein the 3′ end of the sfGFP sequence is attached to the 5′ end of a reporter nucleic acid sequence having a 5′ and a 3′ end.

15. (canceled)

16. The composition of claim 14, wherein:
the second nucleic acid sequence of the rRNA reporter system comprises an inducible promoter, optionally wherein the inducible promoter is an IPTG-inducible promoter;
the reporter sequence of the rRNA reporter system encodes a protein selected from the group consisting of Sirius fluorescent protein, mTagBFP2, a blue fluorescent protein (BFP), Sapphire fluorescent protein, mCerulean, a yellow fluorescent protein (YFP), LSS-mKate2, MiCy, a green fluorescent protein (GFP), mEmerald, Venus, mPapaya, mScarlet-1, mCherry, mRFP, Katushka-9-5, mCarmine, mMaroon1, E2-Crimson, and luciferase protein;
the aTc-inducible promoter of the rRNA reporter system is a PLtetO-1 or a PtetA promoter;
the rRNA reporter system further comprises a third nucleic acid sequence encoding for S20, S16, S1 and/or S15 r-protein(s);
the 16S, 23S and 5S rRNA coding sequences of the rRNA reporter system are non-E. coli sequences, optionally wherein the first nucleic acid sequence further comprises intergenic sequences, optionally wherein the intergenic sequences are E. coli intergenic sequences;
the rRNA reporter system further comprises a third nucleic acid sequence encoding for non-E. coli S20, S16, S1 and/or S15 r-protein(s) of the same organism as the non-E. coli 16S, 23S and 5S rRNA coding sequences;
in the host cell, the nucleic acid sequence comprising non-host cell 16S, 23S and 5S rRNA coding sequences further comprises intergenic sequences, optionally wherein the intergenic sequences are host cell intergenic sequences;
in the host cell, the non-host cell 16S rRNA sequence further comprises an o-antiRBS sequence;
the host cell further comprises a nucleic acid sequence encoding for S20, S16, S1 and/or S15 r-protein(s) of the non-host cell;
the host cell further comprises a nucleic acid sequence comprising an orthogonal-ribosome binding site (o-RBS) positioned upstream of a reporter sequence, optionally wherein the reporter sequence is a gene encoding a fluorescent protein, optionally wherein the fluorescent protein is selected from the group consisting of Sirius fluorescent protein, mTagBFP2, a blue fluorescent protein (BFP), Sapphire fluorescent protein, mCerulean, a yellow fluorescent protein (YFP), LSS-mKate2, MiCy, a green fluorescent protein (GFP) (optionally, a superfolder green fluorescent protein (sfGFP)), mEmerald, Venus, mPapaya, mScarlet-1, mCherry, mRFP, Katushka-9-5, mCarmine, mMaroon1, and E2-Crimson, or optionally wherein the reporter sequence is a gene encoding a chemiluminescent protein, optionally a luciferase protein, optionally wherein the nucleic acid sequence comprising an o-RBS positioned upstream of a reporter sequence is under the control of an inducible promoter, optionally wherein the inducible promoter is an IPTG-inducible promoter or an aTc-inducible promoter, optionally wherein the aTc-inducible promoter is a PLtetO-1 or a PtetA promoter;
the E. coli cell further comprises a sfGFP reporter;
at least one 23S rRNA gene of the E. coli cell is selected from the group consisting of rrlA, rrlB, rrlC, rrlD, rrlE, rrlG and rrlH comprises an A2058U mutation;
the E. coli cell is erythromycin-resistant;
the E. coli cell further comprises an orthogonal large subunit (LSU) ribosome and/or an orthogonal small subunit (SSU) ribosome;
the sfGFP sequence comprises a sequence encoding for SEQ ID NO: 409 (N-MSKGEELFTG-C), optionally wherein the sfGFP sequence comprises SEQ ID NO: 408 (5′-ATGAGCAAAGGTGAAGAACTGTTTACCGGC-3′);
the sfGFP sequence consists of a sequence encoding for SEQ ID NO: 409 (N-MSKGEELFTG-C), optionally wherein the sfGFP sequence consists of SEQ ID NO: 408 (5′-ATGAGCAAAGGTGAAGAACTGTTTACCGGC-3′);
the reporter nucleic acid sequence encodes a fluorescent protein, optionally wherein the fluorescent protein is selected from the group consisting of Sirius fluorescent protein, mTagBFP2, a blue fluorescent protein (BFP), Sapphire fluorescent protein, mCerulean, a yellow fluorescent protein (YFP), LSS-mKate2, MiCy, a green fluorescent protein (GFP) (optionally, a superfolder green fluorescent protein (sfGFP)), mEmerald, Venus, mPapaya, mScarlet-1, mCherry, mRFP, Katushka-9-5, mCarmine, mMaroon1, and E2-Crimson; and/or
the reporter nucleic acid sequence encodes a chemiluminescent protein, optionally a luciferase protein.

17-27. (canceled)

28. A method selected from the group consisting of:
A method for increasing the activity of a non-host cell ribosomal RNA (rRNA) in a host cell, the method comprising introducing a nucleic acid sequence encoding for S20 and/or S16 r-protein(s) of the non-host cell into the host cell, thereby increasing the activity of the non-host cell rRNA in the host cell;
A method for identifying a compound capable of modulating the rRNA activity of a pathogenic microbe in a host cell comprising (i) a rRNA reporter system comprising a first nucleic acid sequence comprising 16S, 23S and 5S rRNA coding sequences, wherein the 16S sequence further comprises an o-antiRBS sequence; and (ii) a second nucleic acid sequence comprising an o-RBS sequence and a reporter sequence, the method comprising:
(a) contacting the host cell with a test compound; and
(b) measuring modulation of the reporter sequence in the presence of the test compound, as compared to an appropriate control,
thereby identifying the test compound as a compound capable of modulating the rRNA activity of a pathogenic microbe in the host cell;
A method for identifying a compound that does not modulate or only weakly modulates (as compared to a pathogenic microbe) the rRNA activity of a commensal microbe in a host cell comprising (i) a rRNA reporter system comprising a first nucleic acid sequence comprising 16S, 23S and 5S rRNA coding sequences, wherein the 16S sequence further comprises an o-antiRBS sequence; and (ii) a second nucleic acid sequence comprising an o-RBS sequence and a reporter sequence, the method comprising:
(a) contacting the host cell with a test compound; and
(b) measuring modulation of the reporter sequence in the presence of the test compound, as compared to an appropriate control,
thereby identifying the test compound as a compound that does not modulate or only weakly modulates (as compared to a pathogenic microbe) the rRNA activity of the commensal microbe in the host cell;
A method for identifying association between an orthogonal SSU and a host cell LSU, the method comprising:
contacting the E. coli cell of claim 58 comprising a host cell LSU with a nucleic acid sequence that encodes for an orthogonal SSU capable of being expressed in the E. coli cell,
contacting the E. coli cell comprising the orthogonal SSU with erythromycin; and
observing the erythromycin sensitivity of the E. coli cell comprising the orthogonal SSU, wherein:
erythromycin sensitivity of the E. coli cell comprising the orthogonal SSU indicates high levels of exchange between the orthogonal SSU and the host cell LSU; and
erythromycin resistance of the E. coli cell comprising the orthogonal SSU indicates low levels of exchange between the orthogonal SSU and the host cell LSU (i.e., the orthogonal SSU preferentially associates with the host cell LSU),
thereby identifying association between the orthogonal SSU and the host cell LSU; and
A method for enhancing translation in a host cell of an orthogonal nucleic acid comprising a reporter sequence, wherein the reporter sequence has a 5′ end and a 3′ end, the method comprising attaching a sfGFP sequence at the 5′ end of the reporter sequence, thereby enhancing translation of the orthogonal nucleic acid sequence in the host cell.

29. The method of claim 28, wherein:
the method further comprises introducing a nucleic acid sequence encoding for S1 and/or S15 r-protein(s) of the non-host cell into the host cell;
the host cell is Escherichia coli, optionally an E. coli strain comprising a genomic deletion for rRNA sequences, optionally further comprising a counter-selectable plasmid comprising E. coli rRNA sequences, optionally wherein the E. coli strain is SQ171;
the non-host cell is selected from the group consisting of Mycobacterium tuberculosis, Bifidobacterium longum, Veillonella parvula, Clostridium difficile, Bacillus subtilis, Staphylococcus aureus, Enterococcus faecium, Enterococcus faecalis, Bacteroides thetaiotaomicron, Helicobacter pylori, Desulfovibrio bastinii, Desulfovibrio vulgaris, Rickettsia parkeri, Rhodopseudomonas palustris, Caulobacter crescentus, Mariprofundus ferrooxydans, Ghiorsea bivora, Neisseria gonorrhoeae, Burkholderia cenocepacia, Bordetella pertussis, Alcaligenes faecalis, Acinetobacter baumannii, Pseudomonas aeruginosa, Marinospirillum minutulum, Alteromonas macleodii, Vibrio cholerae, Providencia stuartii, Proteus mirabilis, Serratia marcescens, Edwardsiella tarda, Enterobacter cloacae, Klebsiella oxytoca, Klebsiella pneumoniae, Klebsiella aerogenes, Citrobacter freundii, Salmonella enterica, Yersinia pestis, Yersinia pseudotuberculosis, Yersinia enterocolitica, Mycobacterium bovis, Mycobacterium avium, Neisseria meningitidis, Listeria monocytogenes, Streptococcus pyogenes, Campylobacter jejuni, Bacteroides fragilis, Proteus vulgaris, Haemophilus influenza and Shigella spp. (e.g. Shigella flexneri, Shigella dysenteriae, Shigella sonnei, Shigella boydii), or the non-host cell is a commensal microbe, optionally wherein the commensal microbe is of a phylum or phyla selected from the group consisting of Firmicutes, Bacteroidetes, Bifidobacteria, Eubacteria, Ruminococcus, Lactobacillus, Peptococcus, Proteobacteria, Verrumicrobia, Actinobacteria, Fusobacteria, and Cyanobacteria, and a combination of phyla thereof;
the host cell is Bacillus subtilis, optionally a B. subtilis strain comprising a genomic deletion for rRNA sequences, optionally further comprising a counter-selectable plasmid comprising B. subtilis rRNA sequences;
the non-host cell is A. baumannii and the nucleic acid sequence encodes for AbS20 and/or AbS16 r-protein(s);
the non-host cell is A. macleodii and the nucleic acid sequence encodes for AmS20 and AmS16 r-proteins, optionally wherein the nucleic acid sequence further encodes for AmS1 and/or AmS15 r-protein(s);
the non-host cell is V. cholerae or M. minitulum and the nucleic acid sequence encodes for S20, S16, S1 and S15 r-proteins of the non-host cell;
the non-host cell is P. aeruginosa and the nucleic acid sequence encodes for PaS16 and PaS20 r-proteins, optionally wherein the nucleic acid sequence further encodes for PaS1 and/or PaS15 r-protein(s);
the non-host cell is selected from the group consisting of A. faecalis, B. cenocepacia, N. gonnorrheae, M. ferrooxydans, and C. crescentus and the nucleic acid sequence encodes for non-host cell S16 and S20 r-proteins;
the nucleic acid sequence encoding for S20 and/or S16 r-proteins of the non-host cell is under the control of a copy-up variant, optionally RepA E93K or E93R;
the host cell further comprises an o-RBS reporter construct, optionally wherein the reporter of the o-RBS reporter construct is under control of a PLtetO-1 or a PtetA promoter
a nucleic acid sequence comprising non-host cell 165, 23S and 5S rRNA sequences expresses the non-host cell rRNA in the host cell, optionally wherein the non-host cell 165, 23S and 5S rRNA sequences are under the control of an inducible promoter, optionally wherein the inducible promoter is an aTc-inducible promoter or an IPTG-inducible promoter, optionally wherein the host cell comprises a nucleic acid sequence comprising an orthogonal-ribosome binding site (o-RBS) positioned upstream of a reporter sequence, optionally wherein the reporter sequence is a gene encoding a fluorescent protein, optionally wherein the fluorescent protein is selected from the group consisting of Sirius fluorescent protein, mTagBFP2, a blue fluorescent protein (BFP), Sapphire fluorescent protein, mCerulean, a yellow fluorescent protein (YFP), LSS-mKate2, MiCy, a green fluorescent protein (GFP) (optionally, a superfolder green fluorescent protein (sfGFP)), mEmerald, Venus, mPapaya, mScarlet-1, mCherry, mRFP, Katushka-9-5, mCarmine, mMaroon1, and E2-Crimson, or optionally wherein the reporter sequence is a gene encoding a chemiluminescent protein, optionally a luciferase protein, and optionally wherein the nucleic acid sequence comprising an o-RBS positioned upstream of a reporter sequence is under the control of an inducible promoter, optionally wherein the inducible promoter is an IPTG-inducible promoter or an aTc-inducible promoter, optionally wherein the aTc-inducible promoter is a PLtetO-1 or a PtetA promoter; or optionally wherein the nucleic acid sequence comprising non-host cell 16S, 23S and 5S rRNA sequences comprises a non-host cell 16S rRNA sequence further comprising an o-antiRBS sequence;
non-host cell rRNA activity is increased to 50% or more of the level of an appropriate host cell rRNA control;
growth of the host cell is improved;
the test compound reduces pathogenic microbe rRNA activity;
test compound, when administered to the pathogenic microbe, reduces growth of the pathogenic microbe;
the test compound is a small molecule;
the host cell further comprises a nucleic acid sequence encoding for S20, S16, S1 and/or S15 r-protein(s) of the pathogenic microbe;
the first nucleic acid sequence comprising 16S, 23S and 5S rRNA coding sequences further comprises intergenic sequences, optionally wherein the intergenic sequences are host cell intergenic sequences;
the test compound selectively modulates the rRNA activity of the pathogenic microbe in the host cell, as compared to modulation of rRNA activity of a commensal microbe in the host cell, optionally wherein ribosomal components of the pathogenic microbe and the commensal microbe are multiplexed within the host cell;
the sfGFP sequence comprises a sequence encoding for SEQ ID NO: 409 (N-MSKGEELFTG-C), optionally wherein the sfGFP sequence comprises SEQ ID NO: 408 (5′-ATGAGCAAAGGTGAAGAACTGTTTACCGGC-3′); and/or
the sfGFP sequence consists of a sequence encoding for SEQ ID NO: 409 (N-MSKGEELFTG-C), optionally wherein the sfGFP sequence consists of SEQ ID NO: 408 (5′-ATGAGCAAAGGTGAAGAACTGTTTACCGGC-3′).

30-71. (canceled)
